(19) United States
(12) Patent Application Publication
(10) Pub. No.: US 2023/0242676 A1
(43) Pub. Date: Aug. 3, 2023

(54) POLYPEPTIDES, PROTEIN COMPLEXES AND METHOD FOR MAKING SAME

(71) Applicant: KISOJI BIOTECHNOLOGY INC., Montréal (CA)

(72) Inventors: Richard WARGACHUCK, Montréal (CA); Ashwani GUPTA, Montréal (CA); Luis DA CRUZ, Montréal (CA); Davis S. YOUNG, Montréal (CA); Shugang YAO, Montréal (CA)

(21) Appl. No.: 17/786,708

(22) PCT Filed: Dec. 18, 2020
(86) PCT No.: PCT/CA2020/051753

§ 371 Date: Dec. 20, 2022

Related U.S. Application Data
(60) Provisional application No. 62/951,701, filed on Dec. 20, 2019.

(51) Int. Cl. C07K 16/46; A61P 35/00; C07K 16/28; C07K 16/40

(52) U.S. Cl. CPC C07K 16/468; A61P 35/00; C07K 16/2827; C07K 16/2809; C07K 16/2818; C07K 16/2896; C07K 16/40; C07K 16/286; A61K 2039/505

(57) ABSTRACT

The present disclosure generally relates to polypeptides that comprise one or more antigen binding domains and a dimerization domain that allow assembly of at least two polypeptide chains into a multivalent and/or multispecific protein complex. The polypeptides and protein complexes of the present disclosure possess anti-tumor activity.

### TECHNICAL FIELD

The present disclosure generally relates to polypeptides that comprise one or more antigen binding domains and a dimerization domain that allow assembly of at least two polypeptide chains into a multivalent and/or multispecific protein complex. The polypeptides and protein complexes of the present disclosure possess anti-tumor activity.

### BACKGROUND

Camelids and cartilaginous fishes naturally produce antibodies composed of functional homodimeric heavy chain antibodies (HCAbs) (Hamers-Casterman et al., 1993; Muyldermans and Smider, 2016). The heavy chains of HCAbs lack the first constant domain (CH1) and differs from classical antibodies by only a few amino acids substitutions normally involved in light chain pairing (Muyldermans et al., 1994; Vu et al., 1997). These substitutions (Val37Phe/Tyr, Gly44Glu, Leu45Arg, and Trp47Gly) are present in framework region 2 (FR2). The antigen-binding fragment of HCAbs is referred to as single domain antibody (sdAb), VHH or Nanobody®. VHHs have a molecular weight of around 15 kDa which makes them amenable to applications that require enhanced tissue penetration or rapid clearance, such as radioisotope-based imaging. However, for therapeutic applications, the VHH half-life usually needs to be increased so as to minimize renal clearance and optimize therapeutic efficacy (De Vlieger et al., Antibodies 8(1), 1-22, 2019). Although methods to increase VHH half-life such as PEGylation, N-glycosylation, HSA or other carrier protein fusions have been exploited, such construct can introduce immunogenicity or have limited success.

VHHs have been exploited as building blocks to make bispecific and multi-specific antibodies. In some studies, bivalent constructs have been shown to be have increased avidity or affinity compared to the monovalent form (Conrath et al., 2001; Coppieters et al., 2006; Hmila et al., 2008; Simmons et al., 2006 and Hultberg et al., 2011, Jähnichen et al. (2010), Fridy et al., 2014).

A number of VHH-based therapeutics are currently in late investigational stage or have been approved by FDA. These include the bivalent monospecific antibody Caplacizumab against antigen vWF approved for Thrombotic thrombocytopenic purpura (Duggan, 2018). A Trivalent nanobody complex, ALX-0171 against RSV is at late-stage development for Respiratory syncytial virus infection (Detallea et al., 2015). ALX-0061 is a monovalent against antigen IL-6R but attached with HSA nanobody to extend half-life and is at clinical development stage for RA and SLE indications (Van Roy et al., 2015). The investigational drug ALX-0761 contains three nanobodies against antigens IL-17A, IL-17F and HAS and is being developed for Psoriasis (Svecova et., 2019). Anti-RANKL, ALX-0141 is a bivalent for antigen RANKL and attached to HSA to extend half-life (Schoen et al., 2013). Ozoralizumab is bivalent nanobody against antigen TNFα and attached to HSA to extend half-life (Fleischmann et al., 2012).

Despite these developments, there remains a need for antibody-like molecules that binds multiple targets and that generate an efficient immune response.

### SUMMARY

The Applicant has generated polypeptides that comprise antigen binding domains and a dimerization domain that allow assembly of two polypeptide chains to form a multivalent and/or multispecific protein complex.

The polypeptides of the present disclosure are composed of different modules and include antigen binding domains that are selected for their ability to bind specific targets. The antigen binding domains may also be selected for their in vivo and/or in vitro functional properties or biological effects including, for example, their ability to modulate cellular processes such as gene expression, signal transduction, cell growth, cell viability and the like.

The antigen binding domains are engineered into a single polypeptide chain so as to target different cellular components or different cell types with a single moiety. The polypeptide chains can be assembled into protein complexes such as dimers to potentiate their biological effect. Several cellular processes can thus be modulated with administration of a single polypeptide, or protein complex species. In addition, the presence of multiple target-specific antigen binding domains within the same molecule ensures that they are delivered, ultimately, to the same location when all cells addressed by each antigen binding domain come together. An additional benefit is that the various biological effects are triggered in a timely fashion or almost concomitantly.

This represents significant advantages over administration of separate antibodies, since several parameters, including dosages, schedule of administration, route of administration, pharmacodynamics, pharmacokinetics which affects the outcome of such administration is accounted for by a single moiety rather than accounting for each moiety separately. As such, administration of separate antibodies does not always achieve the desired biological effect.

Moreover, the polypeptide chains and protein complexes are amenable to conjugation with therapeutics or with detectable moieties.

Another benefit of the polypeptides and protein complexes disclosed herein is that the binding of the various antigen binding domains to their targets may occur in a coordinated fashion. For example, the binding of a given single domain antibody to its target may help the binding of the others.

Based on the present disclosure, the Applicant has generated polypeptides and protein complexes that are composed of various single domain antibodies that target tumors and/or modulate immune checkpoints and/or recruit immune cells. For example, in some embodiments, the polypeptide moieties and protein complexes are composed of various single domain antibodies that target tumors. In some embodiments, the polypeptide moieties and protein complexes are composed of various single domain antibodies that modulate immune checkpoints. In some embodiments, the polypeptide moieties and protein complexes are composed of various single domain antibodies that recruit immune cells. In some embodiments, the polypeptide moieties and protein complexes are composed of various single domain antibodies that target tumors, modulate immune checkpoints and recruit immune cells.

The Applicant demonstrates that the polypeptide chains of the present disclosure promote tumor regression in in vivo preclinical models either alone or in combination with chemotherapy.

Another advantage is that the polypeptide chains of the present disclosure is efficiently expressed in cells. The format of the polypeptide chains disclosed herein allows to achieve yields of protein complexes in the range of gram(s)/L.

The Applicant has also surprisingly discovered a method of producing modular, multifunctional, multispecific and/or multivalent polypeptides that have various advantageous properties over monovalent polypeptides, including for example, increased avidity, and increased specificity of cell targeting.

The VHH, single domain Ab binding moiety incorporated into the polypeptides of the present disclosure do not require light chain for antigen binding, which reduces molecular weight, size, complexity, and number of disulfide bonds compared to binding moieties which require light chain. This, in turn, has various advantages, for example, simplifying manufacturing of quantities suitable for anti-cancer therapy. The CH2-CH3 domain incorporated into the polypeptide of the present disclosure is useful for standard antibody purification processes and confers the half-life of full-size antibodies which are longer than those of VHH proteins. The different size of each chain of the polypeptides of the present disclosure simplifies the distinction between heterodimers and homodimers and contributes to simplifying manufacturing of these antibodies. Another desirable property is the linker used at different positions of the polypeptide of the present disclosure. By selecting linkers not specifically designed to be susceptible to cleavage by proteases this disclosure overcomes the disruption of a multi-specific antibody into its component parts which would otherwise defeat the benefits of being multi-specific.

Another advantage of the polypeptides of the present disclosure is the structure of the polypeptides. This advantage includes, for example, overcoming limitations of lower binding affinities when binding moieties are located on C-terminal end of a polypeptide through linker and antibody optimization. Additional advantages, including for example, that the CH2-CH3 domains engage with various receptors of the immune system and impose spatial organization of binding moieties. Spatial organization overcomes the limitation of linearly arranging binding moieties end-to-end where control of the behaviour of molecules in the middle becomes more difficult with increased number of binding moieties.

The functional property of the single domain antibodies is retained within the polypeptide chain and even upon assembly of polypeptide chains into dimeric protein complexes. Moreover, the functional property of single domain antibodies is retained even when located at the C-terminus of the dimerization domain (e.g., Fc) or between other modules as described herein. Accordingly, in some embodiments, the single domain antibodies described herein retain function when located at the C-terminus of the dimerization domain. In some embodiments, the single domain antibodies described herein retain function when located between modules.

The dimerization domain is based on the CH2-CH3 domains of a natural antibody or contains unique sets of CH3 mutations that favor heterodimer formations.

The protein complex thus generated can comprise at least three, four, five, six and more antigen binding domains each having a desired specificity. In some embodiments, each antigen binding domain of the polypeptide or protein complex is capable of binding its target as a single chain.

Moreover, the Applicant was able to identify linkers and polypeptide configuration that positively influence the activity of the polypeptide or protein complex.

The Applicant has also provided a modular system for making polypeptides of the present disclosure.

The modular system disclosed herein is composed of various DNA segments each comprising a unique overhang that allows assembly at a unique position into a DNA construct encoding the polypeptide. The unique feature of this system allows users to exchange one or more modules to select the best candidate.

In some aspects and embodiments, the disclosure therefore relates to a polypeptide that comprises in a N- to C-terminal fashion an amino acid sequence having the configuration set forth in formula Ia:

X-[(Abₐ)-(L_{b})]ₘ-((DD)-[(L_{c})-(Ab_{d})]ₙ-Y

Wherein m may be 0, 1 or an integer greater than 1;

Wherein n may be 0, 1 or an integer greater than 1, provided that m and n are not 0 simultaneously;

Wherein Abₐ, Ab_{d}, each may independently comprise an antigen binding domain comprising one or more complementarity determining regions (CDRs) of an antibody;

Wherein X or Y may independently be present or absent and may comprise an amino acid sequence;

Wherein L_{b}, L_{c}, may each independently comprise one or more linkers; and

Wherein DD comprises a dimerization domain.

In other aspects and embodiments, the disclosure relates to a polypeptide comprising in a N- to C-terminal fashion an amino acid sequence having the configuration set forth in formula Ib:

X-[(Abₐ)-(L_{b})]ₘ-(DD)-[(L_{c})-(Ab_{d})]ₙ-Y

Wherein m is 0, 1 or an integer greater than 1;

Wherein n is 2 or an integer greater than 2;

Wherein Abₐ, Ab_{d}, each independently comprise an antigen binding domain comprising one or more complementarity determining regions (CDRs) of an antibody;

Wherein X or Y are independently present or absent and comprises an amino acid sequence;

Wherein L_{b}, L_{c}, each independently comprises one or more linkers;

Wherein L_{c} does not comprise a cleavable linker; and

Wherein DD comprises a dimerization domain.

In some embodiments, the dimerization domain of the polypeptide comprises a CH2 domain, a CH3 domain or a combination thereof.

In some embodiments, the dimerization domain comprises a natural IgG1 CH3 domain.

In other embodiments, the dimerization domain comprises a natural IgG4 CH3 domain.

In other embodiments, the dimerization domain comprises a CH3 domain comprising one or more mutations in comparison with the CH3 domain of a natural IgG.

In some embodiments, the dimerization domain is a mutated IgG1 CH3 domain.

In other embodiments, the dimerization domain is a mutated IgG4 CH3 domain.

In some embodiments, the dimerization domain is a first dimerization domain (DD₁) as defined herein. Accordingly, in some embodiments, the dimerization domain is a first dimerization domain (DD₁) having the amino acid sequence disclosed herein.

In some embodiments, the dimerization domain is a second dimerization domain (DD₂) as defined herein. Accordingly, in some embodiments, the dimerization domain is a second dimerization domain (DD₂) having the amino acid sequence disclosed herein.

In some embodiments, the dimerization domain comprises a CH3 domain comprising one or more mutations at positions corresponding to 399, 356 and/or 370 in accordance with EU numbering. In other embodiments, the dimerization domain comprises a CH3 domain comprising one or more mutations at positions corresponding to 399, 357 and/or 439 in accordance with EU numbering.

Accordingly, in some embodiments, the dimerization domain comprises a CH3 domain comprising one or more mutations at positions corresponding to D399, D/E356 and/or K370 in accordance with EU numbering. In yet other embodiments, the dimerization domain comprises a CH3 domain comprising or one or more mutations at positions corresponding to D399, E357 and/or K439 in accordance with EU numbering.

In some embodiments, the CH3 domain may comprise an amino acid substitution at position 356.

In some embodiments, the CH3 domain may comprise an amino acid substitution at position 357.

In some embodiments, the CH3 domain may comprise an amino acid substitution at position 370.

In some embodiments, the CH3 domain may comprise an amino acid substitution at position 399.

In some embodiments, the CH3 domain may comprise an amino acid substitution at position 439.

In some embodiments, the dimerization domain comprises a mutated CH3 domain having amino acid substitutions at positions 399 and 356. In some embodiments, the dimerization domain comprises a mutated CH3 domain having amino acid substitutions at positions 399 and 357. In some embodiments, the dimerization domain comprises a mutated CH3 domain having amino acid substitutions at positions 399 and 370. In some embodiments, the dimerization domain comprises a mutated CH3 domain having amino acid substitutions at positions 399 and 439. In some embodiments, the dimerization domain comprises a mutated CH3 domain having amino acid substitutions at positions 356 and 370. In some embodiments, the dimerization domain comprises a mutated CH3 domain having amino acid substitutions at positions 357 and 439.

In some embodiments, the dimerization domain comprises a mutated CH3 domain having amino acid substitutions at position D399, D/E356 and/or K370 or D399, E357 and/or K439 and one or more further amino acid substitutions.

In some embodiments, the one or more further amino acid substitutions in the mutated CH3 domain may be located in the region encompassing amino acid residues 349 to 355 in accordance with EU numbering.

In some embodiments, the one or more further amino acid substitutions in the mutated CH3 domain may be located in the region encompassing amino acid residues 394 to 395 in accordance with EU numbering.

In some embodiments, the one or more further amino acid substitutions in the mutated CH3 domain may be located in the region encompassing amino acids 349 to 355 and/or in the region encompassing amino acids 394 to 395 in accordance with EU numbering.

In some embodiments, the one or more further amino acid substitutions in the mutated CH3 domain may be at positions corresponding to 349, 350, 351, 352, 354, 355, 394 and/or 395 in accordance with EU numbering.

In some embodiments, the one or more further amino acid substitutions in the mutated CH3 domain may be at positions corresponding to Y349, T350, L351, P352, 5354, R355 or Q355, T394 or P395 in accordance with EU numbering.

In some embodiments, the further amino acid substitution is at position Y349.

In some embodiments, the further amino acid substitution is at position T350.

In some embodiments, the further amino acid substitution is at position L351.

In some embodiments, the further amino acid substitution is at position P352.

In some embodiments, the further amino acid substitution is at position S354.

In some embodiments, the further amino acid substitution is at position R355.

In some embodiments, the further amino acid substitution is at position Q355.

In some embodiments, the further amino acid substitution is at position T394.

In some embodiments, the further amino acid substitution is at position P395.

In some embodiments, the further amino acid substitutions are at positions Y349 and 5354.

In some embodiments, the CH3 domain may comprise amino acid substitutions at positions D399, D/E356, K370 and Y349.

In some embodiments, the CH3 domain may comprise amino acid substitutions at positions D399, D/E356, K370 and T350.

In some embodiments, the CH3 domain may comprise amino acid substitutions at positions D399, D/E356, K370 and L351.

In some embodiments, the CH3 domain may comprise amino acid substitutions at positions D399, D/E356, K370 and P352.

In some embodiments, the CH3 domain may comprise amino acid substitutions at positions D399, D/E356, K370 and S354.

In some embodiments, the CH3 domain may comprise amino acid substitutions at positions D399, D/E356, K370 and R355.

In some embodiments, the CH3 domain may comprise amino acid substitutions at positions D399, D/E356, K370 and Q355.

In some embodiments, the CH3 domain may comprise amino acid substitutions at positions D399, D/E356, K370 and T394.

In some embodiments, the CH3 domain may comprise amino acid substitutions at positions D399, D/E356, K370, Y349 and S354.

In some embodiments, the CH3 domain may comprise amino acid substitutions at positions D399, K439, E357 and Y349.

In some embodiments, the CH3 domain comprises amino acid substitutions at positions D399, E357, K439 and T350.

In some embodiments, the CH3 domain comprises amino acid substitutions at positions D399, E357, K439E and L351.

In some embodiments, the CH3 domain may comprise amino acid substitutions at positions D399, K439, E357 and P352.

In some embodiments, the CH3 domain may comprise amino acid substitutions at positions D399, K439, E357 and 5354.

In some embodiments, the CH3 domain may comprise amino acid substitutions at positions D399, K439, E357 and R355.

In some embodiments, the CH3 domain may comprise mutations at positions D399, K439, E357 and Q355.

In some embodiments, the CH3 domain may comprise amino acid substitutions at positions D399, K439, E357 and P395.

In some embodiments, the CH3 domain comprises amino acid substitutions at positions D399, E357, K439, Y349 and S354.

In some embodiments, the amino acid substitution at position Y349 is selected from Y349K, Y349D or Y349R. More particularly, in some embodiments, the amino acid substitution at position Y349 is Y349K. In other embodiments, the amino acid substitution at position Y349 is Y349D.

In some embodiments, the amino acid substitution at position S354 is selected from S354K, S354D, S354W or S354M. More particularly, in some embodiments, the amino acid substitution at position S354 is S354K. In other embodiments, the amino acid substitution at position S354 is S354D. In other embodiments, the amino acid substitution at position S354 is S354M.

In some embodiments, the amino acid substitution at position L351 is L351Y, L351W, L351H, L351R, L351D, L351A, L351T. More particularly, in some embodiments, the amino acid substitution at position L351 is L351Y. In other embodiments, the amino acid substitution at position L351 is L351W. In other embodiments, the amino acid substitution at position L351 is L351R.

In some embodiments, the amino acid substitution at position T350 is T350L, T350I or T350V. More particularly, in some embodiments, the amino acid substitution at position T350 is T350I. In other embodiments, the amino acid substitution at position T350 is T350V.

In some embodiments, the amino acid substitution at position P352 is P352Y, P352V, P352R, P352T, P352L, P352G, P352E, P352C, P352K or P352D. More particularly, in some embodiments, the amino acid substitution at position P352 is P352R. In other embodiments, the amino acid substitution at position P352 is P352E.

In some embodiments, the amino acid substitution at position T394 is T394N.

In some embodiments, the amino acid substitution at position P395 is P395I. In other embodiments, the amino acid substitution at position P395 is P395G. In other embodiments, the amino acid substitution at position P395 is P395E.

In some embodiments, the amino acid substitution at position R355 is R355K. In other embodiments, the amino acid substitution at position R355 is R355W.

In some embodiments, the amino acid substitution at position Q355 is Q355K. In other embodiments, the amino acid substitution at position Q355 is Q355W.

In yet other aspects and embodiments, the disclosure relates to a polypeptide comprising in a N- to C-terminal fashion an amino acid sequence having the configuration set forth in formula Ic:

X-[(Abₐ)-(L_{b})]ₘ-(DD)-[(L_{c})-(Ab_{d})]ₙ-Y

Wherein m is 0, 1 or an integer greater than 1;

Wherein n is 0, 1 or an integer greater than 1, provided that m and n are not 0 simultaneously;

Wherein Abₐ, Ab_{d}, each independently comprise an antigen binding domain comprising one or more complementarity determining regions (CDRs) of an antibody;

Wherein X or Y are independently present or absent and comprises an amino acid sequence;

Wherein L_{b}, L_{c}, each independently comprises one or more linkers;

Wherein DD comprises a dimerization domain comprising: a) a CH3 domain comprising one or more mutations at positions corresponding to D399, D/E356 and/or K370 in accordance with EU numbering; or b) a CH3 domain comprising one or more mutations at positions corresponding to D399, E357 and/or K439 in accordance with EU numbering.

In some embodiment, the CH3 domain comprises mutations D399N, E356Q and K370E in accordance with EU numbering.

In other embodiments, the CH3 domain comprises mutations D399N, E357Q and K439E in accordance with EU numbering.

In some embodiments, the CH3 domain may comprise mutations D399Q, D/E356Q, K370E, Y349K and S354K.

In some embodiments, the CH3 domain may comprise mutations D399N, D/E356Q, K370E and L351W.

In some embodiments, the CH3 domain may comprise mutations D399N, D/E356Q, K370E and S354M.

In some embodiments, the CH3 domain may comprise mutations D399N, D/E356Q, K370E and T350I.

In some embodiments, the CH3 domain may comprise mutations D399N, D/E356Q, K370E and T350V.

In some embodiments, the CH3 domain may comprise mutations D399N, D/E356Q, K370E and P352R.

In some embodiments, the CH3 domain may comprise mutations D399N, D/E356Q, K370E and P352E.

In some embodiments, the CH3 domain may comprise mutations D399Q, D/E356Q and K370E.

In some embodiments, the CH3 domain may comprise mutations D399N, D/E356Q, K370E and L351Y.

In some embodiments, the CH3 domain may comprise mutations D399N, D/E356Q, K370E, and L351H.

In some embodiments, the CH3 domain may comprise mutations D399N, D/E356Q, K370E, and R355K.

In some embodiments, the CH3 domain may comprise mutations D399N, D/E356Q, K370E, and Q355K.

In some embodiments, the CH3 domain may comprise mutations D399N, D/E356Q, K370E and S354K.

In some embodiments, the CH3 domain may comprise mutations D399N, D/E356Q, K370E and T350L.

In some embodiments, the CH3 domain may comprise mutations D399N, D/E356Q, K370E and T394N.

In some embodiments, the CH3 domain may comprise mutations D399N, D/E356Q, K370E and P352Y.

In some embodiments, the CH3 domain may comprise mutations D399N, D/E356Q, K370E and P352V.

In some embodiments, the CH3 domain may comprise mutations D399N, D/E356Q, K370E and P352T.

In some embodiments, the CH3 domain may comprise mutations D399N, D/E356Q, K370E and P352L.

In some embodiments, the CH3 domain may comprise mutations D399N, D/E356Q, K370E and P352G.

In some embodiments, the CH3 domain may comprise mutations D399N, D/E356Q, K370E and P352C.

In some embodiments, the CH3 domain may comprise mutations D399N, D/E356Q, K370E and L351T.

In some embodiments, the CH3 domain may comprise mutations D399N, D/E356Q, K370E and L351A.

In some embodiments, the CH3 domain comprises mutations D399Q, E357Q, K439E, Y349D and S354D.

In some embodiments, the CH3 domain comprises mutations D399N, E357Q, K439E and L351R.

In some embodiments, the CH3 domain comprises mutations D399N, E357Q, K439E and L351Y.

In some embodiments, the CH3 domain comprises mutations D399N, E357Q, K439E and T350I.

In some embodiments, the CH3 domain may comprise mutations D399N, E357Q, K439E and T350V.

In some embodiments, the CH3 domain may comprise mutations D399Q, K439E, E357Q.

In some embodiments, the CH3 domain may comprise mutations D399N, K439E, E357Q, S354K.

In some embodiments, the CH3 domain may comprise mutations D399N, K439E, E357Q, S354W.

In some embodiments, the CH3 domain may comprise mutations D399N, K439E, E357Q, Y349R.

In some embodiments, the CH3 domain may comprise mutations D399N, K439E, E357Q, T350L.

In some embodiments, the CH3 domain may comprise mutations D399N, K439E, E357Q, R355W.

In some embodiments, the CH3 domain may comprise mutations D399N, K439E, E357Q, Q355W.

In some embodiments, the CH3 domain may comprise mutations D399N, K439E, E357Q, P395I.

In some embodiments, the CH3 domain may comprise mutations D399N, K439E, E357Q, P395G.

In some embodiments, the CH3 domain may comprise mutations D399N, K439E, E357Q, P395E.

In some embodiments, the CH3 domain may comprise mutations D399N, K439E, E357Q, P352K.

In some embodiments, the CH3 domain may comprise mutations D399N, K439E, E357Q, P352D.

In some embodiments, the CH3 domain may comprise mutations D399N, K439E, E357Q, L351D.

In some embodiments, the polypeptide comprises at least two or at least three antigen binding domains and a dimerization domain that allow assembly of two polypeptide chains to form a multivalent and/or multispecific protein complex.

In some embodiments, Lc comprises a non-cleavable linker. In other embodiments, Lc consists of a non-cleavable linker.

In some embodiments where m is 2 or an integer greater than 2, the [(Abₐ)-(L_{b})] units is the same.

In other embodiments where m is 2 or an integer greater than 2, the [(Abₐ)-(L_{b})] units of the polypeptide or protein complex is different.

In other embodiments where m is an integer greater than 2, the [(Abₐ)-(L_{b})] units of the polypeptide or protein complex may comprise the same and different units.

In some embodiments where n is 2 or an integer greater than 2, the [(L_{c})-(Ab_{d})] units are the same.

In other embodiments where n is 2 or an integer greater than 2, the [(L_{c})-(Ab_{d})] units are different.

In other embodiments where n is 2 or an integer greater than 2, the [(L_{c})-(Ab_{d})] units comprise the same and different units.

In embodiments, the one or more linkers comprises a hinge region of an antibody or antigen binding fragment thereof.

In some embodiments, the hinge region is from IgG1.

In other embodiments, the hinge region is from IgG2.

In yet other embodiments, the hinge region is from IgG4.

In some embodiments each of the one or more linkers independently has at least 5, 10, 15, 20, 25, 30, 35, 40, 45 or 50 amino acid residues in length.

In some embodiments, each of the one or more linkers is independently a flexible linker, a helical linker, or a rigid linker.

In some embodiments, the linker L_{c} is a rigid linker.

In some embodiments the one or more linkers comprise a flexible linker and/or a rigid linker.

In some embodiments, the flexible linker is a GS linker.

In some embodiments, the flexible linker comprises one or more units of GGGGS.

In some embodiments, the flexible linker comprises at least 2, 3, 4, 5, or more units of GGGGS.

In some embodiments the rigid linker comprises multiple PA repeats.

In some embodiments, the rigid linker is selected from PAPAPKA (SEQ ID NO:8); APAPAPAPAPKA (SEQ ID NO:9); APAPAPAPAPAPAPAPAPAPKA (SEQ ID NO:10); or combinations thereof.

In some embodiments, the helical linker comprises one or more units of EAAAK.

In some embodiments, the helical linker is selected from AEAAAKEAAAKA (SEQ ID NO:12); AEAAAKEAAAKEAAAKA (SEQ ID NO:13); AEAAAKEAAAKEAAAKEAAAKEAAAKA (SEQ ID NO:14); or combinations thereof.

In some embodiments, the dimerization domain comprises an amino acid sequence at least 80%, 85%, 90%, 95%, 96%, 97%, 98%, or 99% identical to SEQ ID NO:27.

In some embodiments the dimerization domain further comprises an amino acid sequence at least 80%, 85%, 90%, 95%, 96%, 97%, 98%, or 99% identical to SEQ ID NO:29.

In some embodiments m is 2.

In other embodiments m is 3.

In yet other embodiments m is 4.

In further embodiments m is 5 In other embodiments m is an integer greater than 5.

In some embodiments n is 2.

In other embodiments n is 3.

In additional embodiments n is 4.

In further embodiments n is 5.

In other embodiments n is an integer greater than 5.

In some embodiments the polypeptide comprises formula II:

X-(Abₐ₁)-(L_{b1})-(DD)-(L_{c1})-(Ab_{d1})-Y
(formula II).

In some embodiments the polypeptide comprises formula III:

X-(Abₐ₁)-(L_{b1})-(DD)-(L_{c1})-(Ab_{d1})-
(L_{c2})-(Ab_{d2})-Y
(formula III).

In some embodiments the polypeptide comprises formula IV:

X-(Abₐ₁)-(L_{b2})-(Abₐ₂)-(L_{b1})-(DD)-
(L_{c1})-(Ab_{d1})-Y
(formula IV).

In some embodiments the polypeptide comprises formula V:

X-(Abₐ₁)-(L_{b2})-(Abₐ₂)-(L_{b1})-(DD)-
(L_{c1})-(Ab_{d1})-(L_{c2})-(Ab_{d2})-Y
(formula V).

In some embodiments the polypeptide comprises formula VI:

X-(Abₐ₁)-(L_{b2})-(Abₐ₂)-(L_{b1})-(DD)-(L_{c1})-
(Ab_{d1})-(L_{c2})-(Ab_{d2})-(L_{c3})-(Ab_{d3})-Y
(formula VI).

In some embodiments the polypeptide comprises formula VII:

X-(Abₐ₁)-(L_{b3})-(Abₐ₂)-(L_{b2})-(Abₐ₃)-(L_{c1})-
(DD)-(L_{c1})-(Ab_{d1})-(L_{c2})-(Ab_{d2})-Y
(formula VII).

In some embodiments the polypeptide comprises formula VIII:

X-(Abₐ₁)-(L_{b3})-(Abₐ₂)-(L_{b2})-(Abₐ₃)-(L_{b1})-(DD)-
(L_{c1})-(Ab_{d1})-(L_{c2})-(Ab_{d2})-(L_{c3})-(Ab_{d3})-Y
(formula VIII).

In some embodiments, L_{c1} is a rigid linker.

In some embodiments, L_{c2} is a rigid linker.

In some embodiments, L_{c3} is a rigid linker.

In some embodiments, L_{c1}, and L_{c2} are rigid linkers.

In some embodiments, L_{c1}, L_{c2} and L_{c3} are rigid linkers.

In embodiments, the antigen binding domain is a single domain antibody (sdAb).

In embodiments, the antigen binding domain is a heavy chain variable region (VH or VHH).

In some embodiment the VHH is derived from humans, from a mouse, from a rat etc.

In some embodiment, the VHH is from a transgenic mouse or rat capable of expressing camelized mouse or rats VHHs, VHHs from other species (e.g., humans etc.) or camelized VHHs from other species (e.g., camelized human VHH etc.).

In embodiments, the antigen binding domain is a light chain variable region (VL or VLL).

In embodiments, the antigen binding domain is a single chain variable fragment (ScFv).

In embodiments, the antigen binding domain is a V_{NAR} fragment.

In other embodiments, the antigen binding domains of the polypeptide comprises a combination of any of single domain antibodies (sdAbs), heavy chain variable regions (VHs or VHHs), light chain variable regions (VLs or VLLs), single chain variable fragments (ScFvs) and/or V_{NAR} fragments.

In some embodiments, the sdAb or VHH is from a Camelidae antibody.

In embodiments, the Camelidae antibody is from a dromedary, a camel, a llama, an alpaca etc.

In other embodiments, the sdAb or VHH is from a cartilaginous fish antibody.

In embodiments, the cartilaginous fish antibody is a shark antibody.

In some embodiments, each individual antigen binding domain specifically binds to a different epitope.

In other embodiments, each individual antigen binding domain specifically binds to a different antigen.

In yet other embodiments, each individual antigen binding domain specifically binds to a different protein.

In some embodiments, the polypeptide comprises at least one antigen binding domain that binds to a protein expressed by a tumor. In other embodiments, the polypeptide comprises at least one antigen binding domain that binds to a protein expressed by a tumor and that modulates its activity.

In some embodiments, the polypeptide comprises at least one antigen binding domain that binds to an immune checkpoint protein. In other embodiments, the polypeptide comprises at least one antigen binding domain that binds to an immune checkpoint protein and that modulates its activity.

In some embodiments, the polypeptide comprises at least one antigen binding domain that binds to an immune cell protein. In other embodiments, the polypeptide comprises at least one antigen binding domain that binds to an immune cell protein and that modulates its activity. Yet in other embodiments, the polypeptide comprises at least one antigen binding domain that binds to or engages and recruits or redirects immune cells.

In some embodiments, the polypeptide comprises at least one antigen binding domain that binds to peripheral blood mononuclear cells (PBMCs). In other embodiments, the polypeptide comprises at least one antigen binding domain that binds to PBMCs and that modulates its activity. Yet in other embodiments, the polypeptide comprises at least one antigen binding domain that binds to PBMCs and that recruits or redirects PBMCs.

In some embodiments, the polypeptide comprises at least one antigen binding domain that binds to a T-cell protein. In other embodiments, the polypeptide comprises at least one antigen binding domain that binds to a T-cell protein and that modulates its activity. Yet in other embodiments, the polypeptide comprises at least one antigen binding domain that binds to a T-cell protein and that recruits or redirects T-cells.

In some embodiments, the polypeptide comprises at least one antigen binding domain that binds to a protein expressed by a tumor and at least one antigen binding domain that binds to and recruits or redirects an immune cell.

In some embodiments, the polypeptide comprises at least one antigen binding domain that binds to a protein expressed by a tumor, at least one antigen binding domain that binds to an immune check point protein and at least one antigen binding domain that binds to and recruits or redirects an immune cell.

In some embodiments, the polypeptide comprises at least one antigen binding domain that modulates immune checkpoint inhibitors.

In some embodiments, the polypeptide comprises at least one antigen binding domain that binds to a tumor antigen, at least one antigen binding domain that binds to an immune check point protein and at least one antigen binding domain that binds to a T-cell.

In some embodiments, the polypeptide comprises at least one antigen binding domain that binds to a tumor antigen, at least one antigen binding domain that binds to an immune check point protein and at least one antigen binding domain that binds to CD3.

In some embodiments, the polypeptide comprises at least one antigen binding domain that modulates CD3 function.

In some embodiments, the polypeptide comprises an antigen binding domain that specifically binds to a receptor.

In embodiments, the receptor is a G-protein coupled receptor.

In embodiments, the G-protein coupled receptor is a dopamine receptor.

In some embodiments, the dopamine receptor is dopamine receptor D1 (DRD1), dopamine receptor D2 (DRD2), dopamine receptor D3 (DRD3), dopamine receptor D4 (DRD4) or dopamine receptor D5 (DRD5).

In some embodiments, the polypeptide comprises an antigen binding domain that specifically binds to a tumor antigen and an antigen binding domain that specifically binds to an immunomodulator.

In some embodiments the antigen binding domain that specifically binds to a tumor antigen is N-terminal to the dimerization domain and the antigen binding domain that specifically binds to an immunomodulator is C-terminal to the dimerization domain.

In some embodiments, the immunomodulator is an immune checkpoint protein, a cytokine, a chemokine or an immune receptor or coreceptor.

In some embodiments, the polypeptide comprises one or more antigen binding domains that specifically bind to CD36, DRD1, DRD2, DRD3, DRD4, DRD5, PD-L1, TROP2, CD147, MCT1, IL1RAP, AMIGO2, PTK7, MCT2, MCT4, NHE1, H+/K+-ATPase, LAP, HLA-I A2, CD73, CD98, CEACAM5/6, ICAM-1, MCSP, fibronectin, Beta 1 Integrin, Tetraspanin 8, CD164, CD59, CD63, CD44, CD166, cWF, TNF, IL-17A, IL17-F, IL-6R, BCMA, TNF, RANKL, ADAMTS5, VEGF, Ang2, CX₃CR1, CXCR4, TfR1 (CD71), CXCR2, CD3, PD1, PDL-1, CTLA-4, CD8, LAG-3, OX40, CD27, CD122/IL2RB, TLR8/CD288, TIM-3, ICOS/CD278, NKG2A, A2AR, B7-H3, GITR/TNFRSF18, 4-IBB/CD137, KIR2DL1, KIR3DL2, SIRPα, CD47, VISTA, CD40, CD112, CD96, TOGOT, BTLA, TIGIT, LAG-3, CD4, VEGFR2, CD19, IGFR1, EpCAM, EGFR, DLL3, CGRP, CD79b, CD28, CCR5, ErbB3, ErbB2, TGFβ1, TGFβ2, TGFβ3, TGFβR1, TGFβR2, IDO1, IDO2, TLR-4, TLR-7, TLR-8, TLR-9, SARS-CoV-1 spike protein, SARS-CoV-2 spike protein or combinations thereof.

In some embodiments, the polypeptide comprises one or more antigen binding domains N-terminal to the dimerization domain that specifically bind to CD36, DRD1, DRD2, DRD3, DRD4, DRD5, PD-L1, TROP2, CD147, MCT1, IL1RAP, AMIGO2, PTK7, MCT2, MCT4, NHE1, H+/K+-ATPase, LAP, HLA-I A2, CD73, CD98, CEACAM5/6, ICAM-1, MCSP, fibronectin, Beta 1 Integrin, Tetraspanin 8, CD164, CD59, CD63, CD44, CD166, cWF, TNF, IL-17A, Th17-F, IL-6R, BCMA, TNF, RANKL, ADAMTS5, VEGF, Ang2, CX₃CR1, CXCR4, TfR1 (CD71), CXCR2, VEGFR2, CD19, IGFR1, EpCAM, EGFR, DLL3, CGRP, CD79b, CD28, CCR5, ErbB3, ErbB2, TGFβ1, TGFβ2, TGFβ3, TGFβR1-TGFβR2, IDOL IDO2, TLR-4, TLR-7, TLR-8, TLR-9 or combinations thereof.

In some embodiments, polypeptide comprises one or more antigen binding domains N-terminal to the dimerization domain that specifically bind to CD36, DRD1, DRD2, PD-L1, or TROP2.

In some embodiments, the polypeptide comprises one or more antigen binding domains C-terminal to the dimerization domain that specifically bind to CD3, PD1, PDL-1, CTLA-4, CD8, LAG-3, OX40, CD27, CD122/IL2RB, TLR8/CD288, TIM-3, ICOS/CD278, NKG2A, A2AR, B7-H3, GITR/TNFRSF18, 4-IBB/CD137, KIR2DL1, KIR3DL2, SIRPα, CD47, VISTA, CD40, CD112, CD96, TOGOT, BTLA, TIGIT, LAG-3, CD4 or combinations thereof.

In some embodiments, the polypeptide comprises one or more antigen binding domains C-terminal to the dimerization domain that specifically bind to CD3, PD1, CTLA-4, CD8, LAG-3, OX40, CD27, CD122/IL2RB, TLR8/CD288, Tim3, ICOS/CD278, NKG2A, A2AR, B7-H3, GITR/TNFRSF18, 4-IBB/CD137, KIR2DL1, KIR3DL2, SIRPα, CD47, VISTA, CD40, CD112, CD96, TOGOT, BTLA or CD4.

It is to be understood herein, that a given antigen binding domain may bind to an epitope that exists in different proteins. As such, in some embodiments, the antigen binding domain, or the polypeptides or protein complexes comprising same, may bind to more than one protein. In some embodiments, the antigen binding domain, or the polypeptides or protein complexes comprising same, may have affinity for more than one protein.

In some embodiments, the polypeptide comprises one or more antigen binding domain that binds a viral antigen. In some embodiments, the viral antigen includes a protein from an enveloped virus. In some embodiments, the viral antigen includes a viral glycoprotein. In some embodiments, the viral antigen includes spike proteins.

In some embodiments, the polypeptide comprises one or more antigen binding domain that binds SARS-CoV proteins. For example, the polypeptide may comprise one or more antigen binding domain that binds to SARS-CoV-1 spike protein. For example, the polypeptide may comprise one or more antigen binding domain that binds to SARS-CoV-2 spike protein.

In some embodiments, the polypeptide comprises at least two antigen binding domains C-terminal to the dimerization domain that specifically bind to CD3 and PD1 respectively.

In some embodiments, one or more of the antigen binding domains is humanized.

In some embodiments, X or Y are independently selected from the group consisting of a linker, a cytokine, a chemokine, a tag, a masking domain, a phage coat protein (pIII, pVI, pV, pVII or pIX), an antigen binding domain or combination thereof.

In some embodiments, the polypeptide is conjugated to a therapeutic moiety, a detectable moiety or to a protein allowing an extended half-life or is attached to nanoparticle.

Other aspects and embodiments of the present disclosure relate to a pharmaceutical composition comprising a polypeptide disclosed herein and a pharmaceutically acceptable carrier.

Yet other aspects and embodiments of the present disclosure relate to a nucleic acid encoding a polypeptide, or polypeptide chain disclosed herein.

In other aspects and embodiments, the present disclosure relates to a nucleic acid encoding individual modules including antigen binding domains, dimerization domains, linkers or combination thereof disclosed herein. The nucleic acid may be in the form of DNA segments as disclosed herein.

Additional aspects and embodiments of the present disclosure relate to a vector comprising a nucleic disclosed herein.

Further aspects and embodiments of the present disclosure relate to a cell expressing the polypeptide disclosed herein.

Additional aspects and embodiments of the present disclosure relate to a cell comprising the nucleic acid or the vector disclosed herein.

Further aspects and embodiments of the present disclosure relate to a kit comprising the polypeptide disclosed herein.

Yet further aspects and embodiments of the present disclosure relate to a kit comprising the nucleic acid, the vector or the cell disclosed herein.

In other aspects and embodiments, the present disclosure relates to a protein complex comprising a first polypeptide chain and a second polypeptide chain disclosed herein. In some embodiments, the first and second polypeptide are identical or different. In some embodiments, the first and second polypeptide comprise identical or different amino acid sequences.

In some embodiments, the protein complexes are made from polypeptide chains that include one antigen binding domain at the N-terminus and one or two antigen binding domains at the C-terminus of the dimerization domain.

The protein complex of the present disclosure may comprise two antigen binding domains that targets different immunomodulators.

In some embodiments, the polypeptide chains include one tumor-targeting antigen binding domain and two antigen binding domains that bind different immunomodulators thereby generating a hexavalent and trispecific protein complex. When two such polypeptide chains comprise a CH3 domain that favorize heterodimer formations a hexavalent and hexaspecific protein complex may be obtained.

In other aspects and embodiments the present disclosure relates to a protein complex comprising a) a first polypeptide comprising one or more antigen binding domains and a first dimerization domain (DD₁) comprising a CH3 domain comprising one or more mutations at positions corresponding to 399, 356 and/or 370 in accordance with EU numbering and b) a second polypeptide comprising one or more antigen binding domains and a second dimerization domain (DD₂) comprising a CH3 domain comprising one or more mutations at positions corresponding to 399, 357 and/or 439 in accordance with EU numbering wherein the first and second polypeptides form a dimer.

In yet other aspects and embodiments the present disclosure relates to a protein complex comprising a) a first polypeptide comprising one or more antigen binding domains and a first dimerization domain (DD₁) comprising a CH3 domain comprising one or more mutations at positions corresponding to D399, D/E356 and/or K370 in accordance with EU numbering and b) a second polypeptide comprising one or more antigen binding domains and a second dimerization domain (DD₂) comprising a CH3 domain comprising one or more mutations at positions corresponding to D399, E357 and/or K439 in accordance with EU numbering wherein the first and second polypeptides form a dimer.

In some embodiments, the first dimerization domain (DD₁) and/or second dimerization domain (DD₂) comprises a CH3 domain comprising further mutations at positions corresponding to 349, 350, 351, 352, 354, 355, 394 and/or 395 in accordance with EU numbering.

In other embodiments, the first dimerization domain (DD₁) and/or second dimerization domain (DD₂) comprises a CH3 domain comprising further mutations at positions corresponding to Y349, T350, L351, P352, S354, R355 or Q355, T394 and/or P395 in accordance with EU numbering.

In some embodiments, the further mutation is in the first dimerization domain at position Y349.

In some embodiments, the further mutation is in the first dimerization domain at position T350.

In some embodiments, the further mutation is in the first dimerization domain at position L351.

In some embodiments, the further mutation is in the first dimerization domain at position P352.

In some embodiments, the further mutation is in the first dimerization domain at position 5354.

In some embodiments, the further mutation is in the first dimerization domain at position R355.

In some embodiments, the further mutation is in the first dimerization domain at position Q355.

In some embodiments, the further mutations are in the first dimerization domain at positions Y349 and S354.

In some embodiments, the further mutation is in the second dimerization domain at position Y349.

In some embodiments, the further mutation is in the second dimerization domain at position T350.

In some embodiments, the further mutation is in the second dimerization domain at position L351.

In some embodiments, the further mutation is in the second dimerization domain at position P352.

In some embodiments, the further mutation is in the second dimerization domain at position S354.

In some embodiments, the further mutation is in the second dimerization domain at position R355.

In some embodiments, the further mutation is in the second dimerization domain at position Q355.

In some embodiments, the further mutation is in the second dimerization domain at position P395.

In some embodiments, the further mutations are in the second dimerization domain at positions Y349 and 5354.

In some embodiments, the first dimerization domain (DD₁) comprises a CH3 domain comprising mutations D399N, D/E356Q and K370E in accordance with EU numbering, and the second dimerization domain (DD₂) comprises a CH3 domain comprising mutations D399N, E357Q and K439E in accordance with EU numbering. Accordingly, in some embodiments, the first dimerization domain (DD₁) comprises a CH3 domain comprising mutations D399N, D/E356Q and K370E in accordance with EU numbering. In some embodiments, the second dimerization domain (DD₂) comprises a CH3 domain comprising mutations D399N, E357Q and K439E in accordance with EU numbering.

In some embodiments, the first dimerization domain (DD₁) comprises a CH3 domain comprising mutations D399Q, D/E356Q, K370E, Y349K and S354K in accordance with EU numbering and the second dimerization domain (DD₂) comprises a CH3 domain comprising mutations D399Q, E357Q, K439E, Y349D and S354D in accordance with EU numbering.

Accordingly, in some embodiments, the first dimerization domain (DD₁) comprises a CH3 domain comprising mutations D399Q, D/E356Q, K370E, Y349K and S354K in accordance with EU numbering. In some embodiments, the second dimerization domain (DD₂) comprises a CH3 domain comprising mutations D399Q, E357Q, K439E, Y349D and S354D in accordance with EU numbering.

In some embodiments, the first dimerization domain (DD₁) comprises a CH3 domain comprising mutations D399N, D/E356Q, K370E and L351W in accordance with EU numbering and the second dimerization domain (DD₂) comprises a CH3 domain comprising mutations D399N, E357Q, K439E and L351R in accordance with EU numbering. Accordingly, in some embodiments, the first dimerization domain (DD₁) comprises a CH3 domain comprising mutations D399N, D/E356Q, K370E and L351W in accordance with EU numbering. In some embodiments, the second dimerization domain (DD₂) comprises a CH3 domain comprising mutations D399N, E357Q, K439E and L351R in accordance with EU numbering.

In some embodiments, the first dimerization domain (DD₁) comprises a CH3 domain comprising mutations D399N, D/E356Q, K370E and S354M in accordance with EU numbering and the second dimerization domain (DD₂) comprises a CH3 domain comprising mutations D399N, E357Q, K439E and L351Y in accordance with EU numbering. Accordingly, in some embodiments, the first dimerization domain (DD₁) comprises a CH3 domain comprising mutations D399N, D/E356Q, K370E and S354M in accordance with EU numbering. In some embodiments, the second dimerization domain (DD₂) comprises a CH3 domain comprising mutations D399N, E357Q, K439E and L351Y in accordance with EU numbering.

In some embodiments, the first dimerization domain (DD₁) comprises a CH3 domain comprising mutations D399N, D/E356Q, K370E and T350I in accordance with EU numbering and the second dimerization domain (DD₂) comprises a CH3 domain comprising mutations D399N, E357Q, K439E and T350I in accordance with EU numbering. Accordingly, in some embodiments, the first dimerization domain (DD₁) comprises a CH3 domain comprising mutations D399N, D/E356Q, K370E and T350I in accordance with EU numbering. In some embodiments, the second dimerization domain (DD₂) comprises a CH3 domain comprising mutations D399N, E357Q, K439E and T350I in accordance with EU numbering.

In some embodiments, the first dimerization domain (DD₁) comprises a CH3 domain comprising mutations D399N, D/E356Q, K370E and T350V in accordance with EU numbering and the second dimerization domain (DD₂) comprises a CH3 domain comprising mutations D399N, E357Q, K439E and T350V in accordance with EU numbering. In some embodiments, the first dimerization domain (DD₁) comprises a CH3 domain comprising mutations D399N, D/E356Q, K370E and T350V in accordance with EU numbering. In some embodiments, the second dimerization domain (DD₂) comprises a CH3 domain comprising mutations D399N, E357Q, K439E and T350V in accordance with EU numbering.

In some embodiments, the first dimerization domain (DD₁) comprises a CH3 domain comprising mutations D399N, D/E356Q, K370E and P352R in accordance with EU numbering and the second dimerization domain (DD₂) comprises a CH3 domain comprising mutations D399N, E357Q, K439E and L351R in accordance with EU numbering. Accordingly, in some embodiments, the first dimerization domain (DD₁) comprises a CH3 domain comprising mutations D399N, D/E356Q, K370E and P352R in accordance with EU numbering. In some embodiments, the second dimerization domain (DD₂) comprises a CH3 domain comprising mutations D399N, E357Q, K439E and L351R in accordance with EU numbering.

In some embodiments, the first dimerization domain (DD₁) comprises a CH3 domain comprising mutations D399N, D/E356Q, K370E and P352E in accordance with EU numbering and the second dimerization domain (DD₂) comprises a CH3 domain comprising mutations D399N, E357Q, K439E and L351R in accordance with EU numbering. Accordingly, in some embodiments, the first dimerization domain (DD₁) comprises a CH3 domain comprising mutations D399N, D/E356Q, K370E and P352E in accordance with EU numbering. In some embodiments, the second dimerization domain (DD₂) comprises a CH3 domain comprising mutations D399N, E357Q, K439E and L351R in accordance with EU numbering.

In some embodiments, the first dimerization domain (DD₁) comprises a CH3 domain comprising mutations D399Q, D/E356Q and K370E in accordance with EU numbering and the second dimerization domain (DD₂) comprises a CH3 domain comprising mutations D399Q, E357Q and K439E in accordance with EU numbering. Accordingly, in some embodiments, the first dimerization domain (DD₁) comprises a CH3 domain comprising mutations D399Q, D/E356Q and K370E in accordance with EU numbering. In some embodiments, the second dimerization domain (DD₂) comprises a CH3 domain comprising mutations D399Q, E357Q and K439E in accordance with EU numbering.

In some embodiments, the first dimerization domain (DD₁) comprises a CH3 domain comprising mutations D399N, D/E356Q, K370E and L351Y in accordance with EU numbering and the second dimerization domain (DD₂) comprises a CH3 domain comprising mutations D399N, E357Q, K439E and S354K in accordance with EU numbering. Accordingly, in some embodiments, the first dimerization domain (DD₁) comprises a CH3 domain comprising mutations D399N, D/E356Q, K370E and L351Y in accordance with EU numbering. In some embodiments, the second dimerization domain (DD₂) comprises a CH3 domain comprising mutations D399N, E357Q, K439E and S354K in accordance with EU numbering.

In some embodiments, the first dimerization domain (DD₁) comprises a CH3 domain comprising mutations D399N, D/E356Q, K370E and L351H in accordance with EU numbering and the second dimerization domain (DD₂) comprises a CH3 domain comprising mutations D399N, E357Q, K439E and S354W in accordance with EU numbering. Accordingly, in some embodiments, the first dimerization domain (DD₁) comprises a CH3 domain comprising mutations D399N, D/E356Q, K370E and L351H in accordance with EU numbering. In some embodiments, the second dimerization domain (DD₂) comprises a CH3 domain comprising mutations D399N, E357Q, K439E and S354W in accordance with EU numbering.

In some embodiments, the first dimerization domain (DD₁) comprises a CH3 domain comprising mutations D399N, D/E356Q, K370E and R355K in accordance with EU numbering and the second dimerization domain (DD₂) comprises a CH3 domain comprising mutations D399N, E357Q, K439E and Y349R in accordance with EU numbering. Accordingly, in some embodiments, the first dimerization domain (DD₁) comprises a CH3 domain comprising mutations D399N, D/E356Q, K370E and R355K in accordance with EU numbering. In some embodiments, the second dimerization domain (DD₂) comprises a CH3 domain comprising mutations D399N, E357Q, K439E and Y349R in accordance with EU numbering.

In some embodiments, the first dimerization domain (DD₁) comprises a CH3 domain comprising mutations D399N, D/E356Q, K370E and Q355K in accordance with EU numbering and the second dimerization domain (DD₂) comprises a CH3 domain comprising mutations D399N, E357Q, K439E and Y349R in accordance with EU numbering. Accordingly, in some embodiments, the first dimerization domain (DD₁) comprises a CH3 domain comprising mutations D399N, D/E356Q, K370E and Q355K in accordance with EU numbering. In some embodiments, the second dimerization domain (DD₂) comprises a CH3 domain comprising mutations D399N, E357Q, K439E and Y349R in accordance with EU numbering.

In some embodiments, the first dimerization domain (DD₁) comprises a CH3 domain comprising mutations D399N, D/E356Q, K370E and S354K in accordance with EU numbering and the second dimerization domain (DD₂) comprises a CH3 domain comprising mutations D399N, E357Q, K439E and T350L in accordance with EU numbering. Accordingly, in some embodiments, the first dimerization domain (DD₁) comprises a CH3 domain comprising mutations D399N, D/E356Q, K370E and S354K in accordance with EU numbering. In some embodiments, the second dimerization domain (DD₂) comprises a CH3 domain comprising mutations D399N, E357Q, K439E and T350L in accordance with EU numbering.

In some embodiments, the first dimerization domain (DD₁) comprises a CH3 domain comprising mutations D399N, D/E356Q, K370E and T350L in accordance with EU numbering and the second dimerization domain (DD₂) comprises a CH3 domain comprising mutations D399N, E357Q, K439E and R355W in accordance with EU numbering. Accordingly, in some embodiments, the first dimerization domain (DD₁) comprises a CH3 domain comprising mutations D399N, D/E356Q, K370E and T350L in accordance with EU numbering. In some embodiments, the second dimerization domain (DD₂) comprises a CH3 domain comprising mutations D399N, E357Q, K439E and R355W in accordance with EU numbering.

In some embodiments, the first dimerization domain (DD₁) comprises a CH3 domain comprising mutations D399N, D/E356Q, K370E and T350L in accordance with EU numbering and the second dimerization domain (DD₂) comprises a CH3 domain comprising mutations D399N, E357Q, K439E and Q355W in accordance with EU numbering. Accordingly, in some embodiments, the first dimerization domain (DD₁) comprises a CH3 domain comprising mutations D399N, D/E356Q, K370E and T350L in accordance with EU numbering. In some embodiments, the second dimerization domain (DD₂) comprises a CH3 domain comprising mutations D399N, E357Q, K439E and Q355W in accordance with EU numbering.

In some embodiments, the first dimerization domain (DD₁) comprises a CH3 domain comprising mutations D399N, D/E356Q, K370E and T394N in accordance with EU numbering and the second dimerization domain (DD₂) comprises a CH3 domain comprising mutations D399N, E357Q, K439E and P395I in accordance with EU numbering. Accordingly, in some embodiments, the first dimerization domain (DD₁) comprises a CH3 domain comprising mutations D399N, D/E356Q, K370E and T394N in accordance with EU numbering. In some embodiments, the second dimerization domain (DD₂) comprises a CH3 domain comprising mutations D399N, E357Q, K439E and P395I in accordance with EU numbering.

In some embodiments, the first dimerization domain (DD₁) comprises a CH3 domain comprising mutations D399N, D/E356Q, K370E and T394N in accordance with EU numbering and the second dimerization domain (DD₂) comprises a CH3 domain comprising mutations D399N, E357Q, K439E and P395G in accordance with EU numbering. Accordingly, in some embodiments, the first dimerization domain (DD₁) comprises a CH3 domain comprising mutations D399N, D/E356Q, K370E and T394N in accordance with EU numbering. In some embodiments, the second dimerization domain (DD₂) comprises a CH3 domain comprising mutations D399N, E357Q, K439E and P395G in accordance with EU numbering.

In some embodiments, the first dimerization domain (DD₁) comprises a CH3 domain comprising mutations D399N, D/E356Q, K370E and T394N in accordance with EU numbering and the second dimerization domain (DD₂) comprises a CH3 domain comprising mutations D399N, E357Q, K439E and P395E in accordance with EU numbering. Accordingly, in some embodiments, the first dimerization domain (DD₁) comprises a CH3 domain comprising mutations D399N, D/E356Q, K370E and T394N in accordance with EU numbering. In some embodiments, the second dimerization domain (DD₂) comprises a CH3 domain comprising mutations D399N, E357Q, K439E and P395E in accordance with EU numbering.

In some embodiments, the first dimerization domain (DD₁) comprises a CH3 domain comprising mutations D399N, D/E356Q, K370E and P352Y in accordance with EU numbering and the second dimerization domain (DD₂) comprises a CH3 domain comprising mutations D399N, E357Q, K439E and P352K in accordance with EU numbering. Accordingly, in some embodiments, the first dimerization domain (DD₁) comprises a CH3 domain comprising mutations D399N, D/E356Q, K370E and P352Y in accordance with EU numbering. In some embodiments, the second dimerization domain (DD₂) comprises a CH3 domain comprising mutations D399N, E357Q, K439E and P352K in accordance with EU numbering.

In some embodiments, the first dimerization domain (DD₁) comprises a CH3 domain comprising mutations D399N, D/E356Q, K370E and P352V in accordance with EU numbering and the second dimerization domain (DD₂) comprises a CH3 domain comprising mutations D399N, E357Q, K439E and P352K in accordance with EU numbering. Accordingly, in some embodiments, the first dimerization domain (DD₁) comprises a CH3 domain comprising mutations D399N, D/E356Q, K370E and P352V in accordance with EU numbering. In some embodiments, the second dimerization domain (DD₂) comprises a CH3 domain comprising mutations D399N, E357Q, K439E and P352K in accordance with EU numbering.

In some embodiments, the first dimerization domain (DD₁) comprises a CH3 domain comprising mutations D399N, D/E356Q, K370E and P352T in accordance with EU numbering and the second dimerization domain (DD₂) comprises a CH3 domain comprising mutations D399N, E357Q, K439E and P352K in accordance with EU numbering. Accordingly, in some embodiments, the first dimerization domain (DD₁) comprises a CH3 domain comprising mutations D399N, D/E356Q, K370E and P352T in accordance with EU numbering. In some embodiments, the second dimerization domain (DD₂) comprises a CH3 domain comprising mutations D399N, E357Q, K439E and P352K in accordance with EU numbering.

In some embodiments, the first dimerization domain (DD₁) comprises a CH3 domain comprising mutations D399N, D/E356Q, K370E and P352R in accordance with EU numbering and the second dimerization domain (DD₂) comprises a CH3 domain comprising mutations D399N, E357Q, K439E and P352K in accordance with EU numbering. Accordingly, in some embodiments, the first dimerization domain (DD₁) comprises a CH3 domain comprising mutations D399N, D/E356Q, K370E and P352R in accordance with EU numbering. In some embodiments, the second dimerization domain (DD₂) comprises a CH3 domain comprising mutations D399N, E357Q, K439E and P352K in accordance with EU numbering.

In some embodiments, the first dimerization domain (DD₁) comprises a CH3 domain comprising mutations D399N, D/E356Q, K370E and P352R in accordance with EU numbering and the second dimerization domain (DD₂) comprises a CH3 domain comprising mutations D399N, E357Q, K439E and P352D in accordance with EU numbering. Accordingly, in some embodiments, the first dimerization domain (DD₁) comprises a CH3 domain comprising mutations D399N, D/E356Q, K370E and P352R in accordance with EU numbering. In some embodiments, the second dimerization domain (DD₂) comprises a CH3 domain comprising mutations D399N, E357Q, K439E and P352D in accordance with EU numbering.

In some embodiments, the first dimerization domain (DD₁) comprises a CH3 domain comprising mutations D399N, D/E356Q, K370E and P352L in accordance with EU numbering and the second dimerization domain (DD₂) comprises a CH3 domain comprising mutations D399N, E357Q, K439E and P352K in accordance with EU numbering. Accordingly, in some embodiments, the first dimerization domain (DD₁) comprises a CH3 domain comprising mutations D399N, D/E356Q, K370E and P352L in accordance with EU numbering. In some embodiments, the second dimerization domain (DD₂) comprises a CH3 domain comprising mutations D399N, E357Q, K439E and P352K in accordance with EU numbering.

In some embodiments, the first dimerization domain (DD₁) comprises a CH3 domain comprising mutations D399N, D/E356Q, K370E and P352G in accordance with EU numbering and the second dimerization domain (DD₂) comprises a CH3 domain comprising mutations D399N, E357Q, K439E and P352K in accordance with EU numbering. Accordingly, in some embodiments, the first dimerization domain (DD₁) comprises a CH3 domain comprising mutations D399N, D/E356Q, K370E and P352G in accordance with EU numbering. In some embodiments, the second dimerization domain (DD₂) comprises a CH3 domain comprising mutations D399N, E357Q, K439E and P352K in accordance with EU numbering.

In some embodiments, the first dimerization domain (DD₁) comprises a CH3 domain comprising mutations D399N, D/E356Q, K370E and P352C in accordance with EU numbering and the second dimerization domain (DD₂) comprises a CH3 domain comprising mutations D399N, E357Q, K439E and P352K in accordance with EU numbering. Accordingly, in some embodiments, the first dimerization domain (DD₁) comprises a CH3 domain comprising mutations D399N, D/E356Q, K370E and P352C in accordance with EU numbering. In some embodiments, the second dimerization domain (DD₂) comprises a CH3 domain comprising mutations D399N, E357Q, K439E and P352K in accordance with EU numbering.

In some embodiments, the first dimerization domain (DD₁) comprises a CH3 domain comprising mutations D399N, D/E356Q, K370E and P352C in accordance with EU numbering and the second dimerization domain (DD₂) comprises a CH3 domain comprising mutations D399N, E357Q, K439E and P352D in accordance with EU numbering. Accordingly, in some embodiments, the first dimerization domain (DD₁) comprises a CH3 domain comprising mutations D399N, D/E356Q, K370E and P352C in accordance with EU numbering. In some embodiments, the second dimerization domain (DD₂) comprises a CH3 domain comprising mutations D399N, E357Q, K439E and P352D in accordance with EU numbering.

In some embodiments, the first dimerization domain (DD₁) comprises a CH3 domain comprising mutations D399N, D/E356Q, K370E and L351T in accordance with EU numbering and the second dimerization domain (DD₂) comprises a CH3 domain comprising mutations D399N, E357Q, K439E and P352K in accordance with EU numbering. Accordingly, in some embodiments, the first dimerization domain (DD₁) comprises a CH3 domain comprising mutations D399N, D/E356Q, K370E and L351T in accordance with EU numbering. In some embodiments, the second dimerization domain (DD₂) comprises a CH3 domain comprising mutations D399N, E357Q, K439E and P352K in accordance with EU numbering.

In some embodiments, the first dimerization domain (DD₁) comprises a CH3 domain comprising mutations D399N, D/E356Q, K370E and L351A in accordance with EU numbering and the second dimerization domain (DD₂) comprises a CH3 domain comprising mutations D399N, E357Q, K439E and L351R in accordance with EU numbering. Accordingly, in some embodiments, the first dimerization domain (DD₁) comprises a CH3 domain comprising mutations D399N, D/E356Q, K370E and L351A in accordance with EU numbering. In some embodiments, the second dimerization domain (DD₂) comprises a CH3 domain comprising mutations D399N, E357Q, K439E and L351R in accordance with EU numbering.

In some embodiments, the first dimerization domain (DD₁) comprises a CH3 domain comprising mutations D399N, D/E356Q, K370E and P352Y in accordance with EU numbering and the second dimerization domain (DD₂) comprises a CH3 domain comprising mutations D399N, E357Q and K439E in accordance with EU numbering. Accordingly, in some embodiments, the first dimerization domain (DD₁) comprises a CH3 domain comprising mutations D399N, D/E356Q, K370E and P352Y in accordance with EU numbering. In some embodiments, the second dimerization domain (DD₂) comprises a CH3 domain comprising mutations D399N, E357Q and K439E in accordance with EU numbering.

In some embodiments, the first dimerization domain (DD₁) comprises a CH3 domain comprising mutations D399Q, D/E356Q, K370E, Y349K and S354K in accordance with EU numbering and the second dimerization domain (DD₂) comprises a CH3 domain comprising mutations D399N, E357Q and K439E in accordance with EU numbering. Accordingly, in some embodiments, the first dimerization domain (DD₁) comprises a CH3 domain comprising mutations D399Q, D/E356Q, K370E, Y349K and S354K in accordance with EU numbering. In some embodiments, the second dimerization domain (DD₂) comprises a CH3 domain comprising mutations D399N, E357Q and K439E in accordance with EU numbering.

In some embodiments, the first dimerization domain (DD₁) comprises a CH3 domain comprising mutations D399Q, D/E356Q, K370E, Y349K and S354K in accordance with EU numbering and the second dimerization domain (DD₂) comprises a CH3 domain comprising mutations D399N, E357Q, K439E and L351R in accordance with EU numbering. Accordingly, in some embodiments, the first dimerization domain (DD₁) comprises a CH3 domain comprising mutations D399Q, D/E356Q, K370E, Y349K and S354K in accordance with EU numbering. In some embodiments, the second dimerization domain (DD₂) comprises a CH3 domain comprising mutations D399N, E357Q, K439E and L351R in accordance with EU numbering.

In some embodiments, the protein complex may be composed of a first polypeptide comprising a first dimerization domain (DD₁) having any of the amino acid sequence disclosed herein and a second polypeptide comprising a second dimerization domain (DD₂) having any of the amino acid sequence disclosed herein.

In some embodiments, the first and second polypeptide of the protein complex each independently comprises in a N- to C-terminal fashion an amino acid sequence of formula Ia:

X-[(Abₐ)-(L_{b})]ₘ-(DD)-[(L_{c})-(Ab_{d})]ₙ-Y

Wherein m is 0, 1 or an integer greater than 1;

Wherein n is 0, 1 or an integer greater than 1, provided that m and n are not 0 simultaneously;

Wherein Abₐ, Ab_{d}, each independently comprise an antigen binding domain comprising one or more complementarity determining regions (CDRs) of an antibody;

Wherein X or Y are independently present or absent and comprises an amino acid sequence;

Wherein L_{b}, L_{c}, each independently comprises one or more linkers; and

Wherein DD is the first dimerization domain (DD₁) in the first polypeptide and the second dimerization domain (DD₂) in the second polypeptide.

In some embodiments, the first and second polypeptide each is independently a polypeptide disclosed herein.

In some embodiments, the first and second polypeptide each is independently a polypeptide having formula III and the dimerization domain is a natural dimerization domain.

In some embodiments, the first and second polypeptide each is independently a polypeptide having formula III and the dimerization domain is a mutated dimerization domain.

In some embodiments, the first polypeptide comprises formula II and the second polypeptide comprises formula III and the dimerization domain is a natural dimerization domain.

In some embodiments, the first polypeptide comprises formula II and the second polypeptide comprises formula III and the dimerization domain is a mutated dimerization domain.

In some embodiments the protein complex is multispecific.

In some embodiments, the protein complex is bispecific, trispecific or tetra specific.

In some embodiments, the first and second polypeptide of the protein complex have the same valency and specificity.

In some embodiments, the first and second polypeptide of the protein complex have different valency and specificity.

In some embodiments, the protein complex is a bispecific antibody and optionally the first and second polypeptide each is an antibody heavy chain.

In some embodiments, the bispecific antibody further comprises a first antibody light chain and second antibody light.

In further aspects and embodiments, the present disclosure relates to a composition comprising the protein complex disclosed herein.

In other aspects and embodiments, the present disclosure relates to a composition comprising monomers, dimers and mixture thereof.

In some embodiments, greater than 70%, 75%, 80%, 85%, 90%, 95%, 96%, 97%, 98%, or 99% of the first and second polypeptides exist as dimers in the composition.

In some embodiments, greater than 70%, 75%, 80%, 85%, 90%, 95%, 96%, 97%, 98%, or 99% of the first and second polypeptides exist as homodimers in the composition.

In some embodiments, greater than 70%, 75%, 80%, 85%, 90%, 95%, 96%, 97%, 98%, or 99% of the first and second polypeptides exist as heterodimers in the composition.

In an additional aspect and embodiments, the present disclosure relates to a method of treating a disorder or disease comprising administering the polypeptide disclosed herein.

In other aspects and embodiments, the present disclosure relates to a method of treating a disorder or disease comprising administering the protein complex disclosed herein.

In further aspects and embodiments, the present disclosure relates to a method of treating a disorder or disease comprising administering the composition of disclosed herein.

In some embodiments, the disorder or disease is cancer.

In some embodiments, the disorder or disease is an infection.

In some embodiments, the disorder or disease is immune dysregulation.

In other aspects and embodiments, the present disclosure relates to a method of making a protein complex, the method comprising transforming cells with one or more vectors comprising the nucleic acid disclosed herein.

In some embodiments, the method may further comprise isolating and/or purifying the polypeptide complex from impurities.

In other embodiments, the method may further comprise isolating and/or purifying heterodimers from monomers and/or homodimers.

In other embodiments, the method may further comprise isolating and/or purifying homodimers from monomers and/or heterodimers.

In further aspects and embodiments, the present disclosure relates to a kit comprising in same or separate vials one or more nucleic acids encoding a dimerization domain disclosed herein, one or more nucleic acids encoding an antigen binding domain and optionally one or more nucleic acids encoding a linker. In some embodiments, the dimerization domain is from a human antibody.

In some embodiments each nucleic acid is a vector.

In other embodiments each nucleic acid is a DNA segment comprising a unique overhang that allows assembly at a unique position into a DNA construct for encoding a polypeptide chain.

In some embodiments, one or more nucleic acids encoding a dimerization domain comprises a mutated CH3 domain of a human IgG1 having amino acid substitutions at position 356, 357, 370, 399 and/or 439 in accordance with EU numbering.

In some embodiments, the dimerization domain comprises a mutated CH3 domain of a human IgG1 having amino acid substitutions at position 356, 357, 370, 399 and/or 439 and further amino acid substitutions at positions corresponding to 349, 350, 351, 352, 354, 355, 394 and/or 395.

In some embodiments, the dimerization domain comprises a mutated CH3 domain of a human IgG1 having amino acid substitutions at position D/E356, E357, K370, D399 and/or K439 and further amino acid substitutions at positions corresponding to Y349, T350, L351, P352, S354, R355 or Q355, T394 and/or P395.

More particularly, in some exemplary embodiments, the dimerization domain comprises a mutated CH3 domain of a human IgG1 having amino acid substitutions at position D/E356, E357, K370, D399 and/or K439 and further amino acid substitutions at positions corresponding to Y349, T350, L351, P352, and/or S354.

In some embodiments, the nucleic acids each are DNA segments and the kit comprise in same or separate vials:
a) A DNA segment encoding a dimerization domain comprising a mutated CH3 domain of a human IgG1 having amino acid substitutions at position 356, 370 and 399 in accordance with EU numbering;
b) A DNA segment encoding a dimerization domain comprising a mutated CH3 domain of a human IgG1 having amino acid substitutions at position 357, 399 and 439;
c) one or more DNA segments encoding an antigen binding domain or antigen binding domains, and;
d) optionally one or more DNA segments encoding a linker or linkers;
wherein each nucleic acid is a DNA segment comprising a unique overhang that allows assembly at a unique position into a DNA construct for encoding a polypeptide chain.

In some embodiments, the kit is for assembly of a DNA construct encoding a polypeptide chain of formula Ia, formula Ib or formula Ic.

In other embodiments, the kit is for assembly of a DNA construct encoding a polypeptide chain of formula II, formula III, formula IV, formula V, formula VI, formula VII or formula VIII.

In other aspects and embodiments, the present disclosure relates to a method of making a nucleic acid encoding the polypeptide disclosed herein, the method comprises covalently assembling one or more DNA segments encoding a dimerization domain of a human antibody and one or more DNA segments encoding an antigen binding domain and optionally one or more DNA segments encoding a linker, wherein each DNA segment comprises a unique overhang that allow assembly at unique position into a DNA construct for encoding a polypeptide chain.

In some embodiments at least one DNA segment encodes a dimerization domain of a natural antibody.

In some embodiments at least one DNA segment encodes a mutated dimerization domain comprising a CH3 domain comprising amino acid substitutions that favorize heterodimer formation.

In some embodiments at least one DNA segment encodes a dimerization domain (DD) as described herein.

In some embodiments at least one DNA segment encodes a first dimerization domain (DD₁) having the amino acid sequence disclosed herein.

In some embodiments at least one DNA segment encodes a second dimerization domain (DD₂) having the amino acid sequence disclosed herein.

In some embodiments the amino acid substitutions comprises amino acid substitutions at positions 356, 370 and 399.

In some embodiments the amino acid substitutions comprises amino acid substitutions at positions 357, 399 and 439 in accordance with EU numbering.

In some embodiments, the DNA segment encodes a mutated dimerization domain comprising amino acid substitutions at positions 356, 370 and 399 and optionally further mutations at positions selected from 349, 350, 351, 352, 354, 355, 394 and/or 395. Accordingly, in some embodiments, the DNA segment encodes a mutated dimerization domain comprising an amino acid substitution at position 356. In some embodiments, the DNA segment encodes a mutated dimerization domain comprising an amino acid substitution at position 370. In some embodiments, the DNA segment encodes a mutated dimerization domain comprising amino acid substitutions at position 399. In some embodiments, the DNA segment encodes a mutated dimerization domain comprising amino acid substitutions at positions 356, 370 and 399. In some embodiments, the DNA segment encodes a mutated dimerization domain further comprising a mutation a position 349. In some embodiments, the DNA segment encodes a mutated dimerization domain further comprising a mutation a position 350. In some embodiments, the DNA segment encodes a mutated dimerization domain further comprising a mutation a position 351. In some embodiments, the DNA segment encodes a mutated dimerization domain further comprising a mutation a position 352. In some embodiments, the DNA segment encodes a mutated dimerization domain further comprising a mutation a position 354. In some embodiments, the DNA segment encodes a mutated dimerization domain further comprising a mutation a position 355. In some embodiments, the DNA segment encodes a mutated dimerization domain further comprising a mutation a position 394. In some embodiments, the DNA segment encodes a mutated dimerization domain further comprising a mutation a position 395.

In some embodiments, the DNA segment encodes a mutated dimerization domain comprising amino acid substitutions at positions 357, 399 and 439 and optionally further mutations at positions selected from 349, 350, 351, 352, 354, 355, 394 and/or 395.

Accordingly, in some embodiments, the DNA segment encodes a mutated dimerization domain comprising an amino acid substitution at position 357. In some embodiments, the DNA segment encodes a mutated dimerization domain comprising an amino acid substitution at position 399. In some embodiments, the DNA segment encodes a mutated dimerization domain comprising an amino acid substitution at positions 439. In some embodiments, the DNA segment encodes a mutated dimerization domain comprising amino acid substitutions at positions 357, 399 and 439. In some embodiments, the DNA segment encodes a mutated dimerization domain further comprising a mutation at position 349. In some embodiments, the DNA segment encodes a mutated dimerization domain further comprising a mutation at position 350. In some embodiments, the DNA segment encodes a mutated dimerization domain further comprising a mutation at position 351. In some embodiments, the DNA segment encodes a mutated dimerization domain further comprising a mutation at position 352. In some embodiments, the DNA segment encodes a mutated dimerization domain further comprising a mutation at position 354. In some embodiments, the DNA segment encodes a mutated dimerization domain further comprising a mutation a position 355. In some embodiments, the DNA segment encodes a mutated dimerization domain further comprising a mutation a position 394. In some embodiments, the DNA segment encodes a mutated dimerization domain further comprising a mutation a position 395. In some embodiments, the nucleic acid comprises at least two DNA segments encoding an antigen binding domain.

In some embodiments, the nucleic acid comprises at least three DNA segments encoding an antigen binding domain.

In some embodiments, the nucleic acid comprises at least four DNA segments encoding an antigen binding domain.

In some embodiments, the nucleic acid comprises at least one DNA segment encoding an antigen binding domain at each of the 5′- and 3′-end of a DNA segment encoding a dimerization domain.

In other aspects and embodiments, the present disclosure relates to a method of making the polypeptide or the protein complex disclosed herein, the method comprising transforming a cell with a nucleic acid made by a method comprising covalently assembling one or more DNA segments encoding a dimerization domain of a human antibody and one or more DNA segments encoding an antigen binding domain and optionally one or more DNA segments encoding a linker, wherein each DNA segment comprises a unique overhang that allow assembly at unique position into a DNA construct for encoding a polypeptide chain.

In some embodiments, the one or more of the DNA segments encodes a dimerization domain comprising a) a mutated CH3 domain of a human IgG1 having amino acid substitutions at position 356, 370 and 399 in accordance with EU numbering or b) a mutated CH3 domain of a human IgG1 having amino acid substitutions at position 357, 399 and 439.

In some embodiments, one DNA segment encodes a dimerization domain comprising a mutated CH3 domain of a human IgG1 having amino acid substitutions at position 356, 370 and 399 in accordance with EU numbering and another DNA segment encodes a mutated CH3 domain of a human IgG1 having amino acid substitutions at position 357, 399 and 439.

In some embodiments the mutated CH3 domain comprises further amino acid substitutions at one or more positions selected from 349, 350, 351, 352, 354, 355, 394 and/or 395. Accordingly, in some embodiments, the mutated CH3 domain comprises further amino acid substitutions at position 349. In some embodiments, the mutated CH3 domain comprises further amino acid substitutions at position 350. In some embodiments, the mutated CH3 domain comprises further amino acid substitutions at position 351. In some embodiments, the mutated CH3 domain comprises further amino acid substitutions at position 352. In some embodiments, the mutated CH3 domain comprises further amino acid substitutions at position 354. In some embodiments, the DNA segment encodes a mutated dimerization domain further comprising a mutation a position 355. In some embodiments, the DNA segment encodes a mutated dimerization domain further comprising a mutation a position 394. In some embodiments, the DNA segment encodes a mutated dimerization domain further comprising a mutation a position 395.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### BRIEF DESCRIPTION OF THE DRAWINGS

FIG. 1: schematics representing the modular design and assembly of exemplary multivalent proteins disclosed herein. VHHs may be selected and combined with linkers and dimerization domains to form multivalent and/or multispecific polypeptides dimers.

FIG. 2A-2B: schematics representing exemplary configurations of polypeptides comprising three VHH domains shown as a monomer (FIG. 2A) or protein dimers (FIG. 2B) in either the symmetrical homodimer form or the heterodimer form of the present disclosure.

FIGS. 3A-3C: pictures of 4-12% Bis-Tris gradient SDS-PAGE gels performed under reducing conditions loaded with supernatants containing polypeptides KB001 (FIG. 3A), KB003 and KB004 (FIG. 3B), or KB005 (FIG. 3C). A serial dilution of bovine serum albumin (BSA) was used a loading control. Gels were stained using GelCode™ staining reagent to visualize the proteins.

FIG. 4A-4B: pictures of 4-12% Bis-Tris gradient SDS-PAGE gels performed under reducing conditions loaded with supernatants containing polypeptides KB007 and KB008 (FIG. 4A), or KB009 and KB010 (FIG. 4B) expressed in mammalian cells. A serial dilution of bovine serum albumin (BSA) was used a loading control. Gels were stained using GelCode™ staining reagent to visualize the proteins.

FIGS. 5A and 5B: picture of 4-12% Bis-Tris gradient SDS-PAGE gels loaded with supernatants containing polypeptides KB012, KB013, and KB011 under non-reducing conditions using a 4-12% Bis-Tris gradient SDS-PAGE gel (FIG. 5A). A serial dilution of bovine serum albumin (BSA) was used a loading control. Picture of 8% Tris-Glycine SDS-PAGE gel loaded with 2 μg KB012, KB013, and KB011 run under non-reducing and reducing conditions (FIG. 5B). Gels were stained using GelCode™ staining reagent to visualize the proteins.

FIGS. 6A and 6B: pictures of 8% Tris-Glycine SDS-PAGE gels performed with samples containing 2 μg of purified polypeptides KB001, KB003, KB004 or KB005, under non-reducing conditions (FIG. 6A) or under reducing conditions (FIG. 6B). Gels were stained using GelCode™ staining reagent to visualize the proteins.

FIG. 6C: table summarizing the production yield, the isoelectric point (pI) and molecular weight (MW) of polypeptides KB001, KB003, KB004 or KB005.

FIGS. 6D and 6E: pictures of 8% Tris-Glycine SDS-PAGE gels loaded with 2 μg of purified polypeptides KB008, KB009 or KB007 under non-reducing conditions (FIG. 6D) or under reducing conditions (FIG. 6E). Gels were stained using GelCode™ staining reagent to visualize the proteins.

FIG. 6F: table summarizing the production yield, isoelectric point (PI) and molecular weight (MW) of polypeptides KB008, KB009 or KB007.

FIG. 7: histogram representing flow cytometry binding data of dimers made from the KB017 (negative control), KB019, or KB015 polypeptides to Jurkat cells.

FIG. 8A-8B: schematic representing homodimers obtained from the KB019 (FIG. 8A) and KB015 polypeptides (FIG. 8B).

FIGS. 8C-8D: graphs representing data of PBMC-dependent cytotoxicity assays performed by incubation of human PBMCs with OCI-AML3 and dimers made from the KB017 and KB019 polypeptides (FIG. 8C) or with dimers made from the KB017, KB019 and KB015 polypeptides for 48 hours (FIG. 8D).

FIG. 9A: graph representing data of PBMC-dependent cytotoxicity assays performed on OCI-AML3 cells with dimers made from the KB015, KB016 or KB018 polypeptides assayed at 48 hours.

FIG. 9B: graph representing data of cytotoxicity assays by incubation of human PBMCs with OCI-AML3 cells in the presence of dimers made from the KB074, KB075, KB076 and KB078 polypeptides for 48 hours.

FIG. 10: graph representing data of cytotoxicity assays performed on THP-1 cells with dimers made from the KB020, KB021, KB022, KB015, KB023 polypeptides or with a combination of the antibody-Fc fusions KB045, KB046 and KB033.

FIG. 11A: schematic showing position selected for linker modification.

FIG. 11B-11C: histogram and graphs showing a binding curve of the different protein dimers to human recombinant protein PD-1.

FIG. 12A: schematic showing position selected for linker modification.

FIG. 12B-12C: histogram and graphs showing binding of the different protein dimers to recombinant protein PD-1.

FIG. 13A: graph representing binding of dimers made from the KB001, KB003, KB004, KB005 or KB017 polypeptides to DRD2 proteoliposomes or to empty liposomes.

FIG. 13B: graph representing binding of dimers made from the KB007, KB008, KB009 or KB017 polypeptides to DRD1 proteoliposomes or to empty liposomes.

FIG. 14A-14B: histogram showing the viability of NCI-H510 cells (FIG. 14A) or NCI-H69 cells (FIG. 14B) incubated with human PBMCs at a ratio of 1:10, and with dimers made from the KB015, KB018, KB001, KB003, KB004 or KB005 polypeptides at a concentration of 10 μg/mL.

FIG. 14C: histogram showing viability of NCI-H510 cells incubated with human PBMCs at a ratio of 1:10, and with dimers made from the KB015, KB018, KB007 or KB008 polypeptides at a concentration of 10 μg/mL.

FIG. 15A-15B: graph representing data of human PBMC-dependent cytotoxicity assays performed on OCI-AML3 with dimers made from the KB017, KB019, KB012 or KB013 polypeptides (FIG. 15A) or with dimers made from the KB011, KB015, KB017, KB012, KB013 or KB014 polypeptides for 48 hours (FIG. 15B).

FIG. 16A: graph representing tumor volume over time of NOG mice injected subcutaneously with OCI-AML3 tumor and human PBMCs and treated with dimers made from the KB015, KB017 or KB019 polypeptides or with PBS at 28 mg/kg by intraperitoneal (i.p.), once a week.

FIG. 16B: graph representing tumor volume over time in NOG mice injected subcutaneously with OCI-AML3 tumor and human PBMCs and treated with dimers made from the KB017, KB011 polypeptides or with KB058 or with PBS at 28 mg/kg, once a week.

FIG. 17: graph representing tumor progression in SCID mouse xenografts of small cell lung cancer NCI-H510A model treated with KB120 or negative control sdAb (NC) at 16 mg/kg for once a week.

FIG. 18A: graph representing binding of protein complexes that comprise an anti-PD-1 VHH to human PD-1 (KB072) compared to positive control or negative control antibodies.

FIG. 18B: graph representing CPI function (immune checkpoint inhibition) of protein complexes that comprise an anti-PD-1 VHH to human PD-1 (KB072) compared to positive control or negative control antibodies.

FIG. 19: graph showing tumor progression in the NCI-H82 SCLC human PBMC co-engraftment model treated with protein complexes comprising VHHs that target DRD2, PD1 and T-cells (KB073) or with negative control antibody at the dose of 28 mg/kg, biweekly, for a total of eight doses.

FIG. 20A: schematic representing homodimers made from the KB047 polypeptide.

FIG. 20B: graph representing data of cytotoxicity assays performed on OCI-AML3 cells with dimers made from the KB047, KB015, KB018 or KB048 polypeptides.

FIG. 21A: picture of Western blot performed following SDS-PAGE analysis of dimers made by co-transfecting cells with different ratios of DNA encoding the KB049 polypeptide lighter chain (lane 1), the KB050 polypeptide heavier chain (lane 2) or co-transfected with both plasmids (identified as KB057) at ratios of 1:1, 3:1, and 1:3 (lanes 3 to 5).

FIG. 21B: table summarizing the molecular weight of homodimers made from the KB050 or KB049 polypeptides or heterodimers made from KB057 co-transfection of KB049 and KB050.

FIG. 22A: table summarizing the molecular weight of monomers, homodimers or heterodimers made by co-transfecting cells with DNA constructs expressing Chain A and Chain B of the KB051, KB052, KB053 or KB054 polypeptides, and DNA ratios used for co-transfection of chain a and chain B.

FIGS. 22B-22C: picture of Western blot performed following SDS-PAGE done under non-reducing (FIG. 22B) or reducing (FIG. 22C) and loaded with protein dimers made by co-transfecting cells with a 1:1 ratio of DNA constructs expressing Chain A and Chain B of the KB051 (lane 1), KB052 (lane 2), KB053 (lane 3) or a 1:2 ratio of DNA constructs expressing Chain A and Chain B KB054 (lane 4) polypeptides.

### DETAILED DESCRIPTION

### Definitions

Unless indicated otherwise, the amino acid numbering indicated for the dimerization domain are in accordance with the EU numbering system.

The use of the terms “a” and “an” and “the” and similar referents in the context of describing embodiments (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

The term “treatment” for purposes of this disclosure refers to both therapeutic treatment and prophylactic or preventative measures. Those in need of treatment include those already with the disorder as well as those prone to have the disorder or those in whom the disorder is to be prevented.

The term “about” or “approximately” with respect to a given value means that variation in the value is contemplated. In some embodiments, the term “about” or “approximately” shall generally mean a range within +/−20 percent, within +/−10 percent, within +/−5, +/−4, +/−3, +/−2 or +/−1 percent of a given value or range.

The term “functionally active” with reference to an antigen binding domain means that the antigen binding domain is capable of binding to its target and optionally that the antigen binding domain possesses one or more biological activities.

As used herein the term “flexible linker” refers to peptide comprising at least a portion composed of flexible amino acid residues that allow adjacent modules to move relative to one another.

As used herein the term “rigid linker” refers to peptide comprising at least a portion composed of amino acids that exhibit a rigid structure and that keeps a distance between two modules.

As used herein the term “helical linker” means a linker that is composed of amino acid residues that adopt a α-helical conformation.

As used herein the term “cleavable linker” refers to peptides that comprise an enzymatic cleavage site that is sensitive to proteases selected from the group consisting of ADAMS, ADAMTS, aspartate proteases, caspases, cysteine cathepsins, cysteine proteinases, metalloproteinases, serine proteases, coagulation factor proteases, Type II Transmembrane Serine Proteases (TTSPs) and combination thereof.

It is to be understood herein, that expressions referring to ranges of values in the format such as “from A to B”, include each individual value and any sub-range comprised and including such ranges. For example, the expression “from 1 to 10” includes sub-ranges such as and without limitations, “from 2 to 10”, “from 2 to 9”, “from 3 to 6”, “from 5 to 7” and any individual values comprised between and including 1 and 10, i.e., 1, 2, 3, 4, 5, 6, 7, 8, 9 or 10.

It is to be understood herein that the term “at least” with respect to a given value intends to include the value and superior values. For example, the term “at least 80%” include “at least 81%”, “at least 82%”, “at least 83%”, “at least 84%”, “at least 85%”, “at least 86%”, “at least 87%”, “at least 88%”, “at least 89%”, “at least 90%”, “at least 91%”, “at least 92%”, “at least 93%”, “at least 94%”, “at least 95%”, “at least 96%”, “at least 97%”, “at least 98%”, “at least 99%”, “at least 99.1%”, “at least 99.2%”, at least 99.3%”, at least 99.4%”, at least 99.5%”, at least 99.6%”, at least 99.7%”, at least 99.8%”, at least 99.9%”, and 100%.

### Polypeptides

Segments of DNA encoding desired polypeptide sequences are synthesized in vitro. The different DNA modules are assembled into a single piece in an organized and directional manner which is then cloned into an expression vector. The resulting polypeptides are therefore composed of different modules forming a single chain.

The polypeptides of the present disclosure include, for example and without limitation, antigen binding domains, linkers and a dimerization domain that promote assembly of at least two polypeptide chains.

In an exemplary configuration, one or more antigen binding domains may be located at the N-terminus, at the C-terminus or on each side of the dimerization domain.

In another exemplary configuration, the polypeptide may comprise at least one antigen binding domain at the N-terminus of the dimerization domain and at least one antigen binding domain at the C-terminus of the dimerization domain.

In a further exemplary configuration, the polypeptide may comprise one antigen binding domain at the N-terminus of the dimerization domain and at least two antigen binding domains at the C-terminus of the dimerization domain.

In yet a further exemplary configuration, the polypeptide may comprise two antigen binding domains at the N-terminus of the dimerization domain and two antigen binding domains at the C-terminus of the dimerization domain.

In some embodiments, the polypeptide may comprise in a N- to C-terminal fashion an amino acid sequence having the configuration set forth in formula Ia:

X-[(Abₐ)-(Lb)]ₘ-(DD)-[(L_{c})-(Ab_{d})]ₙ-Y

Wherein m may be 0, 1 or an integer greater than 1;
Wherein n may be 0, 1 or an integer greater than 1, provided that m and n are not 0 simultaneously;
Wherein Abₐ, Ab_{d}, each may independently comprise an antigen binding domain comprising one or more complementarity determining regions (CDRs) of an antibody;
Wherein X or Y may independently be present or absent and may comprise an amino acid sequence;
Wherein L_{b}, L_{c}, may each independently comprise one or more linkers; and
Wherein DD comprises a dimerization domain.

In some embodiments, the polypeptide may comprise in a N- to C-terminal fashion an amino acid sequence having the configuration set forth in formula Ib:

X-[(Abₐ)-(L_{b})]ₘ-(DD)-[(L_{c})-(Ab_{d})]ₙ-Y

Wherein m may be 0, 1 or an integer greater than 1;
Wherein n may be 2 or an integer greater than 2;
Wherein Abₐ, Ab_{d}, may each independently comprise an antigen binding domain comprising one or more complementarity determining regions (CDRs) of an antibody;
Wherein X or Y may independently be present or absent and may comprise an amino acid sequence;
Wherein L_{b}, L_{c}, may each independently comprise one or more linkers;
Wherein L_{c} does not comprise a cleavable linker; and
Wherein DD may comprise a dimerization domain.

In exemplary embodiments m may be 2. In other exemplary embodiments, m may be 3. In further exemplary embodiments, m may be 4. In additional exemplary embodiments, m may be 5. In other exemplary embodiments, m may be greater than 5.

In exemplary embodiments n may be 2. In other exemplary embodiments, n may be 3. In further exemplary embodiments, n may be 4. In additional exemplary embodiments, n may be 5. In other exemplary embodiments, n may be greater than 5.

It is to be understood herein that in formula Ia, formula Ib or formula Ic disclosed herein, when m is 2 or an integer greater than 2, each of Abₐ, L_{b} or each unit defined by (Abₐ)-(L_{b}) may be the same or different.

It is to be understood herein that in formula Ia, formula Ib or formula Ic disclosed herein, when n 2 or an integer greater than 2, each of Ab_{d}, L_{c} or each unit defined by (L_{c})-(Ab_{d}) may be the same or different.

Embodiments of polypeptides include, for example and without limitations, those having the configuration set forth in formula II, the configuration set forth in formula III, the configuration set forth in formula IV, the configuration set forth in formula V, the configuration set forth formula VI, the configuration set forth in formula VII, the configuration set forth in formula VIII

X-(Abₐ₁)-(L_{b1})-(DD)-(L_{c1})-(Ab_{d1})-Y
(formula II);
X-(Abₐ₁)-(L_{b1})-(DD)-(L_{c1})-(Ab_{d1})-(L_{c2})-(Ab_{d2})-Y
(formula III);
X-(Abₐ₁)-(L_{b2})-(Abₐ₂)-(L_{b1})-(DD)-(L_{c1})-(Ab_{d1})-Y
(formula IV);
X-(Abₐ₁)-(L_{b2})-(A_{ba2})-(L_{b1})-(DD)-(L_{c1})-
(Ab_{d1})-(L_{c2})-(Ab_{d2})-Y
(formula V)
X-(Abₐ₁)-(L_{b2})-(Abₐ₂)-(L_{b1})-(DD)-(L_{c1})-(Ab_{d1})-
(L_{c2})-(Ab_{d2})-(L_{c3})-(Ab_{d3})-Y
(formula VI);
X-(Abₐ₁)-(L_{b3})-(Abₐ₂)-(L_{b2})-(Abₐ₃)-(L_{b1})-(DD)-
(L_{c1})-(Ab_{d1})-(L_{c2})-(Ab_{d2})-Y
(formula VII);
X-(Abₐ₁)-(L_{b3})-(Abₐ₂)-(L_{b2})-(Abₐ₃)-(L_{b1})-(DD)-
(L_{c1})-(Ab_{d1})-(L_{c2})-(Ab_{d2})-(L_{c3})-(Ab_{d3})-Y
(formula VIII);

Wherein X, Y and DD are as defined in formula Ia, formula Ib or formula Ic;
Wherein Abₐ₁, Abₐ₂, Abₐ₃, Ab_{d1}, Ab_{d2}, Ab_{d3}, each independently comprise an antigen binding domain comprising one or more complementarity determining regions (CDRs) of an antibody;
Wherein L_{b1} comprises a linker or linkers and/or a hinge region of an antibody or antigen binding fragment thereof and;
Wherein L_{b2}, L_{b3} L_{c1}, L_{c2}, and L_{c3} each independently comprise a linker or linkers.

The polypeptides of the present disclosure comprise antigen binding domains that are functionally active either as a single chain or when part of the protein complex disclosed herein.

For example, the antigen binding domain of the polypeptide may bind to its target and may biologically active.

In some embodiments, the biological activity of an antigen binding domain includes, for example and without limitation, blocking binding of a target to its natural receptor or ligand.

Alternatively, the biological activity of an antigen binding domain includes its ability to sequester a target. Moreover, the biological activity of an antigen binding domain includes its ability to induce signalling.

A polypeptide that comprises more than one antigen binding domain is characterized as being multivalent.

The polypeptide of the present disclosure may comprise an additional amino acid sequence at its N- or C-terminus or at both ends (defined by X and Y respectively in the formulas disclosed herein).

In some embodiments, the amino acid sequence at the N-terminus (defined by X) may include a signal peptide, an exemplary embodiment of which is provided in SEQ ID NO:51.

In some embodiments, the amino acid sequence at the N-terminus (defined by X) or C-terminus (defined by Y) may independently include a linker, a cytokine, a chemokine, a tag (e.g., His tag (e.g. SEQ ID NO:52), a masking domain, a phage coat protein, an antigen binding domain or combination thereof.

### Antigen Binding Domains (Abs)

The polypeptides of the present disclosure comprise one or more antigen binding domains each independently comprising one or more complementarity determining region(s) (CDRs) of an antibody.

The specificity of the polypeptides of the present disclosure may thus be conferred by their antigen binding domains.

In some embodiments, all antigen binding domains of a given polypeptide chain are capable of binding to their targets when combined as a single chain with the different modules.

The polypeptides of the present disclosure may comprise antigen binding domains derived from a natural antibody (of human or animal origin) or from a synthetic antibody.

In some embodiments, antigen binding domains of a natural antibody are engineered so as to form a single chain.

In some embodiments, antigen binding domains may be obtained from IgGs such as IgG1, IgG2, IgG3 or IgG4. In particular embodiments, antigen binding domains are derived from a human IgG heavy chain.

In some embodiments, the antigen binding domains may be obtained from heavy chain only antibodies (HCAbs).

Exemplary embodiments of antigen binding domains include for example and without limitation a single domain antibody (sdAb), a heavy chain variable region (VH or VHH), a light chain variable region (VL or VLL), a single chain variable fragment (scFv), a V_{NAR} fragment, and combinations thereof.

In a particular embodiment, the polypeptides of the present disclosure may comprise an antigen binding domain VHH derived from humans or a mouse or rat or from a transgenic mouse or rat wherein a mouse or rat VHH has been camelized, a human VHH, a human VHH which has been camelized, of an IgG1, IgG2a, IgG2b, IgG2c or IgG3 or combination thereof. The antibodies may be obtained by immunizing a mouse or a rat or a transgenic mouse or rat which is lacking a functional CH1 domain in any of its heavy chains, IgG1, IgG2a, IgG2b, IgG2c or IgG3 or combination thereof, or a combination of the VHH described above, with an antigen of interest.

In a particular embodiment, the polypeptides of the present disclosure may comprise an antigen binding domain of a camelid antibody such as VHH of an IgG2 or IgG3. The camelid antibodies may be obtained by immunizing a dromedary, a camel, a llama or an alpaca with an antigen of interest.

In some embodiments, the camelid antibodies may originate from the so-called old-world camelids such as Camelus bactrianus, Camelus dromaderus or from new-world camelids such as Lama pacos, Lama glama and Lama vicugna.

In another particular embodiment, the polypeptides of the present disclosure may comprise an antigen binding domain of a cartilaginous fish such as a V_{NAR} fragment of IgNAR. The V_{NAR} fragment may originate from shark antibodies.

If desired, the antigen binding domain of a non-human antibody may be humanized. For example, the framework region of non-human VH, VHH or HCAbs may be modified so as to render them more human-like. Humanization of camelid antibodies is discussed for example in Vincke C. et al. (J. Biol Chem. 2009, 284(5):3273-3284), the entire content of which is incorporated herein by reference. Humanized camelid antibodies may be obtained, for example, by CDR grating onto a universal humanized nanobody scaffold (e.g., h-NbBcII10_{FGLA} disclosed in Vincke C. et al.). V_{NAR} antibodies can be humanized by converting non-CDR residues to those of human germline V□1 sequence DPK9 as discussed in Kovalenko O V et al. (J Biol Chem. 2013, 288:17408-17419) the entire content of which is incorporated herein by reference. The polypeptides of the present disclosure therefore encompass humanized antigen binding domains.

In yet another particular embodiment, the antigen binding domain may comprise a human VH (modified or not). Human VH may be obtained for example, from synthetic human VH libraries. Modified human VH include those in which some amino acid residues have been modified to render them more camel-like (i.e., by camelization).

In some aspects of the disclosure, the polypeptide may be composed of antigen binding domains that all bind to the same target and to the same epitope. Such polypeptide may be characterized as being monospecific.

An exemplary embodiment of a monospecific polypeptide includes a polypeptide that comprise antigen binding domains having identical CDRs and framework regions. Another exemplary embodiment of a monospecific polypeptide includes, a polypeptide comprising antigen binding domains that have identical CDRs and different framework regions. A further exemplary embodiment of a monospecific polypeptide includes a polypeptide that comprise antigen binding domains that differ in the amino acid sequence of one or more of their CDRs (e.g., conservative substitution in one or more CDRs) without affecting their ability to bind to the same epitope or antigen.

In other aspects of the disclosure, the antigen binding domains of the polypeptide may bind to different epitopes of the same antigen or to different antigens. Such polypeptides may be characterized as being multispecific and encompass for example, bispecific polypeptides, trispecific polypeptides, tetraspecific polypeptides, pentaspecific polypeptides, hexaspecific polypeptides, biparatopic polypeptides, multiparatopic polypeptides and the like.

An exemplary embodiment of a multispecific polypeptide include a polypeptide that comprises at least two antigen binding domains that differ in the amino acid sequence of one or more of their CDRs leading to different binding specificities.

A polypeptide may more particularly be characterized as being bispecific when it binds to two different epitopes or antigens. A polypeptide may be characterized as being trispecific when it binds to three different epitopes or antigens. A polypeptide may be characterized as being tetraspecific when it binds to four different epitopes or antigens. A polypeptide may be characterized as being pentaspecific when it binds to five different epitopes or antigens. A polypeptide may be characterized as being hexaspecific when it binds to six different epitopes or antigens.

A polypeptide comprising two antigen binding domains that bind to two non-overlapping epitopes on the same target is characterized as being biparatopic. A polypeptide comprising antigen binding domains that bind to three, four or more epitopes on the same target is characterized as being multiparatopic.

The antigen binding domains of a given polypeptide will be selected based on the intended use such as detection, diagnostic and/or therapeutic use. Each of the antigen binding domains of a particular polypeptide may be selected so as to generate an additive or synergic effect.

In some embodiments the antigen binding domain may be selected for its ability to specifically binds a protein involved in a disease or condition.

For example, polypeptides of the present disclosure may comprise at least one antigen binding domain that specifically binds to an antigen expressed by tumor cells or by the tumor cell environment (i.e., tumor-specific antigen binding domains).

The polypeptides of the present disclosure may thus comprise one or more antigen binding domains that specifically binds to CD36, DRD1, DRD2, DRD3, DRD4, DRD5, PD-L1, TROP2, CD147, MCT1, IL1RAP, AMIGO2, PTK7, MCT2, MCT4, NHE1, H+/K+-ATPase, LAP, HLA-I A2, CD73, CD98, CEA, CEACAM5/6, ICAM-1, MCSP, fibronectin, Beta 1 Integrin, Tetraspanin 8, CD164, CD59, CD63, CD44, CD166, cWF, TNF, IL-17A, IL17-F, IL-6R, BCMA, TNF, RANKL, ADAMTS5, VEGF, Ang2, CX₃CR1, CXCR4, CXCR7, CXCL12, TfR1 (CD71), CXCR2, VEGFR2, CD19, IGFR1, EpCAM, EGFR, DLL3, CGRP, CD79b, CD28, CCR5, ErbB3, ErbB2, TGFβ1, TGFβ2, TGFβ3, TGFβR1, TGFβR2, IDO1, IDO2, TLR-4, TLR-7, TLR-8, TLR-9 etc.

In some embodiments, the antigen binding domain may specifically bind to a receptor.

In some embodiments, the antigen binding domain may specifically bind to a G-protein coupled receptor, such as for example and without limitations, a dopamine receptor.

In some exemplary embodiment, the dopamine receptor may be dopamine receptor D1 (DRD1).

In some exemplary embodiment, the dopamine receptor may be dopamine receptor D2 (DRD2).

In some exemplary embodiment, the dopamine receptor may be dopamine receptor D3 (DRD3).

In some exemplary embodiment, the dopamine receptor may be dopamine receptor D4 (DRD4).

In some exemplary embodiment, the dopamine receptor may be or dopamine receptor D5 (DRD5).

In other aspects and embodiments of the disclosure the polypeptides may comprise at least one antigen binding domain that specifically binds to an immunomodulator.

For example, the polypeptide may comprise one or more antigen binding domains that bind an immune checkpoint protein, a cytokine, a chemokine or an immune receptor or coreceptor etc (e.g., immune-specific antigen binding domains).

The polypeptides of the present disclosure may thus comprise one or more antigen binding domains that specifically binds to CD3, CD16, PD1, PDL-1, CTLA-4, CD8, LAG-3, OX40, CD27, CD122/IL2RB, TLR8/CD288, TIM-3, ICOS/CD278, NKG2A, A2AR, B7-H3, GITR/TNFRSF18, 4-IBB/CD137, KIR2DL1, KIR3DL2, SIRPα, CD47, VISTA, CD40, CD112, CD96, TOGOT, BTLA, TIGIT, LAG-3, CD4 etc.

In an exemplary embodiment, the polypeptide of the present disclosure may comprise at least one tumor-specific antigen binding domain and at least one immune-specific antigen binding domain.

Several single domain antibodies have been described in the literature. The polypeptides of the present disclosure may thus comprise an antigen binding domain that comprise the CDRs, full sequence of such single domain antibodies.

Exemplary embodiments of such single domain antibodies include, without limitations, those that target CXCR2 (U.S. Pat. No. 9,328,174 B2 (2016), U.S. Pat. No. 9,688,763B2 (2017)), CXCR4 (U.S. Pat. No. 9,212,226 B2 (2015)), CXCR7 (U.S. Pat. No. 9,212,226 B2 (2015), U.S. Pat. No. 8,937,164B2 (2015), U.S. Pat. No. 9,758,584B2 (2017), U.S. Pat. No. 999,639B2 (2018)), C-MET (U.S. Pat. No. 8,703,135 B2 (2014), U.S. Pat. No. 9,346,884 B2 (2016), U.S. Pat. No. 9,683,045B2 (2017)), KRAS (U.S. Pat. No. 9,663,570B2 (2017)), TNF-alpha (U.S. Pat. No. 9,546,211B2 (2017), U.S. Pat. No. 8,703,131B2 (2014), U.S. Pat. No. 9,067,991B2 (2015), U.S. Pat. No. 9,371,381B2 (2016), U.S. Pat. No. 9,745,372 B2 (2017)), serum albumin (U.S. Pat. No. 8,217,140B2 (2012), U.S. Pat. No. 8,188,223 B2 (2012), U.S. Pat. No. 9,573,992 B2 (2017)), von Willebrand Factor (U.S. Pat. No. 7,807,162 B2 (2010), U.S. Pat. No. 8,372,398 B2 (2013), U.S. Pat. No. 9,028,816B2 (2015), U.S. Ser. No. 10/112,989B2 (2018)), RANK-L (U.S. Pat. No. 8,623,361 B2 (2014), U.S. Pat. No. 9,475,877 B2 (2016), U.S. Pat. No. 9,505,840 B2 (2016), U.S. Pat. No. 9,534,055 B2 (2017)), IL-6R (U.S. Pat. No. 8,629,244 B2 (2014), U.S. Pat. No. 8,748,581 B2 (2014), U.S. Pat. No. 8,962,805B2 (2015), U.S. Pat. No. 9,181,350 B2 (2015), U.S. Pat. No. 9,273,150 B2 (2016), U.S. Pat. No. 9,605,072 B2 (2017), U.S. Pat. No. 9,611,326 B2 (2017), U.S. Pat. No. 9,617,341 B2 (2017), U.S. Ser. No. 10/118,967B2 (2018)), OX40L (U.S. Pat. No. 8,962,807B2 (2015), U.S. Pat. No. 9,834,611B2 (2017)), HER2 (U.S. Pat. No. 8,975,382B2 (2015), U.S. Pat. No. 9,969,805B2 (2018)), HER3 (U.S. Pat. No. 9,932,403B2 (2018)), IL-17A and IL-17F (U.S. Ser. No. 10/017,568B2 (2018)), EGFR (U.S. Pat. No. 9,243,065 B2 (2016)), STAT3 (U.S. Pat. No. 9,695,234B2 (2017)), amyloid Beta (U.S. Pat. No. 9,211,330B2 (2015)), Pseudomonas (U.S. Ser. No. 10/072,098 B2 (2018)), P2X7 receptor (U.S. Pat. No. 9,908,935B2 (2018)), Hepatocyte Growth factor (U.S. Pat. No. 9,670,275B2 (2017), U.S. Ser. No. 10/100,110 B2 (2018)), Notch pathway members (U.S. Pat. No. 8,557,965 B2 (2013)), angiopoletin/Tie (U.S. Pat. No. 8,858,940B2 (2014), U.S. Pat. No. 9,382,333B2 (2016), U.S. Pat. No. 9,822,175 B2 (2017)), chemokines (U.S. Pat. No. 8,906,680 B2 (2014)), G-coupled protein receptors (U.S. Pat. No. 9,512,236 B2 (2016), scavenger receptors (U.S. Pat. No. 9,034,325B2 (2015)), intracellular antigens (U.S. Pat. No. 9,850,321B2 (2017)), metalloproteinases (U.S. Pat. No. 9,156,914B2 (2015)) etc.

Specific exemplary embodiments of such single domain antibodies include those that are part of Caplacizumab (VHH against vWF), Ozoralizumab (VHH against TNF), ALX/0761/M1095 (VHH bispecific against IL-17A, I-17F), Vobarilizumab (VHH against IL-6R), LCAR-B38M (VHH against BCMA), V565 (VHH against TNF), ALX-1141/M6495 (VHH against ADAMTSS), BI 836880 (VHH bispecific against VEGF, Ang2), BI 655088 (VHH against CX₃CR1), AD-214 (i-body against CXCR4), TXB4 (VNAR against TfR1), ALX-0141 (VHH against RANK-L) etc.

In some embodiments, the polypeptide disclosed herein may comprise one or more tumor-specific antigen binding domains at the N-terminus of the dimerization domain.

In some embodiments, the polypeptide disclosed herein may comprise one or more tumor-specific antigen binding domains at the C-terminus of the dimerization domain.

In some embodiments, the polypeptide disclosed herein may comprise one or more tumor-specific antigen binding domains at both the N- and C-terminus of the dimerization domain.

In some embodiments, the polypeptide disclosed herein may comprise one or more immune-specific antigen binding domains at the N-terminus of the dimerization domain.

In some embodiments, the polypeptide disclosed herein may comprise one or more immune-specific antigen binding domains at the C-terminus of the dimerization domain.

In some embodiments, the polypeptide disclosed herein may comprise one or more immune-specific antigen binding domains at both the N- and C-terminus of the dimerization domain.

In exemplary and non-limiting embodiments, the polypeptide may comprise two immune-specific antigen binding domains at the C-terminus of the dimerization domain. In some embodiments, the immune-specific antigen binding domain that is immediately adjacent to the C-terminal part of the dimerization domain may be linked via a non-cleavable linker.

### Dimerization Domain (DD) and Protein Complex

The polypeptides of the present disclosure comprise a dimerization domain. As such, two polypeptides (polypeptide chains) may assemble to form a protein complex. Exemplary embodiments of protein complex include homodimers and heterodimers.

The dimerization domain may comprise, for example and without limitation, constant regions of an immunoglobulin, including for example a Fc, CH2 and/or CH3 domain of a heavy chain immunoglobulin.

In certain embodiments and aspects of the present disclosure the dimerization domain may have a sequence identical to that of a natural IgG1, IgG2, IgG3 or IgG4 constant region or with their corresponding CH2 and/or CH3 domains.

Particularly encompassed by the present disclosure dimerization domains having a sequence identical to that of a natural human antibody. Exemplary embodiments of dimerization domains include for example a CH2-CH3 domain of a natural human heavy chain.

When expressed in cells or in solution, polypeptides having a CH2-CH3 domain of a natural antibody have the propensity of forming dimers. When the two polypeptide chains of the protein complex are composed of the same amino acid sequence, the protein complex will form a homodimer. However, co-expression of polypeptides having a CH2-CH3 domain of a natural antibody, but different amino acid sequence will result in a mixture of homodimers and heterodimers. The different protein complexes present in a mixture may be separated by methods known in the art and including for example, size-exclusion chromatography.

Exemplary heterodimers of the present disclosure therefore include those having a CH2-CH3 domain of a natural antibody and that are formed by two polypeptides chains having different sequences or configurations.

However, the present disclosure also relates to polypeptides comprising a mutated dimerization domain.

Such polypeptides may thus comprise a mutated dimerization domain having, for example, one or more mutations in comparison with the sequence of a natural antibody. Exemplary embodiments of mutated dimerization domains include those having a natural CH2 domain and a mutated CH3 domain.

In some embodiments, the Fc region may be modified so as to prevent glycosylation, to extend its half-life, to modulate receptor binding or effector function. Exemplary mutations are discussed in Saunders K. O. (Front. Immunol. 10:1296, 2019 the entire content of which is incorporated herein by reference) and include for example mutation of asparagine 297 (e.g., N297).

Exemplary embodiments of dimerization domains are provided in SEQ ID NO:16 and SEQ ID NO:17. In both SEQ ID NO:16 and SEQ ID NO:17, amino acid residues 1-110 correspond to natural CH2 and amino acid residues 111-217 correspond to natural CH3. Amino acid residue No. 1 of SEQ ID NO:16 and SEQ ID NO:17 corresponds to position 231 in accordance with EU numbering system. Amino acid residue No. 111 of SEQ ID NO:16 and SEQ ID NO:17 corresponds to position to position 341 in accordance with EU numbering system.

Further exemplary embodiments of dimerization domains are provided SEQ ID NO:25 and SEQ ID NO:26. Additional exemplary embodiments of dimerization domains are provided in SEQ ID NO:48 and SEQ ID NO:50. Yet additional exemplary embodiments of dimerization domains are provided in SEQ ID NO: 47 and SEQ ID NO:49. Further exemplary embodiments of dimerization domain are provided in Table 5. Dimerization domains comprising the mutated Fc domains set forth in SEQ ID Nos: 53-91 are encompassed by the present disclosure. Dimerization domains comprising the mutated CH3 domains set forth in SEQ ID NO:92 to 95 are particularly contemplated.

More particularly, in some embodiments, polypeptides comprising the mutated CH3 domain set forth in SEQ ID NO:92 may form heterodimers with polypeptides comprising the mutated CH3 domain set forth in SEQ ID NO:93.

More particularly, in other embodiments, polypeptides comprising the mutated CH3 domain set forth in SEQ ID NO:94 may form heterodimers with polypeptides comprising the mutated CH3 domain set forth in SEQ ID NO:95.

In some embodiments, polypeptides comprising the mutated Fc domain set forth in SEQ ID NO:55 may form heterodimers with polypeptides comprising the mutated Fc domain set forth in SEQ ID NO:56.

In some embodiments, polypeptides comprising the mutated Fc domain set forth in SEQ ID NO:61 may form heterodimers with polypeptides comprising the mutated Fc domain set forth in SEQ ID NO:62.

In some embodiments, polypeptides comprising the mutated Fc domain set forth in SEQ ID NO:67 may form heterodimers with polypeptides comprising the mutated Fc domain set forth in SEQ ID NO:68.

In some embodiments, polypeptides comprising the mutated Fc domain set forth in SEQ ID NO:71 may form heterodimers with polypeptides comprising the mutated Fc domain set forth in SEQ ID NO:72.

In some embodiments, polypeptides comprising the mutated Fc domain set forth in SEQ ID NO:77 may form heterodimers with polypeptides comprising the mutated Fc domain set forth in SEQ ID NO:90.

In some embodiments, polypeptides comprising the mutated Fc domain set forth in SEQ ID NO:80 may form heterodimers with polypeptides comprising the mutated Fc domain set forth in SEQ ID NO:90.

In some embodiments, polypeptides comprising the mutated Fc domain set forth in SEQ ID NO:82 may form heterodimers with polypeptides comprising the mutated Fc domain set forth in SEQ ID NO:90.

In some embodiments, the polypeptides may have a mutated dimerization domain that comprises, for example, from 1 to 30, from 1 to 20, from 1 to 15, from 1 to 10, from 1 to 9, from 1 to 8, from 1 to 7, from 1 to 6, from 1 to 5, from 1 to 4, from 1 to 3 amino acid substitutions in comparison with a natural or wild type sequence.

In exemplary embodiments, mutated dimerization domains may comprise 1, 2, 3, 4, 5, 6, 7, 8, 9 or 10 amino acid substitutions. Amino acid substitutions may be conservatives or non-conservatives as outlined in Table A.

In exemplary embodiments, the polypeptides may have a mutated dimerization domain having a sequence which is from 80% to 99% identical with that of a natural IgG1, IgG2, IgG3 or IgG4 constant region or with a CH2 and/or CH3 domain. Polypeptides encompassed by the present disclosure include those comprising a mutated dimerization domain that is from 85% to 99% identical, from 90% to 99% identical, from 95% to 99% identical with that of a natural IgG1, IgG2, IgG3 or IgG4 constant region or with a CH2 and/or CH3 domain.

In some embodiments, the polypeptides of the present disclosure may comprise a mutated dimerization domain comprising amino acid substitutions that favorize heterodimer formation. Heterodimers of the present disclosure may therefore be formed by polypeptides comprising such mutations.

In some embodiments, the mutated dimerization domain may include amino acid substitutions at position 356, 357, 370, 399 and/or 439 (in accordance with EU numbering system).

In exemplary embodiments, one polypeptide chain of a given heterodimer may include a mutated CH3 having, for example, amino acid substitutions at positions 357, 399 and 439, whereas the other polypeptide chain of the heterodimer may include a mutated CH3 having, for example, amino acid substitutions at positions 356, 370 and 399 (in accordance with EU numbering system).

In exemplary embodiments, one polypeptide chain of a given heterodimer may include a mutated CH3 having, for example, amino acid substitutions at positions 357, 399 and 439, whereas the other polypeptide chain of the heterodimer may include a mutated CH3 having, for example, amino acid substitutions at positions 356, 370 and 399 (in accordance with EU numbering system). One or both polypeptide chains of a given heterodimer may optionally further comprise mutations at positions selected from 349, 350, 351, 352, 354, 355, 394 and/or 395. One polypeptide chain of a given heterodimer may thus comprise a first dimerization domain (DD₁) having the amino acid sequence disclosed herein and the other polypeptide chain of a given heterodimer may thus comprise a second dimerization domain (DD₂) having the amino acid sequence disclosed herein.

In particular aspects and embodiments, the polypeptide of the present disclosure may comprise in a N- to C-terminal fashion an amino acid sequence having the configuration set forth in formula Ic:

X-[(Abₐ)-(L_{b})]ₘ-(DD)-[(L_{c})-(Abₐ)]ₙ-Y

Wherein m may be 0, 1 or an integer greater than 1;
Wherein n may be 0, 1 or an integer greater than 1, provided that m and n are not 0 simultaneously;
Wherein Abₐ, Ab_{d}, may each independently comprise an antigen binding domain comprising one or more complementarity determining regions (CDRs) of an antibody;
Wherein X or Y may independently be present or absent and may comprise an amino acid sequence;
Wherein L_{b}, L_{c}, may each independently comprise one or more linkers; and
Wherein DD may comprise a dimerization domain comprising a) a CH3 domain comprising one or more mutations at positions corresponding to D399, D/E356 and/or K370 in accordance with EU numbering or b) a CH3 domain comprising one or more mutations at positions corresponding to D399, E357 and/or K439 in accordance with EU numbering.

In some embodiments, the amino acid at position 356 may be replaced by a neutral amino acid. In some embodiments, the amino acid at position 370 may be replaced by a positively charged amino acid. In some embodiments, the amino acid at position 399 may be replaced by a neutral amino acid. In some embodiments, the amino acid at position 357 may be replaced by a neutral amino acid. In some embodiments, the amino acid at position 439 may be replaced by a negatively charged amino acid.

For example, in order to favorize heterodimer formation, one of the polypeptide chain may be mutated by replacing a) the aspartic acid (D) or glutamic acid (E) at position 356 for a neutral amino acid, b) the lysine (K) at position 370 for a positively charged amino acid and c) the aspartic acid (D) at position 399 for a neutral amino acid while the other polypeptide chain may be mutated by replacing a) the glutamic acid (E) at position 357 for a neutral amino acid, b) the aspartic acid (D) at position 399 for a neutral amino acid and c) the lysine (K) at position 439 for a negatively charged amino acid.

An exemplary embodiment of the polypeptides of the present disclosure may comprise a mutated dimerization domain comprising a CH3 domain in which the aspartic acid (D) or glutamic acid (E) at position 356 may be changed for glutamine (Q), the lysine (K) at position 370 may be changed for glutamic acid (E) and the aspartic acid (D) at position 399 may be changed for asparagine (N).

Another exemplary embodiment of the polypeptides of the present disclosure may comprise a mutated dimerization domain comprising a CH3 domain in which the glutamic acid (E) at position 357 may be changed for glutamine (Q), the aspartic acid (D) at position 399 may be changed for asparagine (N) and the lysine (K) at position 439 may be changed for glutamic acid (E).

Heterodimers can be made by co-expressing a polypeptide chain (Chain A) comprising a CH3 domain in which the aspartic acid (D) or glutamic acid (E) at position 356 is changed for glutamine (Q), the lysine (K) at position 370 is changed for glutamic acid (E) and the aspartic acid (D) at position 399 is changed for asparagine (N) and a polypeptide chain (Chain B) comprising a CH3 domain in which the glutamic acid (E) at position 357 is changed for glutamine (Q), the aspartic acid (D) at position 399 is changed for asparagine (N) and the lysine (K) at position 439 is changed for glutamic acid (E).

Depending on the ratio of Chain A over Chain B, it is also possible to form homodimers upon co-expression of two such polypeptides.

Co-expression of a polypeptide Chain A with polypeptide Chain B may therefore result in heterodimers of Chain A and Chain B, homodimers of Chain A, homodimers of Chain B and mixture thereof. It is also possible that residual monomers of Chain A and/or Chain B exist. Since the monomers, heterodimers and homodimers each contain antigen binding domains, each component of the mixture may have some level of activity.

Therefore, monomers, heterodimers and homodimers that comprise the CH3 mutations disclosed herein as well as mixture of such monomers, heterodimers and/or homodimers are encompassed by the present disclosure.

In other embodiments, the polypeptide chains and the protein complexes disclosed herein may comprise a mutated dimerization domain that comprises mutations known in the art to favorize heterodimer formation.

For example, polypeptides and protein complex of the present disclosure may comprise the configuration set forth in formula Ia, formula Ib, formula II, formula III, formula IV, formula V, formula VI, formula VII or formula VIII and mutations known in the art to favorize heterodimer formation.

Exemplary embodiments of such mutations are disclosed for example in Ha, J-H et al. (Front Immunol, 2016; 7:394) or Godar M et al. (Expert Opinion on Therapeutic patents, 2018; 28(3):251-276), the entire content of which is incorporated by reference and includes for example Knobs-into-holes (first CH3 domain mutation T366Y and second CH3 domain mutation Y407T, first CH3 domain mutation T366W and second CH3 domain mutations T366S, L368A, Y407V, or first CH3 domain mutations S354C, T366W and second CH3 domain mutations Y349C, T366S, L368A, Y407V), DD/KK mutations (first CH3 domain mutations K409D, K392D, second CH3 domain mutations D399K, E356K), asymmetric re-engineering technology (first CH3 domain mutations E356K, E357K, D399K and second CH3 domain mutations K439E, K370E, K409D), BiMAb mutations (first CH3 domain mutations K249E, K288E, second CH3 domain mutations E236K, D278K), XmAb mutations (first CH3 domain mutations S364H, F405A, second CH3 domain mutations Y349T, T394F), DuoBody mutations (first CH3 domain mutation F405L, second CH3 domain mutation K409R), Azymetric mutations (first CH3 domain mutations T350V, L351Y, S400E, F405A, Y407V, second CH3 domain mutations T350V, T366L, N390R, K392M, T394W), Biclonics mutations (first CH3 domain mutation T366K (+L351K), second CH3 domain mutations L351D or E or D at Y349, L368 or Y349+R355), ZW1 mutations (first CH3 domain mutations T350V, L351Y, F405A, Y407V second CH3 domain mutations T350V, T366L, K392L, T394W), 7.8.60 mutations (first CH3 domain mutations K360D, D399M, Y407A, second CH3 domain mutations E345R, Q347R, T366V, K409V), EW-RVT mutations (first CH3 domain mutations K360E, K409W and second CH3 domain mutations Q347R, D399V, F405T), EW-RVTs-s mutations (first CH3 domain mutations K360E, K409W, Y349C and second CH3 domain mutations Q347R, D399V, F405T, S354C), SEED mutations (first CH3 domain mutations IgA-derived 45 residues on IgG1 CH3 and second CH3 domain mutations IgG1-derived 57 residues on IgA CH3), A107 mutations (first CH3 domain mutations K370E, K409W, second CH3 domain mutations E357N, D399V, F405T) etc.

The protein complex of the present disclosure may be formed by the assembly of two polypeptide chains having the same configuration (with same or different amino acid sequence) or having different configurations where the same or different configurations may be selected from the configuration set forth in formula Ia, formula Ib, formula Ic, formula II, formula III, formula IV, formula V, formula VI, formula VII and/or formula VIII.

In some embodiments, both polypeptide chains of a protein complex may have the configuration set forth in formula II (with same or different amino acid sequence).

In some embodiments, both polypeptide chains of a protein complex may have the configuration set forth in formula III (with same or different amino acid sequence).

In some embodiments, one of the polypeptide chains may have the configuration set forth in formula II, while the other may have the configuration set forth in formula III.

In some embodiments, one of the polypeptide chains may have the configuration set forth in formula II, while the other has the configuration set forth in formula IV.

In some embodiments, one of the polypeptide chains may have the configuration set forth in formula III, while the other has the configuration set forth in formula IV.

In some embodiments, one of the polypeptide chains may have the configuration set forth in formula IV, while the other has the configuration set forth in formula IV.

A protein complex composed of multivalent polypeptide chains is referred to herein as a multivalent protein complex.

A protein complex composed of two multispecific polypeptide chains is referred to herein as a multispecific protein complex.

The term “multispecific protein complex” encompasses “bispecific protein complex”, “tri specific protein complex”, “tetraspecific protein complex”, “pentaspecific protein complex”, “hexaspecific protein complex” and the like.

Exemplary embodiments of bispecific protein complexes include those having two polypeptides each chain comprising different tumor-specific antigen binding domains while the other antigen binding domains of the two polypeptides are identical or binds to the same antigen or epitope.

### Other Types of Dimers

The mutated dimerization domain disclosed herein may be used for dimerization of other types of polypeptide chains.

In some embodiments, the mutated dimerization domain or CH3 domain disclosed herein can be fused to binding domains or introduced within an antibody heavy chain or Fc region as to generate a bispecific IgG or IgG-like molecule. Exemplary embodiments of such bispecific molecule include bispecific antibodies, single chain Fv-CH3 (scFv-CH3) fusion, tandem-scFv-CH3 (TaFv-CH3) fusion, diabody-CH3 (db-CH3) fusion, tandem db-CH3 (TaDb-CH3) fusion, single chain db-CH3 fusion (scDb-CH3), Fab-CH3 fusion, single chain Fab-CH3 fusion, Fab-scFv-CH3 fusion, dual affinity retargeting (DART)-CH3 fusion, Fab-DART-CH3 fusion, single chain Fv-Fc (scFv-Fc) fusion, tandem-scFv-Fc (TaFv-Fc) fusion, diabody-Fc (db-Fc) fusion, tandem db-Fc (TaDb-Fc) fusion, single chain db-Fc fusion (scDb-Fc), Fab-Fc fusion, single chain Fab-Fc fusion, Fab-scFv-Fc fusion, dual affinity retargeting (DART)-Fc fusion, Fab-DART-Fc fusion etc.

In other embodiments, the mutated dimerization domain disclosed herein may be introduced into soluble decoy receptor traps.

The present disclosure thus relates to a protein complex comprising a) a first polypeptide chain comprising a Fc region, a CH3 or a CH2/CH3 domain comprising a substitution of the aspartic acid (D) or glutamic acid (E) at position 356 for a neutral amino acid, a substitution of the lysine (K) at position 370 for a positively charged amino acid and a substitution of the aspartic acid (D) at position 399 for a neutral amino acid and b) a second polypeptide chain comprising a Fc region, a CH3 or a CH2/CH3 domain comprising a substitution of the glutamic acid (E) at position 357 for a neutral amino acid, a substitution of the aspartic acid (D) at position 399 for a neutral amino acid and a substitution of the lysine (K) at position 439 for a negatively charged amino acid.

In some embodiments, the first polypeptide chain may comprise a CH3 domain in which the aspartic acid (D) or glutamic acid (E) at position 356 may be changed for glutamine (Q), the lysine (K) at position 370 may be changed for glutamic acid (E) and the aspartic acid (D) at position 399 may be changed for asparagine (N), and the second polypeptide chain may comprise a CH3 domain in which the glutamic acid (E) at position 357 may be changed for glutamine (Q), the aspartic acid (D) at position 399 may be changed for asparagine (N) and the lysine (K) at position 439 may be changed for glutamic acid (E).

In some embodiments, the first polypeptide chain and/or second polypeptide chain may comprise a CH3 domain comprising further mutations at positions corresponding to 349, 350, 351, 352, 354, 355, 394 and/or 395 in accordance with EU numbering.

In some embodiments, the first polypeptide chain may comprise a CH3 domain comprising mutations D399Q, D/E356Q, K370E, Y349K and S354K in accordance with EU numbering and the second polypeptide chain may comprise a CH3 domain comprising mutations D399Q, E357Q, K439E, Y349D and S354D in accordance with EU numbering.

In some embodiments, the first polypeptide chain may comprise a CH3 domain comprising mutations D399N, D/E356Q, K370E and L351W in accordance with EU numbering and the second polypeptide chain may comprise a CH3 domain comprising mutations D399N, E357Q, K439E and L351R in accordance with EU numbering.

In some embodiments, the first polypeptide chain may comprise a CH3 domain comprising mutations D399N, D/E356Q, K370E and S354M in accordance with EU numbering and the second polypeptide chain may comprise a CH3 domain comprising mutations D399N, E357Q, K439E and L351Y in accordance with EU numbering.

In some embodiments, the first polypeptide chain may comprise a CH3 domain comprising mutations D399N, D/E356Q, K370E and T350I in accordance with EU numbering and the second polypeptide chain may comprise a CH3 domain comprising mutations D399N, E357Q, K439E and T350I in accordance with EU numbering.

In some embodiments, the first polypeptide chain may comprise a CH3 domain comprising mutations D399N, D/E356Q, K370E and T350V in accordance with EU numbering and the second polypeptide chain may comprise a CH3 domain comprising mutations D399N, E357Q, K439E and T350V in accordance with EU numbering.

In some embodiments, the first polypeptide chain may comprise a CH3 domain comprising mutations D399N, D/E356Q, K370E and P352R in accordance with EU numbering and the second polypeptide chain may comprise a CH3 domain comprising mutations D399N, E357Q, K439E and L351R in accordance with EU numbering.

In some embodiments, the first polypeptide chain may comprise a CH3 domain comprising mutations D399N, D/E356Q, K370E and P352E in accordance with EU numbering and the second dimerization polypeptide chain may comprise a CH3 domain comprising mutations D399N, E357Q, K439E and L351R in accordance with EU numbering.

Combinations of first and second polypeptide chains respectively comprising Chain A and Chain B CH3 domains disclosed herein are also contemplated.

The first polypeptide and second polypeptide chain may be an antibody heavy chain.

The present disclosure thus particularly relates to an antibody or an antigen binding fragment thereof comprising a) a first heavy chain comprising a CH3 domain in which the aspartic acid (D) or glutamic acid (E) at position 356 may be changed for glutamine (Q), the lysine (K) at position 370 may be changed for glutamic acid (E) and the aspartic acid (D) at position 399 may be changed for asparagine (N), b) a second heavy chain comprising a CH3 domain in which the glutamic acid (E) at position 357 may be changed for glutamine (Q), the aspartic acid (D) at position 399 may be changed for asparagine (N) and the lysine (K) at position 439 may be changed for glutamic acid (E) and c) light chains.

In some embodiments the antibody or an antigen binding fragment thereof may comprise a) a first heavy chain comprising a CH3 domain in which the aspartic acid (D) or glutamic acid (E) at position 356 may be changed for glutamine (Q), the lysine (K) at position 370 may be changed for glutamic acid (E), the aspartic acid (D) at position 399 may be changed for glutamine (Q), the tyrosine (Y) at position 349 may be changed for lysine (K) and the serine (S) at position 354 may be changed for lysine (K), b) a second heavy chain comprising a CH3 domain in which the glutamic acid (E) at position 357 may be changed for glutamine (Q), the aspartic acid (D) at position 399 may be changed for glutamine (Q), the lysine (K) at position 439 may be changed for glutamic acid (E), the tyrosine (Y) at position 349 may be changed for aspartic acid (D) and the serine (S) at position 354 may be changed for aspartic acid (D) and c) light chains.

In some embodiments the antibody or an antigen binding fragment thereof may comprise a) a first heavy chain comprising a CH3 domain in which the aspartic acid (D) or glutamic acid (E) at position 356 may be changed for glutamine (Q), the lysine (K) at position 370 may be changed for glutamic acid (E), the aspartic acid (D) at position 399 may be changed for asparagine (N) and the leucine (L) at position 351 may be changed for tryptophan (W), b) a second heavy chain comprising a CH3 domain in which the glutamic acid (E) at position 357 may be changed for glutamine (Q), the aspartic acid (D) at position 399 may be changed for asparagine (N), the lysine (K) at position 439 may be changed for glutamic acid (E) and the leucine (L) at position 351 may be changed for arginine (R) and c) light chains.

In some embodiments the antibody or an antigen binding fragment thereof may comprise a) a first heavy chain comprising a CH3 domain in which the aspartic acid (D) or glutamic acid (E) at position 356 may be changed for glutamine (Q), the lysine (K) at position 370 may be changed for glutamic acid (E), the aspartic acid (D) at position 399 may be changed for asparagine (N) and the serine (S) at position 354 may be changed for methionine (M), b) a second heavy chain comprising a CH3 domain in which the glutamic acid (E) at position 357 may be changed for glutamine (Q), the aspartic acid (D) at position 399 may be changed for asparagine (N), the lysine (K) at position 439 may be changed for glutamic acid (E) and the leucine (L) at position 351 may be changed for tyrosine (Y) and c) light chains.

In some embodiments the antibody or an antigen binding fragment thereof may comprise a) a first heavy chain comprising a CH3 domain in which the aspartic acid (D) or glutamic acid (E) at position 356 may be changed for glutamine (Q), the lysine (K) at position 370 may be changed for glutamic acid (E), the aspartic acid (D) at position 399 may be changed for asparagine (N) and the threonine (T) at position 350 may be changed for isoleucine (I), b) a second heavy chain comprising a CH3 domain in which the glutamic acid (E) at position 357 may be changed for glutamine (Q), the aspartic acid (D) at position 399 may be changed for asparagine (N), the lysine (K) at position 439 may be changed for glutamic acid (E) and the threonine (T) at position 350 may be changed for isoleucine (I) and c) light chains.

In some embodiments the antibody or an antigen binding fragment thereof may comprise a) a first heavy chain comprising a CH3 domain in which the aspartic acid (D) or glutamic acid (E) at position 356 may be changed for glutamine (Q), the lysine (K) at position 370 may be changed for glutamic acid (E), the aspartic acid (D) at position 399 may be changed for asparagine (N) and the threonine (T) at position 350 may be changed for valine (V), b) a second heavy chain comprising a CH3 domain in which the glutamic acid (E) at position 357 may be changed for glutamine (Q), the aspartic acid (D) at position 399 may be changed for asparagine (N), the lysine (K) at position 439 may be changed for glutamic acid (E) and the threonine (T) at position 350 may be changed for valine (V) and c) light chains.

In some embodiments the antibody or an antigen binding fragment thereof may comprise a) a first heavy chain comprising a CH3 domain in which the aspartic acid (D) or glutamic acid (E) at position 356 may be changed for glutamine (Q), the lysine (K) at position 370 may be changed for glutamic acid (E), the aspartic acid (D) at position 399 may be changed for asparagine (N) and the proline (P) at position 352 may be changed for arginine (R), b) a second heavy chain comprising a CH3 domain in which the glutamic acid (E) at position 357 may be changed for glutamine (Q), the aspartic acid (D) at position 399 may be changed for asparagine (N), the lysine (K) at position 439 may be changed for glutamic acid (E) and the leucine (L) at position 351 may be changed for arginine (R) and c) light chains.

In some embodiments the antibody or an antigen binding fragment thereof may comprise a) a first heavy chain comprising a CH3 domain in which the aspartic acid (D) or glutamic acid (E) at position 356 may be changed for glutamine (Q), the lysine (K) at position 370 may be changed for glutamic acid (E), the aspartic acid (D) at position 399 may be changed for asparagine (N) and the proline (P) at position 352 may be changed for glutamic acid (E), b) a second heavy chain comprising a CH3 domain in which the glutamic acid (E) at position 357 may be changed for glutamine (Q), the aspartic acid (D) at position 399 may be changed for asparagine (N), the lysine (K) at position 439 may be changed for glutamic acid (E) and the leucine (L) at position 351 may be changed for arginine (R) and c) light chains.

Such antibody or antigen binding fragment thereof include bi-specific antibodies or bi-specific antigen binding fragments thereof.

### Linkers (L)

The different modules of the polypeptide chains disclosed herein may be associated to each other via linkers.

In some embodiments, the linkers used to join one or more modules of the polypeptide chain are not cleavable linkers.

In an exemplary embodiment, the linker located immediately adjacent to the C-terminal end of the dimerization domain (Lc) does not comprise a cleavable linker.

In another exemplary embodiment, at least one of the linkers located between two antigen binding domains do not comprise a cleavable linker.

In other embodiments the linkers used to join one or more modules of the polypeptide chain may include non-cleavable linkers.

In an exemplary embodiment, the linker located immediately adjacent to the C-terminal end of the dimerization domain is a non-cleavable linker.

In another exemplary embodiment, at least one of the linkers located between two antigen binding domains is a non-cleavable linker.

In a further exemplary embodiment, the linker located immediately adjacent to the C-terminal end of the dimerization domain and the linker joining the first two antigen binding domains located at the C-terminal end of the dimerization domain are non-cleavable linkers.

In some embodiment, the linker immediately adjacent to the N-terminal end of the dimerization domain may preferably comprise hinge region of an antibody.

In some embodiments, all modules of the polypeptide chain are linked via non-cleavable linkers.

Exemplary embodiments of non-cleavable linkers include those that remains substantially intact during protein expression or during manufacturing process. As used herein “substantially intact” means that linker cleavage occurs in 20% or less, in 15% or less, in 10% or less, in 7.5% or less, in 5% or less, in 4% or less, in 3% or less, in 2% or less, in 1% or less of the total polypeptide content of a given solution or composition.

Other exemplary embodiments of non-cleavable linkers also include linkers that do not comprise a specific cleavage site for one or more proteases present in human or animal blood or serum.

Additional exemplary embodiments of non-cleavable linkers further include linkers that retain their integrity for at least one, two, three, four, five, six, twelve, twenty-four, forty-eight hours or more after administration upon administration of the polypeptide or protein complex in individuals.

In further exemplary embodiments, a linker comprises both non-cleavable linkers and cleavable linkers.

In some embodiments, a linker is not cleavable.

In some instance cleavable linkers may be used for in vivo release of drugs (e.g., cytostatic molecules, cytotoxic molecules, chemotherapeutics etc.) or labels attached to the polypeptide of the present disclosure.

Exemplary embodiments of cleavable linkers are provided for example in US2019/0010242 and include linkers that are sensitive to cleavage by a protease, usually an extracellular protease, such as a protease that is produced by a tumor or an activated immune effector cell and include those having a site for specific cleavage by proteases selected from ADAMS, ADAMTS, e.g. ADAMS; ADAMS; ADAM10; ADAM12; ADAM15; ADAM17/TACE; ADAMDEC1; ADAMTS1; ADAMTS4; ADAMTS5; aspartate proteases, e.g., BACE or Renin; aspartic cathepsins, e.g., Cathepsin D or Cathepsin E; Caspases, e.g., Caspase 1, Caspase 2, Caspase 3, Caspase 4, Caspase 5, Caspase 6, Caspase 7, Caspase 8, Caspase 9, Caspase 10, or Caspase 14; cysteine cathepsins, e.g., Cathepsin B, Cathepsin C, Cathepsin K, Cathepsin L, Cathepsin S, Cathepsin V/L2, Cathepsin X/Z/P; cysteine proteinases, e.g., Cruzipain; Legumain; Otubain-2; KLKs, e.g., KLK4, KLKS, KLK6, KLK7, KLK8, KLK10, KLK11, KLK13, or KLK14; metalloproteinases, e.g., Meprin; Neprilysin; PSMA; BMP-1; MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, or MMP27, serine proteases, e.g., activated protein C, Cathepsin A, Cathepsin G, Chymase, coagulation factor proteases (e.g., FVIIa, FIXa, FXa, FXIa, FXIIa), Elastase, granzyme B, Guanidinobenzoatase, HtrAl, Human Neutrophil Elastase, Lactoferrin, Marapsin, NS3/4A, PACE4, Plasmin, PSA, tPA, Thrombin, Tryptase, uPA; Type II Transmembrane Serine Proteases (TTSPs), e.g., DESC1, DPP-4, FAP, Hepsin, Matriptase-2, Matriptase, TMPRSS2, TMPRSS3, or TMPRSS4; and any combination thereof. In some embodiments the polypeptides of the present disclosure do not include such linkers at position corresponding to Lc.

Exemplary embodiments of linkers include flexible linkers, rigid linkers, helical linkers and combination thereof. Linkers are discussed for example, in Chen X et al. (Adv Drug Deliv Rev. 2013; 65(10):1357-1369) the entire content of which is incorporated herein by reference.

In some embodiments, an antibody hinge region or a portion thereof may be used to link a module to the dimerization domain and is considered herein as a linker. The hinge region may be derived from a natural antibody (of human or animal origin) or from a synthetic antibody. Hinge regions may be obtained, for example, from IgGs such as IgG1, IgG2, IgG3 or IgG4. Exemplary embodiments of hinge regions are provided in SEQ ID NO:1, SEQ ID NO:35, SEQ ID NO:39 and SEQ ID NO:43.

In some instances, the hinge region may have a sequence that is from 80% to 99% identical with that of a natural IgG1, IgG2, IgG3 or IgG4 hinge region. An exemplary and non-limiting embodiment of a mutated hinge includes a hinge region of an IgG4 in which S228 is replaced with P (EU numbering) (Angal, S. et al., Mol Immunol 30, 105-108, 1993). Other exemplary embodiments of mutated hinge are provided in SEQ ID NOs:32-34, 36-38, 40-42 and 44-46

Flexible linkers are usually composed of small polar amino acids such as threonine or serine and glycine. Exemplary and non-limiting embodiments of flexible linkers include GS linkers (glycine/serine repeats) such as for example, (GGGS)ₙ(GGGGS)ₘ, (GS), (G₄S)ₙ, (GGS)ₙ, (GGGS)ₙ, (GGGGS)ₙ, (GGSG)ₙ, (GGGSS)ₙ wherein n and m may be an integer such as 1, 2, 3, 4, 5, 6, 7, 8, 9, 10 or more, such as 15, 20 or 25.

Specific exemplary and non-limiting embodiments of flexible linkers include those comprising of consisting of the amino acid sequence set forth in SEQ ID NO: 3, SEQ ID NO:4, SEQ ID NO:5, SEQ ID NO:6 or SEQ ID NO:7.

It is to be understood that SEQ ID NO:7 may be represented by formula (GGGGS)ₙ wherein n is an integer selected from 1 to 10 or alternatively by formula GGGGSX₁ wherein X₁ is absent or, if present, is from 1 to 9 repeats of amino acid residues 1 to 5 of SEQ ID NO:7.

Rigid linkers of the present disclosure are usually composed of proline-rich sequences (XP)ₙ, wherein X designate any amino acid, preferably Ala, Lys or Glu and n is an integer such as 1, 2, 3, 4, 5, 6, 7, 8, 9, 10 etc. (Chen X et al., 2013).

Specific exemplary and non-limiting embodiments of rigid linkers include those comprising of consisting of the amino acid sequence set forth in SEQ ID NO:8, SEQ ID NO:9, SEQ ID NO:10 or SEQ ID NO:11.

It is to be understood that SEQ ID NO:11 may be represented by formula (X(PAPAP))ₙKA wherein n is an integer selected from 1 to 10, wherein X is present or absent and, if present, is A or, alternatively, SEQ ID NO: 11 may be represented by formula (XPAPAP)X₂KA wherein X may be present or absent and, if present, is A; and wherein X2 is absent or, if present, is from 1 to 9 repeats of amino acid residues 1 to 6 of SEQ ID NO:11.

Helical linkers may sometimes be characterized as rigid but are herein being separated into a distinct linker family. Exemplary embodiments of helical linkers are discussed in Chen X et al., 2013 and comprise, for example, repeats of alanine residues flanked by a positively charged- and a negatively charged amino acid residue.

Specific exemplary and non-limiting embodiments of helical linkers include those comprising of consisting of the amino acid sequence set forth in SEQ ID NO:12, SEQ ID NO:13, SEQ ID NO:14 and SEQ ID NO:15.

It is to be understood that SEQ ID NO:15 may be represented by formula X(EAAAK)ₙX₂ wherein n is an integer selected from 1 to 10, more preferably 2-5 wherein X and X2 are independently present or absent and, if present, is preferably A. Alternatively, SEQ ID NO: 15 may be represented by formula X(EAAAK)X₃X₂, wherein X and X2 are independently present or absent and, if present, is preferably A; and X3 is absent or, if present, is from 1 to 9 repeats of amino acid residues 2 to 6 of SEQ ID NO:15.

In an exemplary embodiment the linker immediately adjacent to the C-terminal end of the dimerization domain (identified as Linker 2 in Tables 1-4 or as L_{c1} in formulas II to VIII) may comprise either a flexible linker, a rigid linker or a helical linker. Linkers that may be particularly selected to occupy this position include for example and without limitations, a linker comprising or consisting of the amino acid sequence set forth in SEQ ID NO: 3-12, SEQ ID NO:14 or in SEQ ID NO:15 wherein n is 1.

In an exemplary embodiment the linker joining the first two antigen binding domains located at the C-terminal end of the dimerization domain (identified as Linker 3 in Tables 1-4 or as L_{c2} in formulas II to VIII) may comprise either a flexible linker, a rigid linker or a helical linker. Linkers that may be particularly selected to occupy this position include for example and without limitations, a linker comprising or consisting of the amino acid sequence set forth in SEQ ID NO: 3-13, or in SEQ ID NO:15 wherein n is 1.

The present disclosure also provides linkers having an addition of from 1 to 10 amino acids (and any range or value comprised within 1 and 10 such as for example, from 1 to 5) at one or both the N- or C-terminus of any of SEQ ID NOs: 3 to 15. These additional amino acid residues may each independently be selected from any amino acid residues. These additional amino acid residues preferably form a non-cleavable sequence.

The present disclosure also provides linkers having a deletion of from 1, 2, 3, 4 or 5 amino acids (and any value comprised within 1 and 5) at one or both the N- or C-terminus of any of SEQ ID NOs: 3 to 15.

Suitable linkers may comprise, for example, an amino acid sequence comprising from about 3 to about 50, from about 3 to about 40, from about 3 to about 30, from about 3 to about 25, from about 3 to about 20, from about 3 to about 15, from about 3 to about 10 amino acid residues.

In exemplary embodiments, the length of each linker may independently range from about 5 to about 50 amino acid residues, including for example, from about 5 to about 40 amino acid residues, from about 10 to about 40 amino acid residues, from about 20 to about 40 amino acid residues, from about 20 to about 35 amino acid residues, from about 25 to about 30 amino acid residues and any sub-range comprised and including such ranges.

In some embodiments linkers that comprise the amino acid sequence set forth in SEQ ID NO:7, SEQ ID NO:11 or SEQ ID NO:15 may have a “n” value preferably from 1 to 10, more preferably from 2-5 including 2, 3, 4 or 5.

### Variants

Variants of the sequences disclosed herein are also encompassed by the present disclosure.

Variants encompassed by the present disclosure include those which may comprise an insertion of one or more amino acid residues at one or more position, a deletion of one or more amino acid residues at one or more position or a substitution of one or more amino acid residues at one or more position (conservative or non-conservative substitutions).

For example, naturally occurring residues are divided into groups based on common side chain properties. Conservative substitutions may be made by exchanging an amino acid from one of the groups listed below (group 1 to 6) for another amino acid of the same group. Non-conservative substitutions will entail exchanging a member of one of these groups for another.
(group 1) hydrophobic: norleucine, methionine (Met), Alanine (Ala), Valine (Val), Leucine (Leu), Isoleucine (Ile)
(group 2) neutral hydrophilic: Cysteine (Cys), Serine (Ser), Threonine (Thr), Asparagine (Asn), Glutamine (Gln),
(group 3) acidic: Aspartic acid (Asp), Glutamic acid (Glu)
(group 4) basic: Histidine (His), Lysine (Lys), Arginine (Arg)
(group 5) residues that influence chain orientation: Glycine (Gly), Proline (Pro); and
(group 6) aromatic: Tryptophan (Trp), Tyrosine (Tyr), Phenylalanine (Phe)

Other exemplary embodiments of conservative substitutions are shown in Table A under the heading of “preferred substitutions”. If such substitutions result in an undesired property, then more substantial changes, denominated “exemplary substitutions” in Table A, or as further described below in reference to amino acid classes, may be introduced and the products screened.

One of skill in the art will recognize that certain amino acids are less positively charged, are neutral, are negatively charged or have a reduced charge in comparison to other amino acids. Amino acids can be categorized based on net charge as indicated by an amino acid's isoelectric point. The isoelectric point is the pH at which the average net charge of the amino acid molecule is zero. When pH>pI, an amino acid has a net negative charge, and when the pH<pI, an amino acid has a net positive charge. In some embodiments, the measured pI value for an antibody is between about 3 and 9 (e.g. 3.0, 3.5, 4.0, 4.5, 5.0, 5.5, 6.0, 6.5, 7.0, 7.5, 8.0, 8.1, 8.2, 8.3, 8.4, 8.5, and 9) and any values in between. In some embodiments, the measured pI value for an antibody is between about 4 and 7 (e.g. 4.0, 4.5, 5.0, 5.5, 6.0, 6.5, 7.0), and any values in between. Exemplary isoelectric points of amino acids are shown in Table A below. Generally amino acids with positive electrically charged side chains include, for example, Arginine (R), Histidine (H), and Lysine (K). Amino acids with negative electrically charged side chains include, for example, Aspartic Acid (D) and Glutamic Acid (E). Amino acids with polar properties include, for example, Serine (S), Threonine (T), Asparagine (N), Glutamine (Q), and Cysteine (C), Tyrosine (Y) and Tryptophan (W). Non-polar amino acids include, for example, Alanine (A), Valine (V), Isoleucine (I), Leucine (L), Methionine (M), Phenylalanine (F), Glycine (G) and Proline (P).

In some embodiments, the isoelectric point of an antibody is modified via amino acid substitution. See, e.g. US20110076275. In some embodiments, modifying the isoelectric point of a polypeptide comprising an antibody results in a change in the antibody's half-life.

TABLE A
Exemplary amino acid substitutions

| Original | Exemplary | Conservative | pI |
|---|---|---|---|
| residue | substitution | substitution | (isoelectric point) |
| Ala (A) | Val, Leu, Ile | Val | 6.0 |
| Arg (R) | Lys, Gln, Asn | Lys | 10.76 |
| Asn (N) | Gln, His, Lys, Arg, Asp | Gln | 5.41 |
| Asp (D) | Glu, Asn | Glu | 2.77 |
| Cys (C) | Ser, Ala | Ser | 5.07 |
| Gln (Q) | Asn; Glu | Asn | 5.65 |
| Glu (E) | Asp, Gln | Asp | 3.22 |
| Gly (G) | Ala | Ala | 5.97 |
| His (H) | Asn, Gln, Lys, Arg, | Arg | 7.59 |
| Ile (I) | Leu, Val, Met, Ala, Phe, | Leu | 6.02 |
| | norleucine | | |
| Leu (L) | Norleucine, Ile, Val, | Ile | 5.98 |
| | Met, Ala, Phe | | |
| Lys (K) | Arg, Gln, Asn | Arg | 9.74 |
| Met (M) | Leu, Phe, Ile | Leu | 5.74 |
| Phe (F) | Leu, Val, Ile, Ala, Tyr | Tyr | 5.48 |
| Pro (P) | Ala | Ala | 6.30 |
| Ser (S) | Thr | Thr | 5.58 |
| Thr (T) | Ser | Ser | 5.60 |
| Trp (W) | Tyr, Phe | Tyr | 5.89 |
| Tyr (Y) | Trp, Phe, Thr, Ser | Phe | 5.66 |
| Val (V) | Ile, Leu, Met, Phe, Ala, | Leu | 5.96 |
| | norleucine | | |

Generally, the degree of similarity and identity between variable chains is determined herein using the Blast2 sequence program (Tatiana A. Tatusova, Thomas L. Madden (1999), “Blast 2 sequences—a new tool for comparing protein and nucleotide sequences”, FEMS Microbiol Lett. 174:247-250) using default settings, i.e., blastp program, BLOSUM62 matrix (open gap 11 and extension gap penalty 1; gapx dropoff 50, expect 10.0, word size 3) and activated filters.

Percent identity will therefore be indicative of amino acids which are identical in comparison with the original peptide and which may occupy the same or similar position.

Percent similarity will be indicative of amino acids which are identical and those which are replaced with conservative amino acid substitution in comparison with the original peptide at the same or similar position.

Variants of the present disclosure may therefore comprise a sequence that is at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical with that of an original or reference sequence or a portion of an original sequence.

In some embodiments, a variant may have at least 80% sequence identity with a sequence disclosed herein. In other embodiments, a variant may have at least 85% sequence identity with a sequence disclosed herein. In yet embodiments, a variant may have at least 90% sequence identity with a sequence disclosed herein. In further embodiments, a variant may have at least 95% sequence identity with a sequence disclosed herein. In other embodiments, a variant may have at least 99% sequence identity with a sequence disclosed herein.

Exemplary embodiments of variants include polypeptides or protein complexes that comprise a hinge, Fc, CH3, CH2/CH3 region that is derived from a natural antibody but that comprise one, two, three, four, five, six, seven, eight, nine, ten or more amino acid difference.

In some embodiments, the polypeptide of the present disclosure may thus comprise a hinge region that is at least 80% identical, at least 85% identical, at least 90% identical, at least 95% identical, at least 99% identical to a hinge region of a natural antibody.

In some embodiments, the polypeptide of the present disclosure may thus comprise a Fc portion that is at least 80% identical to a Fc of a natural antibody.

In some embodiments, the polypeptide of the present disclosure may thus comprise a CH2 domain that is at least 80% identical, at least 85% identical, at least 90% identical, at least 95% identical, at least 99% identical to the CH2 domain of a natural antibody.

In some embodiments, the polypeptide of the present disclosure may thus comprise a CH3 domain that is at least 80% identical, at least 85% identical, at least 90% identical, at least 95% identical, at least 99% identical to the CH3 domain of a natural antibody.

In some embodiments, the polypeptide of the present disclosure may thus comprise a CH2/CH3 domain that is at least 80% identical, at least 85% identical, at least 90% identical, at least 95% identical, at least 99% identical to the CH2/CH3 domain of a natural antibody.

### Nucleic Acids, Vectors, Kits, Cells and Method of Making Polypeptides

Nucleic acid molecules of the present disclosure may be single-stranded or double-stranded. The nucleic acid molecules disclosed herein may comprises deoxyribonucleotides, ribonucleotides, modified deoxyribonucleotides or modified ribonucleotides. The nucleic acid molecules of the present disclosure may comprise for example DNA.

DNA segments and vectors encoding one or more modules or entire polypeptide chains are particularly provided.

The DNA segments and/or vectors may be provided in separate vials and sold as a kit.

Particularly contemplated are sets of DNA segments that comprise sequence allowing directional assembly of the modules and cloning vectors that incorporate the DNA segments or entire polypeptide chains.

The DNA segments and vectors may be provided as part of a kit for assembling DNA constructs capable of expressing the polypeptides or protein complexes disclosed herein.

The kit may at least comprise one or more DNA segment or vectors that allow a user to generate a polypeptide chain comprising the mutated dimerization domain having amino acid substitutions at position 356, 357, 370, 399 and/or 439 (in accordance with EU numbering system) as disclosed herein.

Due to the inherent degeneracy of the genetic code, DNA sequences that encode the same, substantially the same or a functionally equivalent amino acid sequence may be produced and used. The nucleotide sequences of the present disclosure may be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth. Codon-optimized nucleic acids encoding the polypeptide chains described herein are encompassed by the present disclosure.

The polypeptides and protein complexes disclosed herein may be made by a variety of methods familiar to those skilled in the art, including by recombinant DNA methods or by in vitro transcription/translation.

Generally, the polypeptide chains described herein are expressed from nucleic acid sequences inserted into an expression vector, i.e., a vector that contains the elements for transcriptional and translational control of the inserted coding sequence in a particular host. These elements may include regulatory sequences, such as enhancers, constitutive and inducible promoters, and 5′ and 3′ un-translated regions.

A variety of expression vector/host cell systems known to those of skill in the art may be used to express the polypeptide chains described herein. In the event, that the protein complex is composed of distinct polypeptide chains, each of such polypeptide chain may be provided by separate expression vectors or by a unique expression vector. In accordance with the present disclosure, the two chains of a protein complex may be encoded by a single vector or by separate vectors (vector set).

Polypeptides are often expressed in mammalian cells. For long-term production of recombinant proteins, a stable expression system may be used in which the DNA segment is incorporated into the host cell genome or maintained in an episomal form by the use of selectable markers. A host cell type may be chosen for its ability to modulate expression of the inserted sequences or to process the expressed polypeptide in the desired fashion. Different host cells that have specific cellular machinery and characteristic mechanisms for post-translational activities (e.g., CHO, HeLa, MDCK, HEK293, and W138) are available commercially and from the American Type Culture Collection (ATCC) and may be chosen to ensure the correct modification and processing of the expressed polypeptide.

Other types of expression system can be used. These include, for example, microorganisms such as bacteria transformed with recombinant bacteriophage, plasmid, or cosmid DNA expression vectors; yeast transformed with yeast expression vectors; insect cell systems infected with baculovirus vectors; plant cell systems transformed with viral or bacterial expression vectors; or animal cell systems.

The present disclosure therefore relates to isolated cells transformed or transfected with a vector, nucleic acid, sets of vectors or sets of nucleic acids encoding at least one of the polypeptide chains described herein. The present disclosure therefore also relates to isolated cells capable or expressing the polypeptides or protein complex disclosed herein.

The present disclosure also relates to a method of making protein complexes. The method may comprise providing a cell (e.g., a mammalian cell) with a vector or sets of vectors encoding one or more of the polypeptide chains disclosed herein and allowing expression.

In some embodiments, the titer of the polypeptide and/or the protein complex produced by cells may be 0.1 g/L or more. In some instances, the titer of the polypeptide and/or the protein complex produced by cells may be 0.5 g/L or more. In some instances, the titer of the polypeptide and/or the protein complex produced by cells may be 1 g/L or more. In some instances, the titer of the polypeptide and/or the protein complex produced by cells may be 2 g/L or more. In some instances, the titer of the polypeptide and/or the protein complex produced by cells may be 3 g/L or more. In some instances, the titer of the polypeptide and/or the protein complex produced by cells may be 4 g/L or more. Usually, homodimers are made by transfection of cells with a vector comprising a nucleic acid sequence encoding one of the polypeptide chains disclosed herein. The collected supernatant may contain homodimers or a mixture of monomers and/or homodimers.

Generally, heterodimers are made by co-transfection of cells with at least two types of vectors (a vector set) each comprising a nucleic acid sequence encoding two distinct polypeptide chains. The proper ratio of Chain A over Chain B is generally dependent on the level of protein expression obtained from each individual plasmid and may vary for example from about 1:10 to about 10:1. A DNA ratio of approximately 1:1 is particularly preferred for some of the constructs disclosed herein.

Heterodimers can also be made by transfecting cells with a single vector encoding both polypeptide chains. The collected supernatant may contain heterodimers or a mixture of monomers, heterodimers and/or homodimers.

The method of making polypeptides of the present disclosure may further comprise a step of separating or isolating monomers, homodimers and heterodimers from a mixture that comprises. Homodimers or heterodimers may be purified and isolated, for example, by size exclusion chromatography or with the help of tags or by other methods known to a person of skill in the art.

The method may also comprise a step of isolating and/or purifying the protein complex from impurities.

The method of the present disclosure will therefore result in compositions comprising homodimers, heterodimers or a mixture of monomers heterodimers and/or homodimers.

In some exemplary embodiments, the composition may mainly comprise homodimers. In an exemplary embodiment, the composition may comprise a proportion of at least about 80%, at least 85%, at least 90%, at least 99% or 100% of homodimers.

In other exemplary embodiments, the composition may mainly comprise heterodimers. In an exemplary embodiment, the composition may comprise a proportion of at least about 80%, at least 85%, at least 90%, at least 99% or 100% of heterodimers.

### Conjugates

The polypeptides, polypeptide chain or protein complex of the present disclosure may be conjugated, for example, with a therapeutic moiety (for therapeutic purposes) or with a detectable moiety (i.e., for detection or diagnostic purposes) or to a protein allowing an extended half-life or is attached to nanoparticle. In some instances, therapeutic or detectable moieties may be linked to at least one amino acid residues of the polypeptide.

In an exemplary embodiment, the polypeptide, polypeptide chain or protein complex of the present disclosure is conjugated with a therapeutic moiety such as for example and without limitation, a chemotherapeutic, a cytokine, a cytotoxic agent, an anti-cancer drug (e.g., small molecule), and the like.

Therapeutic moiety may include, for example and without limitation, Yttrium-90, Scandium-47, Rhenium-186, Iodine-131, Iodine-125, and many others recognized by those skilled in the art (e.g., lutetium (e.g., Lu¹⁷⁷), bismuth (e.g., Bi²¹³), copper (e.g., Cu⁶⁷)), 5-fluorouracil, adriamycin, irinotecan, taxanes, pseudomonas endotoxin, ricin, auristatins (e.g., monomethyl auristatin E, monomethyl auristatin F), maytansinoids (e.g., mertansine) and other toxins.

In another exemplary embodiment, the polypeptide or protein complex of the present disclosure is conjugated with a detectable moiety including for example and without limitation, a moiety detectable by spectroscopic, photochemical, biochemical, immunochemical, chemical and/or other physical means. A detectable moiety may be coupled either directly and/or indirectly (for example via a linkage, such as, without limitation, a DOTA or NHS linkage) to the polypeptide or protein complex using methods well known in the art. A wide variety of detectable moieties may be used, with the choice depending on the sensitivity required, ease of conjugation, stability requirements and available instrumentation. A suitable detectable moiety include, but is not limited to, a fluorescent label, a radioactive label (for example, without limitation, 125I, In¹¹¹, Tc⁹⁹, I¹³¹ and including positron emitting isotopes for PET scanner etc), a nuclear magnetic resonance active label, a luminescent label, a chemiluminescent label, a chromophore label, an enzyme label (for example and without limitation horseradish peroxidase, alkaline phosphatase, etc.), quantum dots and/or a nanoparticle. Detectable moiety may cause and/or produce a detectable signal thereby allowing for a signal from the detectable moiety to be detected.

### Pharmaceutical Compositions

Pharmaceutical compositions comprising the polypeptides or protein complex of the present disclosure are also encompassed by the present disclosure. The pharmaceutical composition may also comprise a pharmaceutically acceptable carrier.

In some embodiments, the pharmaceutical composition comprises conjugated polypeptides or conjugated protein complex as disclosed herein. In some embodiments, the pharmaceutical composition comprises polypeptides or protein complex conjugated with a therapeutic moiety. In some embodiments, the pharmaceutical composition comprises polypeptides or protein complex is conjugated with a detectable label.

In addition to the active ingredients, a pharmaceutical composition may contain pharmaceutically acceptable carriers comprising water, PBS, salt solutions, gelatins, oils, alcohols, and other excipients and auxiliaries that facilitate processing of the active compounds into preparations that may be used pharmaceutically. In other instances, such preparations may be sterilized.

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also encompassed by the disclosure are particulate compositions coated with polymers (e.g., poloxamers or poloxamines). Other embodiments of the compositions of the disclosure incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” or “pharmaceutical carrier” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the pharmaceutical compositions described above may be applied to any subject in need of therapy, including, but not limited to, mammals such as dogs, cats, cows, horses, rabbits, monkeys, and especially humans.

The pharmaceutical compositions described herein may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

### Method of Use

The polypeptides, polypeptide chains and protein complexes of the present disclosure may be used for treatment of disorders or diseases.

In some embodiments, the polypeptides and protein complexes may be used to target therapeutics and/or diagnostics to a target cell, circulating protein or tissue.

In some embodiments, the polypeptides and protein complexes may be conjugated with a therapeutic moiety and used for therapeutic methods.

In some embodiments, the polypeptides and protein complexes may be conjugated with a detectable moiety and used for detection or diagnostic methods.

In some embodiments, the polypeptides, polypeptide chains and protein complexes of the present disclosure may be used for targeting tumors in vivo.

In some embodiments, the polypeptides, polypeptide chains and protein complexes are used for promoting tumor regression and/or reducing tumor volume in vivo.

The polypeptides, polypeptide chains and protein complexes of the present disclosure may thus be used for cancer treatment.

The method of the present disclosure may comprise a step of administering the polypeptides, protein complexes or mixture disclosed herein or a pharmaceutical composition comprising the polypeptides, protein complexes or mixture to an individual in need.

In some embodiments, the polypeptides, polypeptide chains and protein complexes are administered in combination with a chemotherapeutic.

In accordance with the present disclosure, the individual in need may be a human. Further in accordance with the present disclosure, the individual in need may be an animal.

In some embodiment, treatment of disorders or diseases that are caused or associated with expression of a neo-antigen are particularly contemplated.

In some embodiment, treatment of disorders or diseases that are caused or associated with expression over expression of an antigen are particularly contemplated.

In some embodiments, the disorder or disease may be cancer.

In other embodiments, the disorder or disease may be an infection.

In other embodiments, the disorder or disease may be an immune dysregulation.

In other embodiments, the disorder or disease may be a metabolic dysregulation.

The polypeptides and protein complexes of the present disclosure may be used for detection purposes.

Detection of a particular target may be performed in vitro by contacting a sample, containing or suspected of containing the target with a polypeptide or protein complex comprising an antigen binding domain for such target and quantifying a signal associated with positive or negative binding using a detection apparatus.

The sample may originate from a mammal (e.g., a human). The sample may be a tissue sample obtained from the mammal or a cell culture supernatant.

In some embodiments, the sample may be a serum sample, a plasma sample, a blood sample, semen or ascitic fluid obtained from the mammal.

Detection of a particular target may be performed in vivo by administering a polypeptide or protein complex comprising an antigen binding domain for such target to an individual and quantifying a signal associated with positive or negative binding using a detection apparatus.

Upon detecting the presence of the target in the sample or in the individual, a drug (e.g., antibody, small molecule, a polypeptide or protein complex disclosed herein) may be administered to the individual.

In addition to the embodiments described and provided in this disclosure, the following non-limiting embodiments are particularly contemplated.

### EXAMPLES

### Example 1. Methods of Producing Polypeptides

### Selection of VHHs

Heavy chain only antibodies are produced for example, by immunization of camelids or transgenic animals or from synthetic libraries of such antibodies. The sequences of the antigen binding domains are selected and expressed, for example, as VHH-hinge-Fc fusions or by phage display and tested for their biological activity in vitro and/or in vivo. Antigen binding domains are assembled into a single polypeptide chains based on the different formats outlined in the present disclosure and polypeptide chains or protein complexes, including homodimers and heterodimers are produced in cells and tested for their overall biological activity in vitro and/or in vivo or for the biological activity of the different modules.

### Gene Synthesis and Assembly of Constructs

Segments of DNA corresponding to genes or gene fragments (DNA modules) were synthesized using the GeneArt® system (Thermo Fisher Scientific). The DNA modules were designed with recognition sites for the type II S restriction enzyme BsaI that when digested with BsaI generate unique overhangs. The sequence of these overhangs directs the position of the modules in the DNA construct.

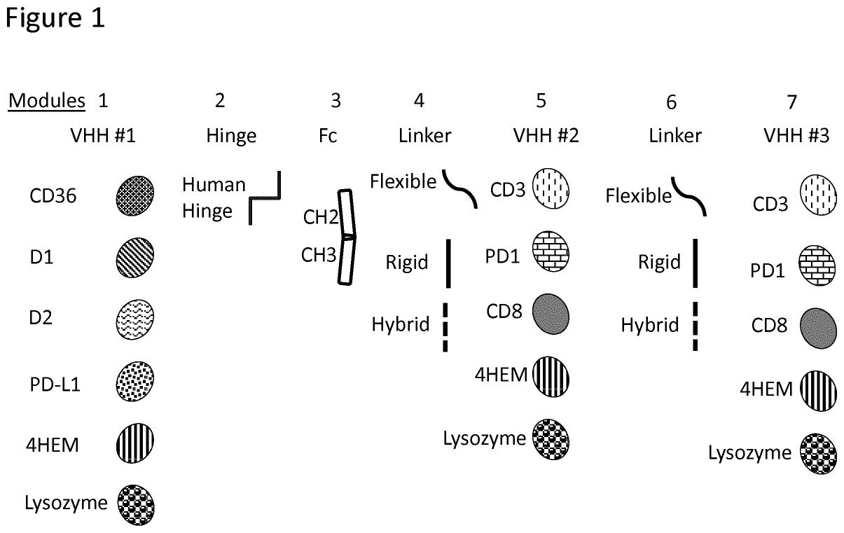

The different DNA modules were assembled using type IIs restriction cloning. When desired, the plasmid encoding the most 5′ DNA module may also contain a sequence encoding a signal peptide. Moreover, a sequence encoding a peptidic tag may be added to one or more of the DNA modules. Each DNA module is usually provided from a unique plasmid to allow design flexibility. FIG. 1 provides exemplary embodiments of various modules used in the polypeptides disclosed herein.

The polypeptides of Table 1, Table 2, Table 3 and Table 4 result from the assembly of 4-7 DNA modules each encoded by a unique plasmid.

Golden gate reaction was performed with the NEB® Golden Gate Assembly Mix (NEB1600). The final construct was assembled by ligation using 100 ng of each module which have been previously cloned into a vector lacking BsaI restrictions sites and 100 ng of the vector plasmid pNE-B340. The ligation reactions were done in the same tube using thermocycler, with 30 cycles of 5 minutes at 37° C., and 5 minutes at 16° C., then one incubation at 55° C. for 5 min. To reduce background colonies that do not contain the correct final product an additional digestion was performed on the ligation/digestion product using the BsaI-HFv2 (NEBR3733L) restriction enzyme.

All restriction enzyme digests were performed using enzymes from New England Biolabs (NEB), using the manufactures recommended protocol.

Transformation of E. coli

The ligation reaction mixture was used to transform E. coli (NEB® 5-alpha Competent E. coli, High Efficiency) in accordance with manufacturer's instructions. Briefly, 50 μl of E. coli competent cells was added to the ligation/digestion mixture and incubated on ice for 5 minutes. The cells were treated by heat shock for 30 seconds at 42° C. The cells were recovered by the addition of 350 μl SOC and incubated at 37° C. with 20 minutes shaking. Ten percent of the transformation reaction was plated on LB plates containing ampicillin (100 μg/mL).

### Screening of Colonies

Colonies were screened for the presence of correctly assembled DNA construct. Briefly, 5 to 12 colonies were picked and used to inoculate 2 ml LB with ampicillin. Cultures were grown overnight at 37° C. with shaking. Plasmids were extracted using Qiagen miniprep kit according to manufacturer's instructions. The plasmids were eluted with 50 μl elution buffer (10 mM Tris). Plasmids were quantified and analyzed by restriction digest with HindIII and EcoRI.

### Transfection and Expression of Polypeptides in ExpiCHO or in Expi293 Cells

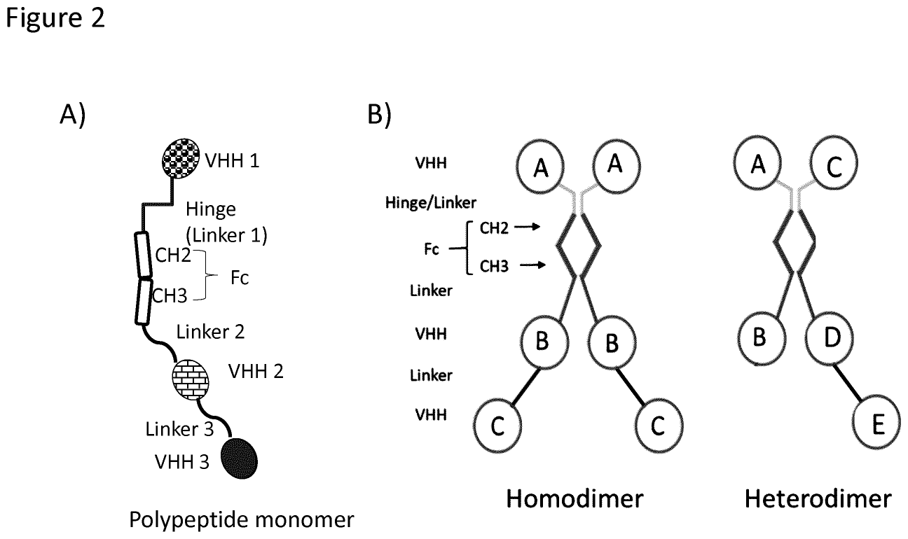

Protein dimers (e.g., homodimers or heterodimers) were expressed in 2.5 mL or 400 mL culture volume from the DNA construct using the ExpiCHO™ Expression System (Thermo Fisher, Cat. no. A29133) or the Expi293™ Expression System (Thermo Fisher, Cat. no. A14635). FIG. 2 illustrates exemplary configuration of protein dimers disclosed herein.

Briefly, freshly thawed CHO cells were allowed to recover in culture for two or more passages before transfection. Cells were then passaged every 3-4 days until they reach 4×10⁶-6×10⁶ cells/mL at which time they were diluted to 2×10⁵-3×10⁵ cells/mL in ExpiCHO™ Expression Medium pre-warmed to 37° C. The day prior to transfection, cells were diluted to 3×10⁶-4×10⁶ cells/mL and on the day of transfection, cells were further diluted to 6×10⁶ cells/mL. 1 μg of DNA/mL of culture volume was diluted with cold OptiPRO™ medium (100 μL for 2.5 mL of culture volume; 16 mL for 400 mL of culture volume). ExpiFectamine™ CHO Reagent (8 μL for 2.5 mL of culture volume; 1280 μL for 400 mL of culture volume) was added to medium containing DNA and incubated with ExpiFectamine™/DNA complexes at room temperature for 1-5 min. Then the DNA complex was transferred to culture (at 6×10⁶ cells/mL) while swirling. The cells were incubated at 37° C. under 8% CO₂ and 80% humidity with shaking (INFORS HT shaker, 125 rpm). 18-22h after onset of transfection, ExpiCHO™ feed (0.6 mL for 2.5 mL of culture volume; 96 mL for 400 mL of culture volume) and ExpiCHO™ enhancer (15 μL for 2.5 mL of culture volume; 2.4 mL for 400 mL of culture volume) were added to the cells. The cells were returned to INFORS HT incubator set at 37° C. under 8% CO₂ and 80% humidity with shaking at 125 rpm (25 mm orbit). 8 days post-transfection, supernatants were clarified by centrifugation at 4000×g for 30 min. Supernatants were filter-sterilized using a Nalgene™ Rapid-Flow™ Sterile Disposable Filter Units 1000 mL filter unit (Thermo Scientific, Cat. no. 567-0020) and were stored at 4° C. or frozen for later analysis.

Freshly thawed HEK293 cells were allowed to recover in culture for two or more passages before transfection. Cells were then passaged every 3-4 days until they reach 3×10⁶-5×10⁶ cells/ml at which time they were diluted to 3×10⁵-5×10⁵ cells/mL in Expi293™ Expression Medium pre-warmed to 37° C. The day prior to transfection, cells were diluted to 2.5×10⁶-3×10⁶ and on the day of transfection, cells were further diluted to 3×10⁶ viable cells/ml. 1 μg of DNA/mL of culture volume was diluted with Opti-MEM™ I Reduced Serum medium to get a final volume of 150 for 2.5 mL of culture volume and 24 mL for 400 mL of culture volume. ExpiFectamine™ 293 Reagent (8 μL for 2.5 mL of culture volume; 1.3 mL for 400 mL of culture volume) was added to medium Opti-MEM™ I Reduced Serum medium (140 μL for 2.5 mL of culture volume; 22.5 mL for 400 mL of culture volume) to incubate at room temperature for 5 minutes. Diluted ExpiFectamine™ was added to diluted DNA and incubate for 15 minutes at room temperature. ExpiFectamine™/DNA solution was transferred to culture drop by drop (at 3×10⁶ cells/ml) while swirling. The cells were incubated at 37° C. under 8% CO₂ and 80% humidity with overnight shaking (INFORS HT shaker, 125 rpm). 18-22h after onset of transfection, ExpiFectamine™ 293 Transfection Enhancer 1 (15 μL for 2.5 mL of culture volume; 2.4 mL for 400 mL of culture volume) and ExpiFectamine™ 293 Transfection Enhancer 2 (50 μL for 2.5 mL of culture volume; 24 mL for 400 mL of culture volume) were added to the cells. The cells were returned to INFORS HT incubator set at 37° C. under 8% CO₂ and 80% humidity with shaking at 125 rpm (25 mm orbit). 5 days post-transfection, supernatants were clarified by centrifugation at 4000×g for 30 min. Supernatants were filter-sterilized using a Nalgene™ Rapid-Flow™ Sterile Disposable Filter Units 1000 mL filter unit (Thermo Scientific, Cat. no. 567-0020) and were stored at 4° C. or frozen for later analysis.

### Purification

Proteins are purified using 3-mL MabSelect™ SuRe™ resin (GE Healthcare, Cat. No. 17-5438-02) with gravity columns or 40-mL MabSelect™ SuRe™ resin with AKTA PURE (GE Healthcare, Piscataway, N.J.) depending on the supernatant volume. Resin was incubated with 0.5 NaOH overnight and equilibrated with Tris-base buffer pH 7.4 (50 mM Tris-HCl, 150 mM NaCl, pH 7.4) prior injection. Supernatant was applied on gravity columns or the at 5 mL/min on 40-mL column. Resin column was washed with 3 CV (column volume) with Tris-base buffer pH 7.4 at flow rate of 10 mL/min. Protein was eluted with 3 CV of 0.1M citrate acid pH 3 at 10 mL/min. Fractions identified with protein from the visual output of the chromatogram (absorbance at 280 nm) were pooled together. Pooled fractions were neutralized with 1 M Tris-HCl pH 9 to achieve the pH˜5-6 before transferring into PBS (Phosphate-buffered saline) pH 6 buffer prepared from PBS 10X pH 7.2 (15 mM Potassium Phosphate monobasic 1552 mM Sodium Chloride 27 mM Sodium Phosphate dibasic, ThermoFisher, Cat. no. 70013073).

Buffer exchange was carried out by sample concentrators for proteins purified from gravity columns or either by dialysis or by desalting column for proteins purified from AKTA PURE. Proteins purified from gravity columns were concentrated with sample concentrator VivaSpin 2, 50 kDa MWCO (GE Healthcare, Cat. no. 28932257) by centrifugation at 3,500-4,000×g at 4° C. then, diluted with PBS pH 6 to achieve 4-fold and repeated until sample reached 200-fold. Dialysis was carried out in 4 L of PBS pH 6 overnight at 4° C. using 7 kDa molecular weight cut-off dialysis tubing (ThermoFisher, Cat. no. 68799). On the other hand, desalting column was incubated with 0.5 NaOH overnight and equilibrated with PBS pH 6. Volume of 15 mL of neutralized protein sample was loaded into the HiPrep 26/10 desalting column (GE Healthcare, Cat. no. 17-5087-02) at 0.5 mL/min then, protein was eluted with 2 CV of PBS pH 6. Loading and elution steps were repeated until no neutralized protein sample from elution of affinity column is left. Fractions identified with protein from the visual output of the chromatogram (absorbance at 280 nm) were pooled together.

Sample was filter-sterilized using a Nalgene™ Rapid-Flow™ Sterile Disposable Filter Units 150 mL filter unit (Thermo Scientific, Cat. no. 565-0010). Final protein sample was quantified by Pierce™ bicinchoninic acid Protein Assay kit (ThermoFisher, Cat. no. 23227) and tested for endotoxin level with Endosafe® LAL Reagent cartridges (Charles River Cat. no. PTS2005). Final protein sample was analyzed on SDS-PAGE gels under reducing or non-reducing conditions (see section SDS PAGE and Western Blotting).

### SDS PAGE and Western Blotting

Samples were prepared for SDS-PAGE analysis under reducing or non-reducing conditions by heating with NuPAGE™ LDS Sample Buffer (ThermoFisher Cat. no NP0007) with NuPAGE™ Sample Reducing Agent (ThermoFisher Cat. no. NP0004) or without agent reducing buffer. Samples were denatured by heating at 70° C. for 10 minutes. Samples (16 μL) were loaded onto 3-8% Tris-Acetate mini-gels (1.5 mm, 15 wells) alongside a BSA standard. Electrophoresis was conducted using a X-Cell SureLock™ mini-gel device at 125 volts for approximately 1 hour. Gels were stained using GelCode™ staining reagent (Thermo Fisher, Cat. no. 24594).

For Western blots analysis, proteins were transferred to nitrocellulose membranes using the iBlot™ system (Thermo Fisher, Cat. no. IB301031) according to the manufacturer's instructions.

Detection of the His epitope tag was carried out with the Anti-Penta His-HRP antibody. Briefly, membranes were blocked by incubation in 20 ml in Qiagen blocking buffer (Qiagen, Cat. no. 1018862) for 1 hour at room temperature with shaking, followed by incubation in 20 ml in Starting Blocking™ T20 (PBS) Blocking (Thermo Fisher, Cat. no. 37528) for 1 hour at room temperature with shaking. Membranes were washed three times for 10 minutes with 1× TBS Tween™-20. Membranes were incubated with Anti-Penta His-HRP (Qiagen, Cat. no. 1014992) previously diluted 1:2000 in blocking buffer for 1 hour at room temperature with shaking. Membranes were washed three times for 10 minutes with 1×TBS Tween™-20. The signal was visualized using of Super Signal™ West Pico PLUS (Thermo Fisher, Cat. no. 34080) according to the manufacturer's instructions. Images were recorded using the Azure Biosystem imaging system.

### Example 2: Synthesis and Production of Polypeptides and Polypeptide Dimers

Single domain antibodies were generated by immunization of camels or llama or were obtained by in vitro synthesis.

DNA constructs containing various DNA modules including the antigen binding domains of VHHs were generated, polypeptides were expressed, and polypeptides or protein dimers isolated and analyzed using the methods described herein.

Table 1, Table 2, Table 3 and Table 4 provide exemplary embodiments of polypeptides generated by assembly of various modules.

The polypeptides of Table 1 and Table 2 contain a natural dimerization domain (wild type CH2-CH3) that allows dimerization of polypeptides in transfected cells. The polypeptide chains of Table 1 and Table 2 therefore naturally form a homodimer comprising two identical arms.

The polypeptides of Table 3 and Table 4 contain a mutated dimerization domain (wild type CH2 and mutated CH3) that favorizes the formation of heterodimers. More particularly, the polypeptides of Table 3 and Table 4 have the possibility of forming a homodimer when expressed alone or to form a heterodimer when expressed with a complementary chain. Heterodimers were particularly made by transfection of the set of chains (Chain A and Chain B) listed in Table 3 or Table 4.

In some experiments, the Applicant used either proof of principle (POP) antigen binding domains including those of anti-CD3 (α-CD3: SEQ ID NO:20), anti-PD1 (α-PD1; SEQ ID NO:21), anti-hen egg-white lysozyme (α-HEWL: SEQ ID NO:22), anti-4HEM (α-4HEM: SEQ ID NO:23), or anti-PDL1 (α-PDL1: SEQ ID NO:24). In other experiments, the Applicant used antigen binding domains obtained from VHHs raised against tumor-antigens.

As demonstrated herein, the sequence of the antigen binding domains may be selected based on the desired specificity and valency and their positions within the polypeptide chain may vary. For example, each of the antigen binding domain may be permutated or exchanged by another having different sequence and/or specificity. Moreover, additional antigen binding domain and linkers may be added at one or both of the N- or C-terminal end of the multivalent protein and extended.

The code name α-DRD2.1, α-DRD2.2, α-DRD2.3 and α-DRD2.4, α-DRD1.1, α-DRD1.2, α-DRD1.3 and α-DRD1.4 or α-CD36.1, α-CD36.2, α-CD36.3 and α-CD36.4 used herein represent VHHs having different sets of complementary determining regions.

Proof of principle homodimers were generated by transfecting cells with a plasmid expressing the polypeptides identified by the code names KB015, KB016, KB017, KB018, KB019, KB020, KB021, KB022, KB023, KB024, KB025, KB026, KB027, KB028, KB029, KB030, KB031, KB032, KB033, KB034, KB035, KB036, KB037, KB038, KB039, KB040, KB041, KB042 and comprising the amino acid sequence indicated in Table 1.

TABLE 1
Exemplary POP polypeptides containing CH2-CH3 domain of a natural antibody

| | | Module 1 | Module 2 | Module 3 | Module 4 | Module 5 | Module 6 | Module 7 |
|---|---|---|---|---|---|---|---|---|
| Code Name | Specificity | VHH 1 | Linker 1 | Fc | Linker 2 | VHH 2 | Linker 3 | VHH 3 |
| KB015 | PDL1, | α-PDL1 | SEQ ID | SEQ ID | SEQ ID | α-CD3 | SEQ ID | α-PD1 |
| | CD3, PD1 | SEQ ID | NO: 1 | NO: 16 | NO: 2 | SEQ ID | NO: 4 | SEQ ID |
| | | NO: 24 | | | | NO: 20 | | NO: 21 |
| KB070 | PDL1, | α-PDL1 | SEQ ID | SEQ ID | SEQ ID | α-CD3 | SEQ ID | α-PD1 |
| | CD3, PD1 | SEQ ID | NO: 1 | NO: 16 | NO: 9 | SEQ ID | NO: 4 | SEQ ID |
| | | NO: 24 | | | | NO: 20 | | NO: 21 |
| KB016 | PDL1, | α-PDL1 | SEQ ID | SEQ ID | SEQ ID | α-PD1 | SEQ ID | α-CD3 |
| | CD3, PD1 | SEQ ID | NO: 1 | NO: 16 | NO: 2 | SEQ ID | NO: 4 | SEQ ID |
| | | NO: 24 | | | | NO: 21 | | NO: 20 |
| KB071 | PDL1, | α-PDL1 | SEQ ID | SEQ ID | SEQ ID | α-PD1 | SEQ ID | α-CD3 |
| | CD3, PD1 | SEQ ID | NO: 1 | NO: 16 | NO: 9 | SEQ ID | NO: 4 | SEQ ID |
| | | NO: 24 | | | | NO: 21 | | NO: 20 |
| KB017 | 4HEM, | SEQ ID | SEQ ID | SEQ ID | SEQ ID | SEQ ID | SEQ ID | SEQ ID |
| | 4HEM, | NO: 23 | NO: 1 | NO: 16 | NO: 2 | NO: 23 | NO: 4 | NO: 23 |
| | 4HEM | | | | | | | |
| KB018 | HEWL, | SEQ ID | SEQ ID | SEQ ID | SEQ ID | SEQ ID | SEQ ID | SEQ ID |
| | HEWL, | NO: 22 | NO: 1 | NO: 16 | NO: 2 | NO: 22 | NO: 4 | NO: 22 |
| | HEWL | | | | | | | |
| KB019 | 4HEM, | SEQ ID | SEQ ID | SEQ ID | SEQ ID | α-CD3 | SEQ ID | α-PD1 |
| | CD3, PD1 | NO: 23 | NO: 1 | NO: 16 | NO: 2 | SEQ ID | NO: 4 | SEQ ID |
| | | | | | | NO: 20 | | NO: 21 |
| KB020 | PDL1, | α-PDL1 | SEQ ID | SEQ ID | SEQ ID | SEQ ID | SEQ ID | SEQ ID |
| | 4HEM, | SEQ ID | NO: 1 | NO: 16 | NO: 14 | NO: 23 | NO: 4 | NO: 23 |
| | 4HEM | NO: 24 | | | | | | |
| KB021 | 4HEM, | SEQ ID | SEQ ID | SEQ ID | SEQ ID | α-CD3 | SEQ ID | SEQ ID |
| | CD3, | NO: 23 | NO: 1 | NO: 16 | NO: 14 | SEQ ID | NO: 4 | NO: 23 |
| | 4HEM | | | | | NO: 20 | | |
| KB022 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 14 | NO: 23 | NO: 4 | SEQ ID |
| | PD1 | | | | | | | NO: 21 |
| KB023 | 4HEM, | SEQ ID | SEQ ID | SEQ ID | SEQ ID | SEQ ID | SEQ ID | SEQ ID |
| | 4HEM, | NO: 23 | NO: 1 | NO: 16 | NO: 14 | NO: 23 | NO: 4 | NO: 23 |
| | 4HEM | | | | | | | |
| KB024 | 4HEM, | SEQ ID | SEQ ID | SEQ ID | SEQ ID | SEQ ID | SEQ ID | SEQ ID |
| | PD1, | NO: 23 | NO: 1 | NO: 16 | NO: 3 | NO: 21 | NO: 4 | NO: 23 |
| | 4HEM | | | | | | | |
| KB025 | 4HEM, | SEQ ID | SEQ ID | SEQ ID | SEQ ID | α-PD1 | SEQ ID | SEQ ID |
| | PD1, | NO: 23 | NO: 1 | NO: 16 | NO: 5 | SEQ ID | NO: 4 | NO: 23 |
| | 4HEM | | | | | NO: 21 | | |
| KB026 | 4HEM, | SEQ ID | SEQ ID | SEQ ID | SEQ ID | α-PD1 | SEQ ID | SEQ ID |
| | PD1, | NO: 23 | NO: 1 | NO: 16 | NO: 6 | SEQ ID | NO: 4 | NO: 23 |
| | 4HEM | | | | | NO: 21 | | |
| KB027 | 4HEM, | SEQ ID | SEQ ID | SEQ ID | SEQ ID | α-PD1 | SEQ ID | SEQ ID |
| | PD1, | NO: 23 | NO: 1 | NO: 16 | NO: 12 | SEQ ID | NO: 4 | NO: 23 |
| | 4HEM | | | | | NO: 21 | | |
| KB028 | 4HEM, | SEQ ID | SEQ ID | SEQ ID | SEQ ID | α-PD1 | SEQ ID | SEQ ID |
| | PD1, | NO: 23 | NO: 1 | NO: 16 | NO: 13 | SEQ ID | NO: 4 | NO: 23 |
| | 4HEM | | | | | NO: 21 | | |
| KB029 | 4HEM, | SEQ ID | SEQ ID | SEQ ID | SEQ ID | α-PD1 | SEQ ID | SEQ ID |
| | PD1, | NO: 23 | NO: 1 | NO: 16 | NO: 14 | SEQ ID | NO: 4 | NO: 23 |
| | 4HEM | | | | | NO: 21 | | |
| KB030 | 4HEM, | SEQ ID | SEQ ID | SEQ ID | SEQ ID | α-PD1 | SEQ ID | SEQ ID |
| | PD1, | NO: 23 | NO: 1 | NO: 16 | NO: 9 | SEQ ID | NO: 4 | NO: 23 |
| | 4HEM | | | | | NO: 21 | | |
| KB031 | 4HEM, | SEQ ID | SEQ ID | SEQ ID | SEQ ID | α-PD1 | SEQ ID | SEQ ID |
| | PD1, | NO: 23 | NO: 1 | NO: 16 | NO: 10 | SEQ ID | NO: 4 | NO: 23 |
| | 4HEM | | | | | NO: 21 | | |
| KB032 | 4HEM, | SEQ ID | SEQ ID | SEQ ID | SEQ ID | α-PD1 | SEQ ID | SEQ ID |
| | PD1, | NO: 23 | NO: 1 | NO: 16 | NO: 8 | SEQ ID | NO: 4 | NO: 23 |
| | 4HEM | | | | | NO: 21 | | |
| KB033 | PD1 | SEQ ID | SEQ ID | SEQ ID | — | — | — | — |
| | | NO: 21 | NO: 1 | NO: 16 | | | | |
| KB034 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 3 | SEQ ID |
| | PD1 | | | | | | | NO: 21 |
| KB035 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 4 | SEQ ID |
| | PD1 | | | | | | | NO: 21 |
| KB036 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 6 | SEQ ID |
| | PD1 | | | | | | | NO: 21 |
| KB037 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 13 | SEQ ID |
| | PD1 | | | | | | | NO: 21 |
| KB038 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 14 | SEQ ID |
| | PD1 | | | | | | | NO: 21 |
| KB039 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 9 | SEQ ID |
| | PD1 | | | | | | | NO: 21 |
| KB040 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 10 | SEQ ID |
| | PD1 | | | | | | | NO: 21 |
| KB041 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 7 | SEQ ID |
| | PD1 | | | | | | (n = 1) | NO: 21 |
| KB042 | 4HEM, | SEQ ID | SEQ ID | SEQ ID | SEQ ID | SEQ ID | SEQ ID | α-PD1 |
| | 4HEM, | NO: 23 | NO: 1 | NO: 16 | NO: 2 | NO: 23 | NO: 8 | SEQ ID |
| | PD1 | | | | | | | NO: 21 |

Exemplary, tumor-specific polypeptides were generated by replacing the VHH 1 portion of the polypeptides of Table 1 (e.g., KB015 or KB016) with the antigen binding domain of VHHs generated against dopamine receptor 2 (DRD2), dopamine receptor 1 (DRD1) or CD36 and/or by replacing the Fc portion with corresponding CH2-CH3 domains of IgG4. In some experiments, the natural CH2-CH3 dimerization domain of IgG4 appears to function as well as the IgG1 natural CH2-CH3 domain.

Tumor-specific homodimers were generated by transfecting cells with a plasmid expressing the polypeptides identified by the code names KB001, KB003, KB004, KB005, KB006, KB007, KB008, KB009, KB010, KB011, KB012, KB013, KB014 comprising the amino acid sequence indicated in Table 2.

TABLE 2
Exemplary tumor-specific polypeptides containing CH2-CH3 domain of a natural antibody.

| | | Module 1 | Module 2 | Module 3 | Module 4 | Module 5 | Module 6 | Module 7 |
|---|---|---|---|---|---|---|---|---|
| Code Name | Specificity | VHH 1 | Linker 1 | Fc | Linker 2 | VHH 2 | Linker 3 | VHH 3 |
| KB001 | DRD2, | α- | SEQ ID | SEQ ID | SEQ ID | α-CD3 | SEQ ID | α-PD1 |
| | CD3, PD1 | DRD2.1 | NO: 1 | NO: 16 | NO: 2 | SEQ ID | NO: 4 | SEQ ID |
| | | | | | | NO: 20 | | NO: 21 |
| KB059 | DRD2, | α- | SEQ ID | SEQ ID | SEQ ID | α-CD3 | SEQ ID | α-PD1 |
| | CD3, PD1 | DRD2.1 | NO: 1 | NO: 16 | NO: 9 | SEQ ID | NO: 4 | SEQ ID |
| | | | | | | NO: 20 | | NO: 21 |
| KB060 | DRD2, | α- | SEQ ID | SEQ ID | SEQ ID | α-PD1 | SEQ ID | α-CD3 |
| | PD1, CD3 | DRD2.1 | NO: 1 | NO: 16 | NO: 2 | SEQ ID | NO: 4 | SEQ ID |
| | | | | | | NO: 21 | | NO: 20 |
| KB061 | DRD2, | α- | SEQ ID | SEQ ID | SEQ ID | α-PD1 | SEQ ID | α-CD3 |
| | PD1, CD3 | DRD2.1 | NO: 1 | NO: 16 | NO: 9 | SEQ ID | NO: 4 | SEQ ID |
| | | | | | | NO: 21 | | NO: 20 |
| KB003 | DRD2, | α- | SEQ ID | SEQ ID | SEQ ID | α-CD3 | SEQ ID | α-PD1 |
| | CD3, PD1 | DRD2.2 | NO: 1 | NO: 16 | NO: 2 | SEQ ID | NO: 4 | SEQ ID |
| | | | | | | NO: 20 | | NO: 21 |
| KB004 | DRD2, | α- | SEQ ID | SEQ ID | SEQ ID | α-CD3 | SEQ ID | α-PD1 |
| | CD3, PD1 | DRD2.3 | NO: 1 | NO: 16 | NO: 2 | SEQ ID | NO: 4 | SEQ ID |
| | | | | | | NO: 20 | | NO: 21 |
| KB005 | DRD2, | α- | SEQ ID | SEQ ID | SEQ ID | α-CD3 | SEQ ID | α-PD1 |
| | CD3, PD1 | DRD2.4 | NO: 1 | NO: 16 | NO: 2 | SEQ ID | NO: 4 | SEQ ID |
| | | | | | | NO: 20 | | NO: 21 |
| KB044 | DRD2, | α- | SEQ ID | SEQ ID | SEQ ID | α-PD1 | SEQ ID | α-CD3 |
| | PD1, CD3 | DRD2.1 | NO: 1 | NO: 25 | NO: 2 | SEQ ID | NO: 4 | SEQ ID |
| | | | | | | NO: 21 | | NO: 20 |
| KB043 | DRD2, | α- | SEQ ID | SEQ ID | SEQ ID | α-PD1 | SEQ ID | α-CD3 |
| | PD1, CD3 | DRD2.1 | NO: 1 | NO: 26 | NO: 2 | SEQ ID | NO: 4 | SEQ ID |
| | | | | | | NO: 21 | | NO: 20 |
| KB006 | PDL1, | α- | SEQ ID | SEQ ID | SEQ ID | α-CD3 | SEQ ID | α-PD1 |
| | CD3, PD1 | PDL1.1 | NO: 1 | NO: 16 | NO: 2 | SEQ ID | NO: 4 | SEQ ID |
| | | | | | | NO: 20 | | NO: 21 |
| KB035 | DRD1, | α- | SEQ ID | SEQ ID | SEQ ID | α-CD3 | SEQ ID | α-PD1 |
| | CD3, PD1 | DRD1.1 | NO: 1 | NO: 16 | NO: 2 | SEQ ID | NO: 4 | SEQ ID |
| | | | | | | NO: 20 | | NO: 21 |
| KB062 | DRD1, | α- | SEQ ID | SEQ ID | SEQ ID | α-CD3 | SEQ ID | α-PD1 |
| | CD3, PD1 | DRD1.1 | NO: 1 | NO: 16 | NO: 9 | SEQ ID | NO: 4 | SEQ ID |
| | | | | | | NO: 20 | | NO: 21 |
| KB063 | DRD1, | α- | SEQ ID | SEQ ID | SEQ ID | α-PD1 | SEQ ID | α-CD3 |
| | PD1, CD3 | DRD1.1 | NO: 1 | NO: 16 | NO: 2 | SEQ ID | NO: 4 | SEQ ID |
| | | | | | | NO: 21 | | NO: 20 |
| KB064 | DRD1, | α- | SEQ ID | SEQ ID | SEQ ID | α-PD1 | SEQ ID | α-CD3 |
| | PD1, CD3 | DRD1.1 | NO: 1 | NO: 16 | NO: 9 | SEQ ID | NO: 4 | SEQ ID |
| | | | | | | NO: 21 | | NO: 20 |
| KB007 | DRD1, | α- | SEQ ID | SEQ ID | SEQ ID | SEQ ID | SEQ ID | α-CD3 |
| | CD3, PD1 | DRD1.1 | NO: 1 | NO: 16 | NO: 2 | NO: 21 | NO: 4 | SEQ ID |
| | | | | | | | | NO: 20 |
| KB008 | DRD1, | α- | SEQ ID | SEQ ID | SEQ ID | α-CD3 | SEQ ID | α-PD1 |
| | CD3, PD1 | DRD1.2 | NO: 1 | NO: 16 | NO: 2 | SEQ ID | NO: 4 | SEQ ID |
| | | | | | | NO: 20 | | NO: 21 |
| KB009 | DRD1, | α- | SEQ ID | SEQ ID | SEQ ID | α-CD3 | SEQ ID | α-PD1 |
| | CD3, PD1 | DRD1.3 | NO: 1 | NO: 16 | NO: 2 | SEQ ID | NO: 4 | SEQ ID |
| | | | | | | NO: 20 | | NO: 21 |
| KB010 | DRD1, | α- | SEQ ID | SEQ ID | SEQ ID | α-CD3 | SEQ ID | α-PD1 |
| | CD3, PD1 | DRD1.4 | NO: 1 | NO: 16 | NO: 2 | SEQ ID | NO: 4 | SEQ ID |
| | | | | | | NO: 20 | | NO: 21 |
| KB011 | CD36, | α- | SEQ ID | SEQ ID | SEQ ID | α-CD3 | SEQ ID | α-PD1 |
| | CD3, PD1 | CD36.1 | NO: 1 | NO: 16 | NO: 2 | SEQ ID | NO: 4 | SEQ ID |
| | | | | | | NO: 20 | | NO: 21 |
| KB065 | CD36, | α- | SEQ ID | SEQ ID | SEQ ID | α-CD3 | SEQ ID | α-PD1 |
| | CD3, PD1 | CD36.1 | NO: 1 | NO: 16 | NO: 9 | SEQ ID | NO: 4 | SEQ ID |
| | | | | | | NO: 20 | | NO: 21 |
| KB066 | CD36, | α- | SEQ ID | SEQ ID | SEQ ID | α-PD1 | SEQ ID | α-CD3 |
| | CD3, PD1 | CD36.1 | NO: 1 | NO: 16 | NO: 2 | SEQ ID | NO: 4 | SEQ ID |
| | | | | | | NO: 21 | | NO: 20 |
| KB067 | CD36, | α- | SEQ ID | SEQ ID | SEQ ID | α-PD1 | SEQ ID | α-CD3 |
| | CD3, PD1 | CD36.1 | NO: 1 | NO: 16 | NO: 9 | SEQ ID | NO: 4 | SEQ ID |
| | | | | | | NO: 21 | | NO: 20 |
| KB012 | CD36, | α- | SEQ ID | SEQ ID | SEQ ID | α-CD3 | SEQ ID | α-PD1 |
| | CD3, PD1 | CD36.2 | NO: 1 | NO: 16 | NO: 2 | SEQ ID | NO: 4 | SEQ ID |
| | | | | | | NO: 20 | | NO: 21 |
| KB013 | CD36, | α- | SEQ ID | SEQ ID | SEQ ID | α-CD3 | SEQ ID | α-PD1 |
| | CD3, PD1 | CD36.3 | NO: 1 | NO: 16 | NO: 2 | SEQ ID | NO: 4 | SEQ ID |
| | | | | | | NO: 20 | | NO: 21 |
| KB014 | CD36, | α- | SEQ ID | SEQ ID | SEQ ID | α-CD3 | SEQ ID | α-PD1 |
| | CD3, PD1 | CD36.4 | NO: 1 | NO: 16 | NO: 2 | SEQ ID | NO: 4 | SEQ ID |
| | | | | | | NO: 20 | | NO: 21 |

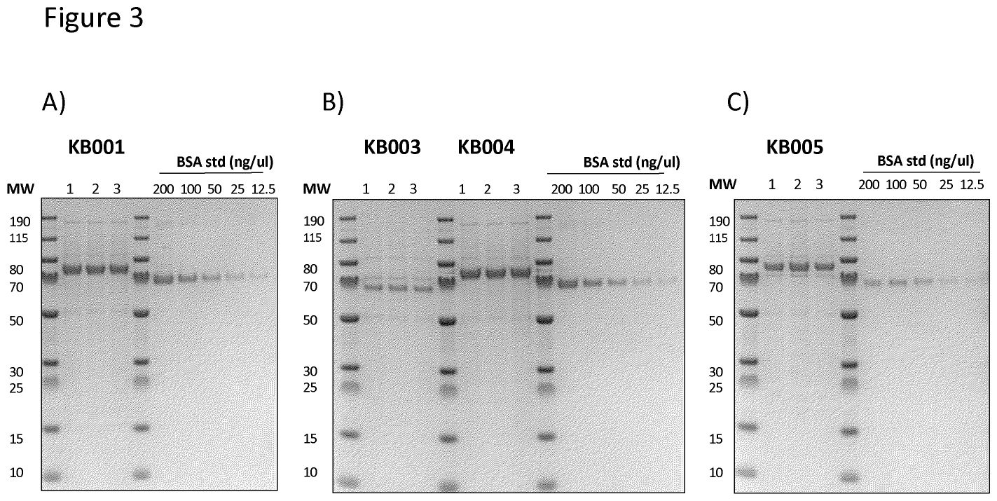

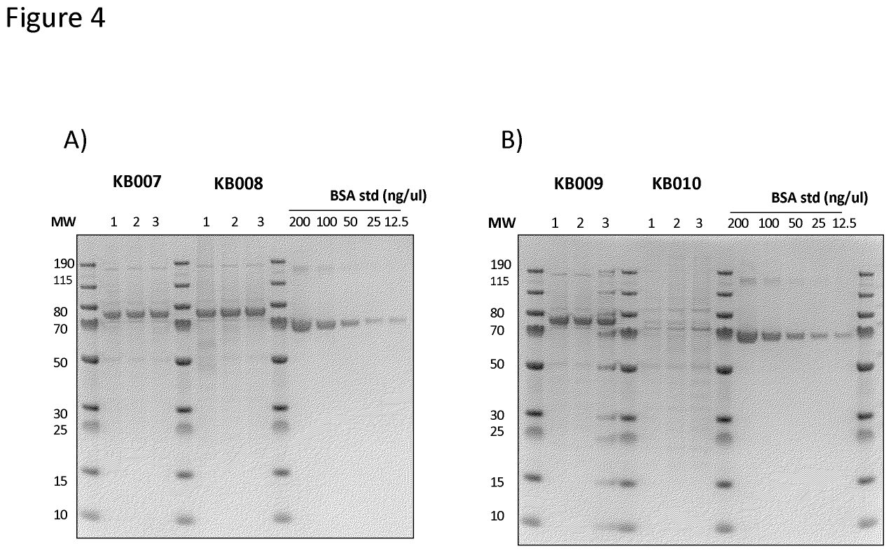

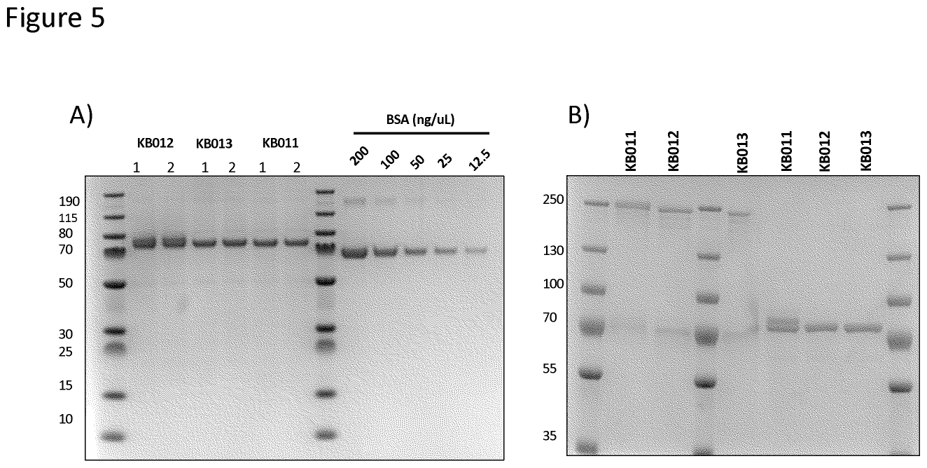

As can be seen from SDS-PAGE analysis presented in FIGS. 3-5, the polypeptides KB001, KB003, KB004, KB005 (FIG. 3), KB007, KB008, KB009, KB010 (FIG. 4), KB012, KB013 and KB011 (FIG. 5) are successfully expressed in mammalian cells and have the expected molecular weight.

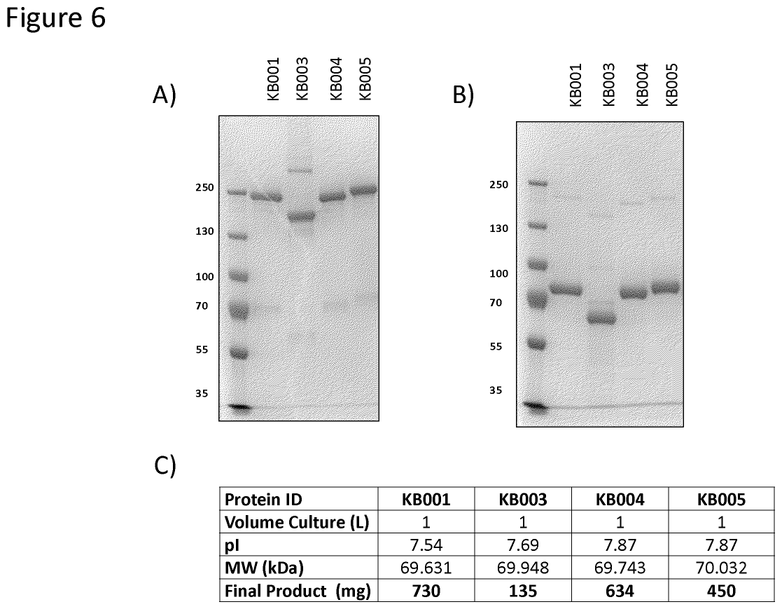

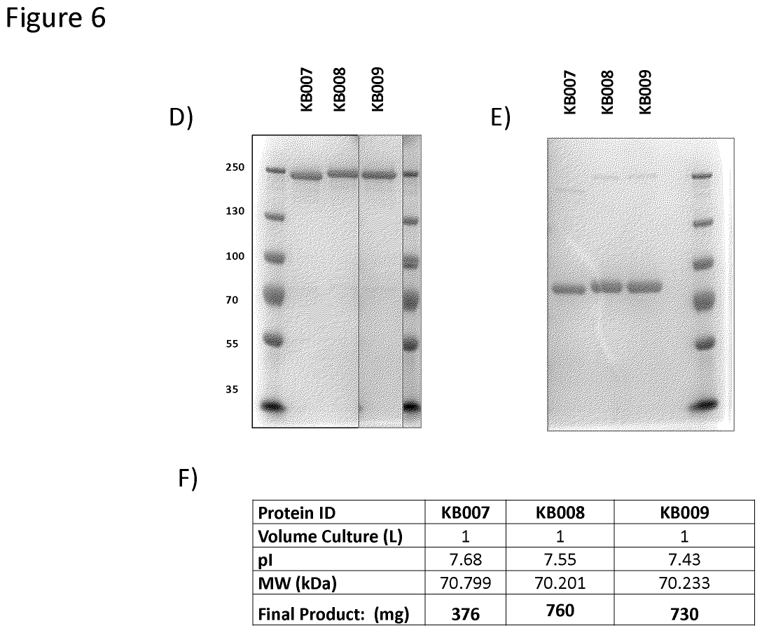

Moreover, results of FIG. 6A, FIG. 6B, FIG. 6D and FIG. 6E show that protein dimers made from KB001, KB003, KB004, KB005, KB008, KB009 and KB007 can be purified according to purification process described in the method section.

### Example 3: In Vitro Testing

### In Vitro Cytotoxicity

The cytotoxicity of the polypeptides and protein dimers of the present disclosure was assessed in in vitro experiments.

Briefly, tumor cells were resuspended in cell culture medium to yield 5×10⁶ cells/ml. The cells were labelled with CellTrace™ Violet solution, then incubated at 37° C. for 10 minutes. The CellTrace™ Violet-labelled tumor cells were mixed with PBMCs at a ratio of 1:10. The cells were treated with the protein dimers or with the controls at the indicated concentration. Cells were incubated at 4° C. for 10 minutes on ice with 7-Amino-Actinomycin D (7-AAD) solution which stains dead cells. The number of viable cells was determined by Flow Cytometry to compare the percentage of cells stained with 7-AAD to the total number of cells labelled with CellTrace™ Violet. The results are presented as percentage of dead cells.

### FACS Binding Assay on Jurkat Cells

The binding of the protein dimers to the Jurkat human tumor cell line was assessed by Flow Cytometry. Briefly, Jurkat cells were pre-stimulated with an anti-CD3 antibody (OKT3) overnight. Non-specific binding was blocked by incubating the cells in blocking buffer for 10 minutes at 4° C. A solution containing protein dimers was added to the cells and incubated at 4° C. for 20 minutes. After incubation, cells were washed three times with FACS staining buffer. A fluorescent-labelled antibody targeting the Fc region of the polypeptides was added and the cells were incubated on ice for 20 minutes in the dark. The cells were again washed three times with FACS buffer, resuspended in 100 ul of FACS staining buffer and analyzed using a BD FACSCanto™ II Flow cytometer (BD Bioscience).

### ELISA Binding Assay

The binding of protein dimers was determined by Proteoliposome-ELISA. Briefly, proteoliposomes containing the target protein or peptide or empty liposomes were coated on a 96-well plate. The plate was covered and left at 4° C. overnight. The next day the plate was washed once with PBS and blocked with blocking buffer for 1 hour at room temperature. The protein dimers were tested at the indicated concentration, diluted in the blocking buffer and incubate for 1 hour at 37° C. After incubation, the wells were washed three times with PBS. The wells were incubated for 1 hour at room temperature with anti-IgG1-HRP diluted at 1:5000 in the blocking buffer then washed three times with PBS. The signal was developed with SuperSignal™ ELISA Pico Chemiluminescent Substrate. The plate was read on a SpectraMax™ i3x Multi-Mode Microplate Reader (Molecular Devices).

### In Vitro Viability Assay

The ability of protein dimers to decrease the viability of cells was assessed in vitro using the CellTiter-Fluor™ Cell Viability Assay (Promega) according to manufacturer's instructions and was compared with that of the negative control and the vehicle-treated (PBS) control.

### Results for POP Polypeptides and Protein Dimers

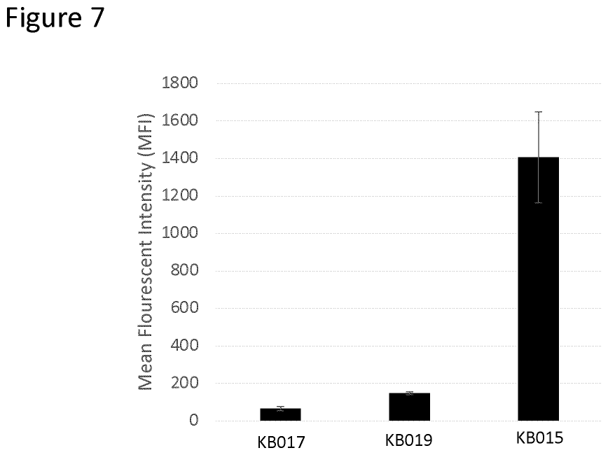

Exemplary results of experiments carried out with POP polypeptides and homodimers are presented in FIGS. 7 to 12.

### Binding Assays

The binding of homodimers made from the polypeptides KB017 (negative control), KB019 (targeting CD3 and PD1), or KB015 (targeting PDL1, CD3, and PD1) to Jurkat human tumor cell line was assessed by Flow Cytometry as indicated above.

Results of this experiment are presented in FIG. 7 and show that homodimers containing anti-PD1, anti-CD3 and anti-PDL1 VHHs have significantly higher binding than multivalent protein dimers with only anti-PD1, anti-CD3

### In Vitro Cytotoxicity

Using the cytotoxicity assay described above, the anti-tumor effect of homodimers made from the polypeptides KB019 or KB015 was assessed on the tumor cell line OCI-AML3.

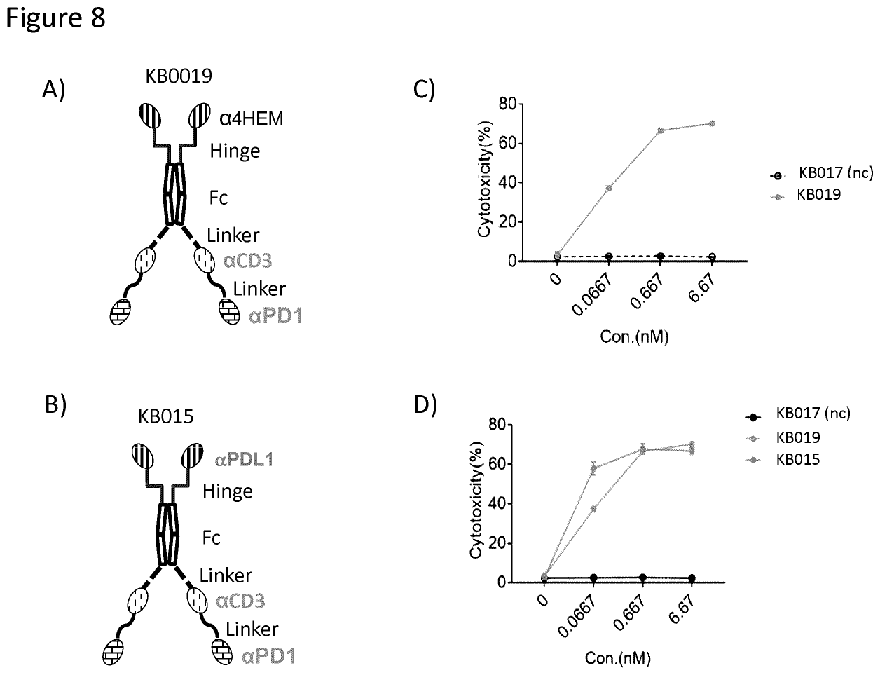

In a first set of experiments, OCI-AML3 cells were treated with either homodimers made from the KB017 polypeptide (negative control) or homodimers made from the KB019 polypeptide (targeting CD3 and PD1 FIG. 8A and FIG. 8B) at final concentration of 0 nM, 0.0667 nM, 0.667 nM, and 6.67 nM. After incubation, the cells were stained, and the percentage of dead cells was compared to that of viable cells.

Results of this experiment are presented at FIG. 8C and show that homodimers made from the KB019 polypeptide efficiently target and kill OCI-AML3 cells. Therefore, VHH targeting CD3 and PD1 confer functional activity to molecule in vitro.

In a second set of experiments, the anti-tumor effect of homodimers made from the trispecific KB015 polypeptide (targeting PDL1, CD3 and PD1: FIG. 8B) was compared to that of homodimers made from the KB019 polypeptide (FIG. 8A). Briefly, OCI-AML3 cells were treated with homodimers made from the KB017, KB019 or KB015 polypeptide at final concentration of 0 nM, 0.0667 nM, 0.667 nM, and 6.67 nM. After incubation, the cells were stained, and the percentage of dead cells was compared to that of viable cells.

Results of this experiment are presented at FIG. 8C and FIG. 8D and show that homodimers made from the KB017 polypeptide efficiently target and kill OCI-AML3 cells and that the addition of VHH targeting PDL1 enhances the functional activity of the multivalent protein dimer in vitro.

In a third set of experiments, the anti-tumor effect of homodimers made from the KB015 polypeptide was compared with that of homodimers made from the KB016 polypeptide which contains the same VHHs but at different position. Briefly, OCI-ML3 cells were treated each of the homodimers or with homodimers made from the KB018 polypeptide (negative control) at final concentration of 0 nM, 0.007 nM, 0.07 nM, 0.7 nM, and 7 nM for 48 hours. After incubation, the cells were stained, and the percentage of dead cells was compared to that of viable cells.

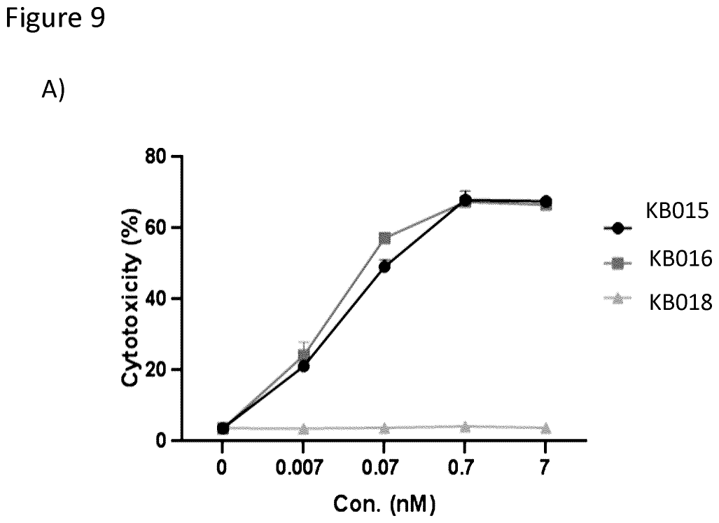

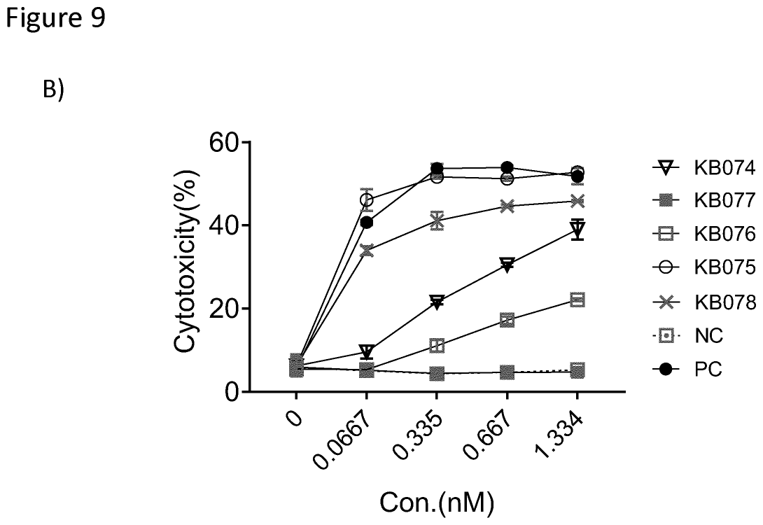

Results of this experiment are presented at FIG. 9A and show that changing the position of the anti-PD-1 and anti-CD3 VHHs does not affect cytotoxicity of the protein dimers.

In another experiment, tetraspecific polypeptides were generated and tested. These exemplary tetraspecific polypeptides comprise two VHH domains at the N-terminal of the dimerization domain and two VHH domains at the C-terminal of the dimerization domain.

Briefly, tetraspecific polypeptides were constructed with either four functionally active domains (targeting CD36, PDL1, CD3, and PD1: KB078), three functionally active domains (targeting PDL1, CD3, and PD1: KB075) or two functionally active domains (targeting CD3 and PD1: KB076) or a negative control protein with no functionally active domain (KB077). These molecules were compared with a set of trispecific polypeptides containing either three functionally active domains (targeting PDL1, CD3, and PD1) or two functionally active domains (targeting CD3 and PD1) or a negative control protein with no functionally active domain.

Briefly, target OCI-AML3 tumor cells were pre-labelled with Cell Trace Violet then treated with human PBMC effector cells in the presence of the tetraspecific polypeptides, the trispecific polypeptides at final concentration of 0 nM, 0.0667 nM, 0.335 nM, 0.667 nM, and 1.334 nM. After incubation, the dead cells were stained with 7-AAD, and the cytotoxicity was calculated as the percentage of dead cells was compared with the percentage of viable OCI-AML3 cells.

Results of this experiment are presented in FIG. 9B and show that dimers made from tetraspecific polypeptides efficiently target and kill OCI-AML3 cells.

In another experiment, the anti-tumor effect of homodimers made from the KB015 polypeptide was compared with that of homodimers made from polypeptides containing only one active VHH (KB020, KB021 or KB022) or with the combination of the three corresponding single domain antibody-Fc proteins; KB045 (anti-PDL1 VHH-Fc), KB046 (anti-CD3 VHH-Fc), KB033 (anti-PD1 VHH-Fc). Briefly, THP-1 cells were treated with the homodimers or with a combination of three VHH-Fc proteins or with negative control homodimers made from the KB023 polypeptide at final concentration of 0 nM, 0.007 nM, 0.07 nM, 0.7 nM, and 7 nM. After incubation, the cells were stained, and the percentage of dead cells was compared to that of viable cells.

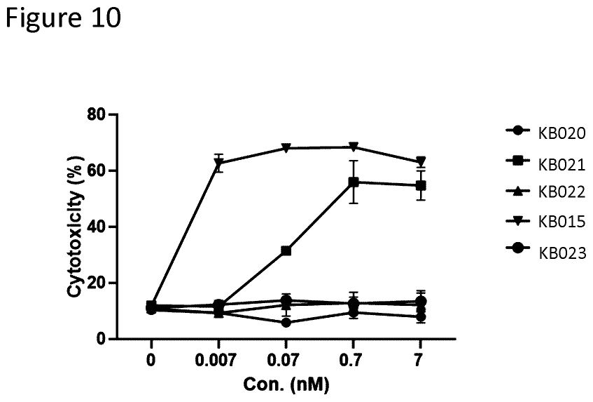

The results presented in FIG. 10 show that homodimers made from KB015 polypeptides have greater cytotoxicity than molecules containing only one VHH against PD-L1, CD3 or PD-1.

### Analysis of Linkers

The effect of the linker's sequence and position in the polypeptides was investigated.

Briefly, trivalent polypeptides containing the same VHHs with variation in the linker sequence were tested for their binding to recombinant protein PD-1. Construct KB033 which is an anti-PD1 VHH Fc protein was used as a positive control. Recombinant protein PD-1 was coated in 96-well plates at 1 μg/ml overnight. Plates were washed 3 times before blocking with 1% BSA. Polypeptides with different dilutions were added into the plates and incubated at room temperature for 1 hour. After washing 3 times, the secondary antibody anti-human IgG1-HRP was added at 0.2 μg/ml. 1 hour later, super signal ELISA Pico chemiluminescent substrate was added for detection.

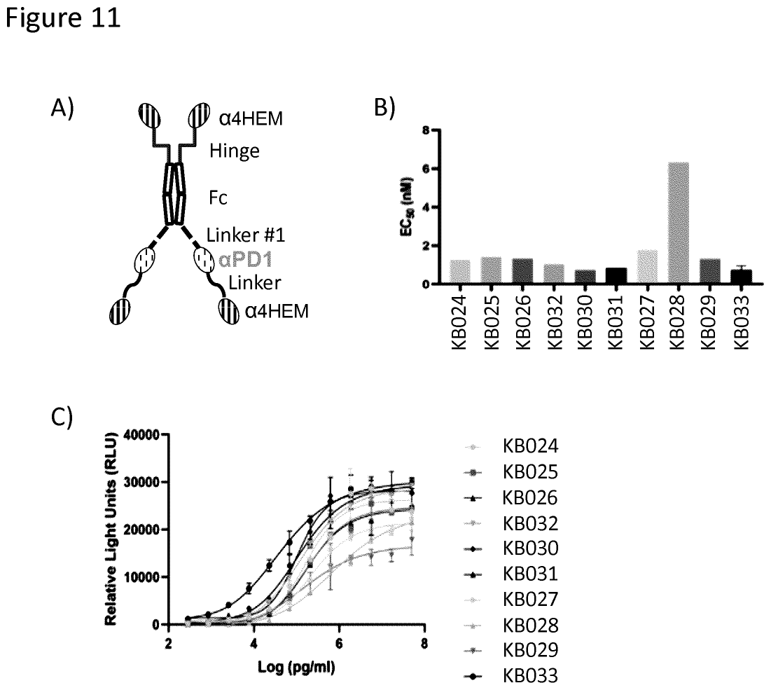

In a first set of experiments, the linker immediately adjacent to the C-terminal part of Fc was selected amongst the linker sequences set forth in SEQ ID NO:3, SEQ ID NO:5, SEQ ID NO:6, SEQ ID NO:8, SEQ ID NO:9, SEQ ID NO:10, SEQ ID NO:12, SEQ ID NO:13 and SEQ ID NO:14 (FIG. 11A).

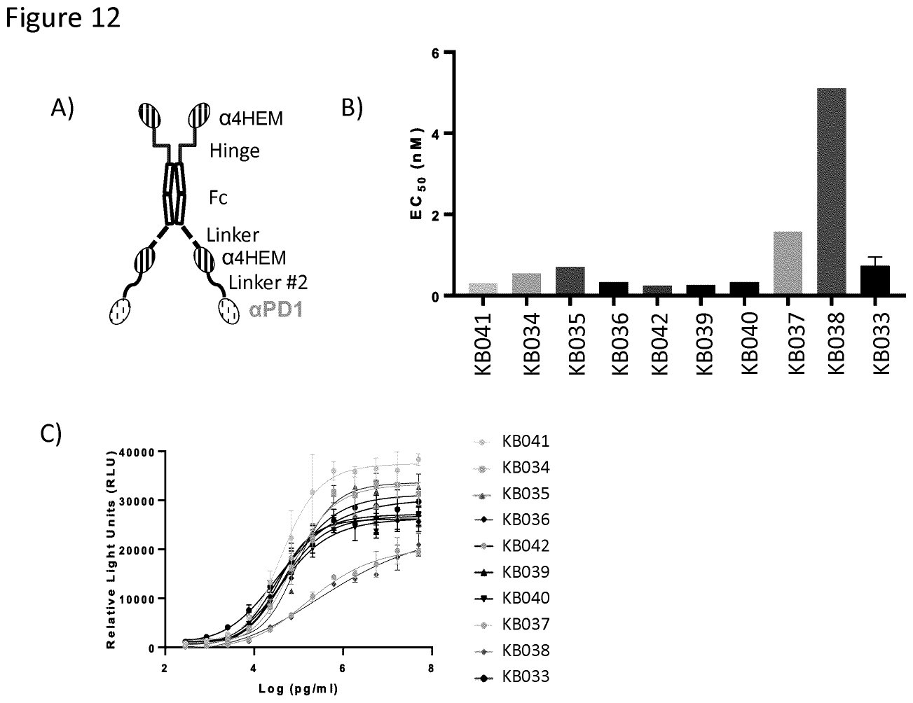

In a second set of experiments the linker between the two C-terminal VHHs was selected amongst the linker sequences set forth in SEQ ID NO:3, SEQ ID NO:4, SEQ ID NO:6, SEQ ID NO:7 (n=1), SEQ ID NO:8, SEQ ID NO:9, SEQ ID NO:10, SEQ ID NO:13 and SEQ ID NO:14 (FIG. 12A).

Results of these experiments presented in FIGS. 11B and 11C as well as in FIGS. 12B and 12C show that the binding of the anti-PD-1 VHH to its target is affected by the linker type and length in the protein dimers. In this context, it appears that rigid and flexible linkers bind with an affinity similar to that of the positive control. The presence of the helical linker having the sequence set forth in SEQ ID NO:13 at a position immediately adjacent to the C-terminal part of Fc have greater impact on the binding of the protein dimers. The presence of the helical linker having the sequence set forth in SEQ ID NO:14 and to a lower extent the linker having the sequence set forth in SEQ ID NO:13 between the two C-terminal VHHs also have an adverse impact on the binding of the protein dimers. In this context, rigid linkers appear preferable for joining a VHH at the C-terminal of the dimerization domain and between the two VHHs located at the C-terminus of the dimerization domain.

### Results for Tumor-Specific Polypeptides and Protein Dimers

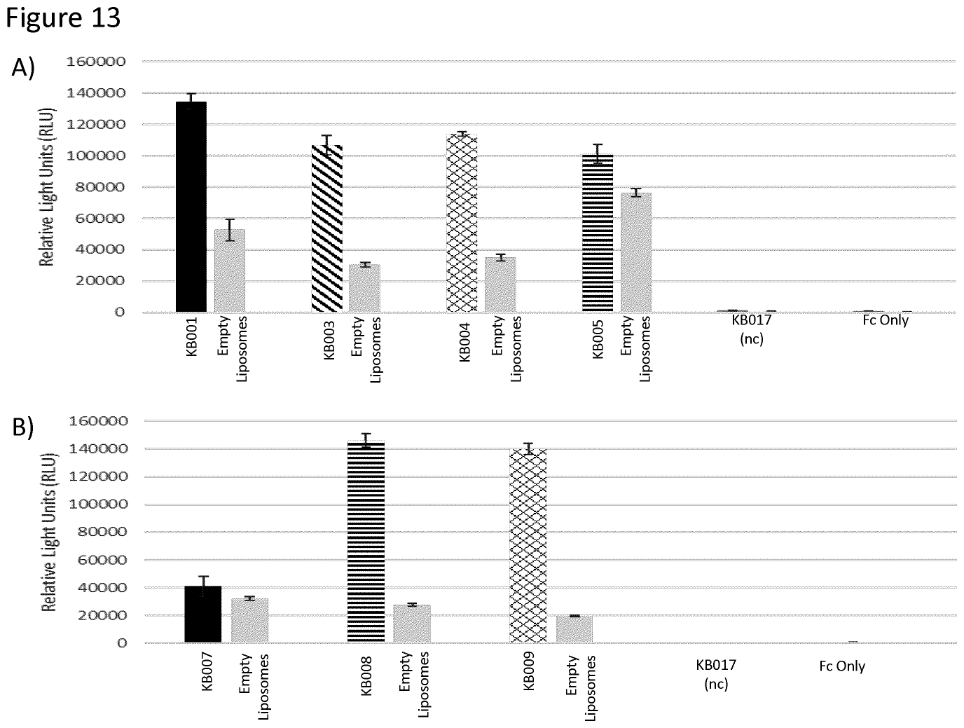

Exemplary results of experiments carried out with tumor-specific polypeptides and related protein dimers are presented in FIGS. 13 to 20.

### Binding Assays

The binding of homodimers made from polypeptides containing distinct anti-DRD2 VHH moieties was determined by Proteoliposome ELISA as described herein. Proteoliposomes containing the DRD2 protein or empty liposomes were used as target and homodimers made from the KB001, KB003, KB004, KB005 or KB017 (negative control) polypeptides were tested at a concentration 1 uM.

Results presented in FIG. 13A show that homodimers made from the KB001, KB003, KB004 and KB005 polypeptide selectively bind to DRD2.

Similar experiments were carried out with homodimers made from polypeptides containing distinct anti-DRD1 VHH moieties (KB035, KB008, KB009). The results of this experiment are presented in FIG. 13B and show that at least homodimers made from the KB008 and KB009 polypeptide selectively bind to DRD1.

ELISA experiments carried out with homodimers containing anti-CD36 VHH moieties show that at least homodimers made from the KB014 polypeptide bind efficiently to recombinant human CD36 (data not shown).

Variability in the binding of the homodimers to their target is likely due to variation in the binding affinity or avidity of the tumor-specific VHHs to their epitopes.

### In Vitro Cell Viability and Cytotoxicity

Homodimers made from polypeptides containing DRD2 targeting moieties were tested for their ability to decrease the viability of NCI-H510A or NCI-H69 in vitro using the CellTiter-Fluor™ Cell Viability Assay (Promega) according to manufacturer's instructions. Briefly, the viability of NCI-H510A or NCI-H69 cells incubated with homodimers made from the KB001, KB003, KB004 and KB005 polypeptides (concentration of 1,000 ng/ml) was compared with the negative control homodimers made from the KB018 polypeptide, or to the PBS vehicle control.

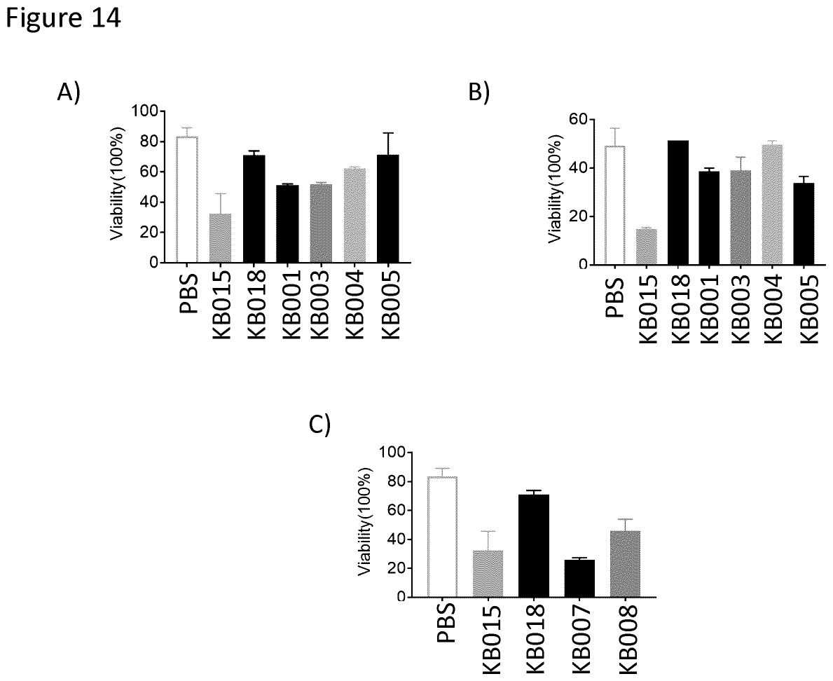

Results are presented in FIG. 14A and FIG. 14B and show that DRD2-specific homodimers decrease viability of the NCI-H510A (FIG. 14A) and NCI-H69 (FIG. 14B) lung cancer cell lines.

Similar experiments were conducted to determine the viability of NCI-H510A cells with the homodimers made from polypeptides containing distinct DRD1 targeting moieties (KB035 and KB008). Results of this experiment are presented in FIG. 14C and show that homodimers made from the KB007 and KB008 polypeptides efficiently decrease the viability of the NCI-H510A lung cancer cells.

Homodimers made from polypeptides containing CD36 targeting moieties were tested for in vitro cytotoxicity using the tumor cell line OCI-AML3 as indicated above. The cells were treated with homodimers made from the KB017, KB019, KB012, or KB013 polypeptides at concentration of 0 nM, 0.0667 nM, 0.667 nM, and 6.67 nM. Cells were incubated at 4° C. for 10 minutes on ice with 7AAD staining solution. After incubation, the cells were stained, and the percentage of dead cells was compared to that of viable cells.

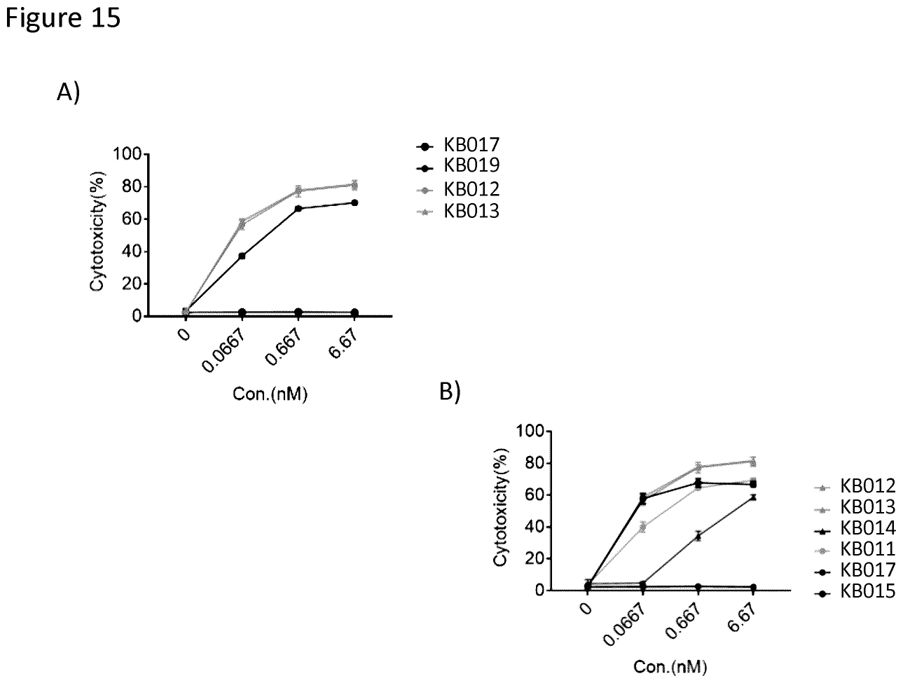

Results presented in FIG. 15A show that addition of VHH targeting cancer specific antigen CD36 enhances the functional cytotoxic activity of the homodimers.

A separate experiment conducted with homodimers made from the KB014 and KB011 polypeptide show that these constructs also efficiently induce OCI-AML cytotoxicity (FIG. 15B).

### Example 4: In Vivo Testing

### Established Tumor Model

Forty female NOG mice (Taconics), aged between 6-8 weeks were injected subcutaneously with 2 million human OCI-AML3 leukemia tumor cells and with 100 μL human PBMCs injected intraperitoneally. Additional PBMCs were injected when the tumor reached 100-200 mm³. Treatment was started 2-4 days after PBMC injection. The mice were divided into 4 treatment groups of 10 animals each of the same average tumor size. Treatment consisted of an antibody dose of approximately 30 mg/kg of multivalent protein dimers or a vehicle (PBS) control, twice per week for a duration of 3 weeks.

### Tumor Prevention Model

Forty female NOG mice (Taconics), aged of 6-8 weeks were injected subcutaneously with 2 million human OCI-AML3 Leukemia tumor cells mixed (1:1) with 7 million human PBMC. The mice were divided into 4 treatment groups of 10 animals each. Treatment consisted of an antibody dose of approximately 30 mg/kg of multivalent protein dimers or a vehicle (PBS) control, twice per week for a duration of 3 weeks.

### Results for Polypeptides and Protein Dimers

In a first set of experiments, the anti-tumor effect of homodimers made from the KB019 or KB015 polypeptide was compared in a preventative in vivo tumor model using the human OCI-AML3 leukemia tumor cells. Briefly, female NOG mice (Taconics) aged between −6-8 weeks were injected subcutaneously with 2 million OCI-AML3 tumor cells mixed with 7 million human PBMCs. Mice were divided into treatment groups consisting of 10 animals each. Treatment consisted of a dose of 28 mg/kg of the homodimers (made from the KB017, KB019, KB015 polypeptides), or a vehicle (PBS) control, twice per week for a duration of 3 weeks during which tumor size was measured, and tumor volume calculated.

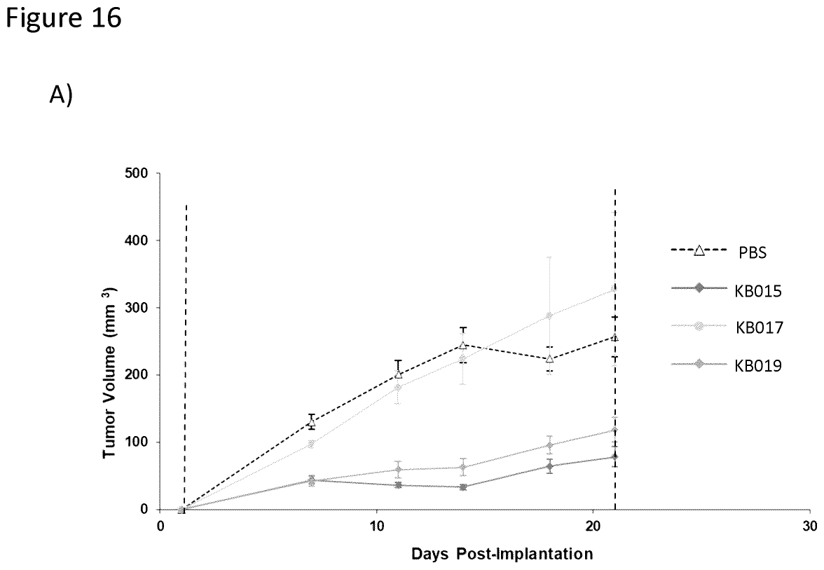

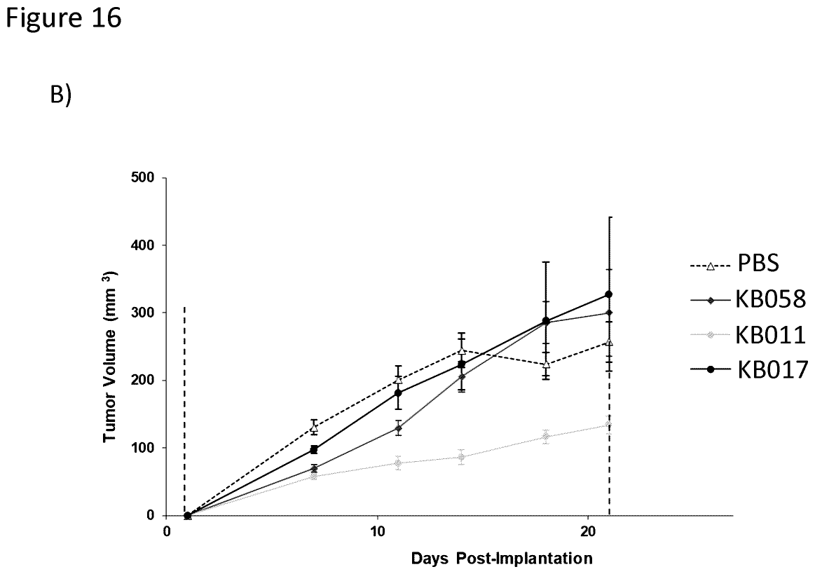

Results presented in FIG. 16A show that VHHs targeting CD3 and PD1 confer in vivo functional activity to homodimers made from the KB019 polypeptide and that addition of the tumor targeting single domain antibody against PDL1 enhances functional activity of the molecule (see results for homodimers made from the KB015 polypeptide). Similar results were also obtained in another immunodeficient mice model (using NCG mice (Charles River) data not shown).

### Results for Tumor-Specific Polypeptides and Protein Dimers

The anti-tumor effect of homodimers made from the KB011 polypeptide was compared in a preventative in vivo tumor model using the human OCI-AML3 leukemia tumor cells with that of homodimers made from the KB017 polypeptide and with that of a construct containing the same anti-CD36 VHH but in sdAb form, VHH-Fc (KB058). Briefly, female NOG mice (Taconics) aged between 4-6 weeks were injected subcutaneously with 2 million OCI-AML3 tumor cells mixed with 7 million human PBMC. The mice were divided into treatment groups consisting of 10 animals each. Treatment consisted of a dose of 28 mg/kg of the homodimers (made from the KB011, KB015, KB058 polypeptide), or a vehicle (PBS) control, twice per week for a duration of 3 weeks after which tumor size was measured, and tumor volume calculated.

Results presented in FIG. 16B show that homodimers made from the KB011 polypeptide are more effective in vivo than the VHH-Fc counterpart (KB058). The efficiency of the homodimers was also demonstrated in the NCG (Charles River) immunodeficient mice model (data not shown).

### Tumor Model in CB-17 Fox Chase SCID Mice

Tumor cells (8 million cells/mouse for NCI-H510A) in DPBS were injected subcutaneously (s.c.) into the right flank of mice. One day after the cell inoculations, mice inoculated with each cell line were randomly divided into 2 experimental groups (10 or 5 mice/group), and each group of mice received 16 mg/kg i.p. either negative control or KB120, twice weekly, for a total of eight weeks. Tumor volumes were measured using vernier calipers and the mice were weighed one or two times weekly. Tumor volume was calculated using the formula: 1/2 (Length×Width²). For calculation of percentage tumor growth inhibition (TGI), KB120 treated group was compared with its respective negative control. TGI was calculated by the following formula:

$$T G I [ \% ] = 100 - \frac{mean ( {TV ( treated )}_{day z} - {TV ( treated )}_{day x} )}{mean ( {TV ( resp \cdot control )}_{day z} - {TV ( resp \cdot control )}_{day x} )} \times 100$$

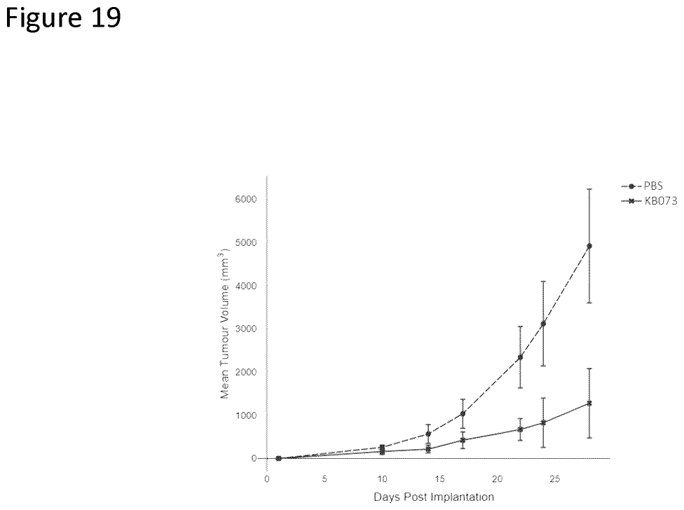

TV day z represents the tumor volume of an individual animal at a defined study day (day z) and TV day x represents the tumor volume of an individual animal at the staging day (day x). DRD2 trispecific protein complex KB073 (specific for DRD2, PD1 and CD3) was tested in a cancer xenograft model. NCG mice were inoculated with NCI-H82 cells with human PBMC and NCI-H82 cells which were co-engrafted at a ratio of 1:5 (FIG. 19).

Statistical tests were performed by a student t-test (two-tailed).

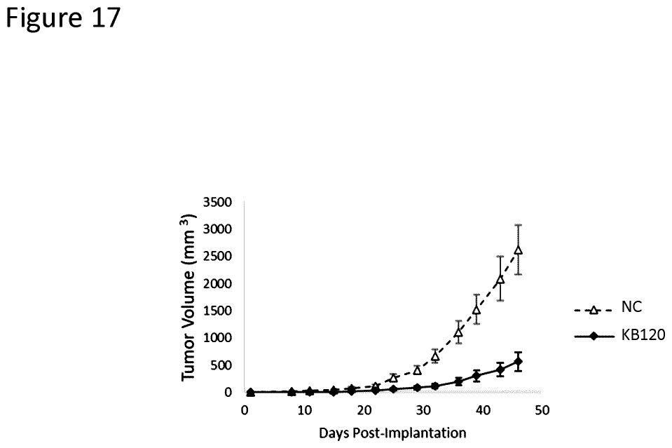

Results presented in FIG. 17 show that the anti-DRD2 VHH is functional as a sdAb (anti-DRD2 antigen binding domain fused at the N-terminus of human hinge followed by Fc at the C-terminus). The KB120 construct reduces tumor volume compared to negative control in NCI-H510A model as well as in other models.

Polypeptide complexes formed by the assembly of polypeptide chains comprising VHHs having specificity for DRD2, PD1 and/or CD3 are tested for in vitro and in vivo activity as outlined herein. Results of FIG. 19 show that an exemplary DRD2 trispecific protein complex has anti-tumor effect in the NCI-H82 SCLC human PBMC co-engraftment model. The anti-tumor effect of the protein complex is also increased when administered in combination with chemotherapy such as cisplatin (data not shown).

### PD-1/PD-L1 Blockade Bioassay

In a first set of experiments, the binding of protein complexes that comprise an anti-PD-1 VHH on recombinant human PD-1 was assessed by ELISA.

Briefly, recombinant proteins were coated on 96-well plates. Plates were covered and left at 4° C. overnight. The next day, plates were washed once with PBS and blocked with blocking buffer for 1 hour at room temperature. Antibodies were tested at the indicated concentration, diluted in the blocking buffer, and incubated for 1 hour at 37° C. After incubation, plates were washed three times with washing buffer. Plates were incubated with anti-human-Fc-HRP diluted at 1:5000 for 1 hour at room temperature, then washed three times with PBS-T washing buffer. The signal was developed with SuperSignal™ ELISA Pico Chemiluminescent Substrate. The plates were read on a SpectraMax™ i3x Multi-Mode Microplate Reader (Molecular Devices).

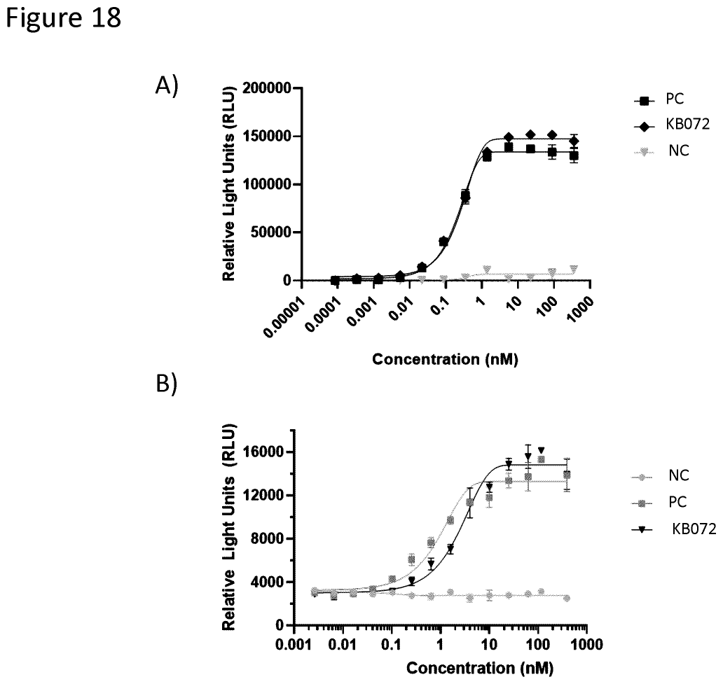

Results of this experiment shows that the KB072 protein complex comprising an anti-PD-1 VHH bind to recombinant human PD-1 as efficiently as the positive control (FIG. 18A).

The blockade activity of protein complexes that comprise an anti-PD-1 VHH was assessed using a PD-1/PD-L1 blockade assay within a luminescent NFAT-RE reporter system and compared to a positive control (FIG. 18B). Briefly, PD-L1 aAPC/CHO-K1 (target) cells were thawed and seeded into 96-well plate at the recommended density and allowed to adhere to the plate overnight. The following day, protein complexes were diluted to 350 nM in assay buffer (Ham's F12 media with 10% low IgG FBS), and eight 2.5x serial dilutions were conducted. Media from the 96-well plate was decanted, and 40 μl of diluted polypeptides or protein complex and 40 μl Jurkat (effector) cells were added to the plate. Plates were incubated at 37° C. for 6 hours. Bio-Glo luciferase assay buffer and substrate were combined, and 80 μl of the solution was transferred to each well. The plate was incubated at room temperature for 5 minutes, and the luminescence was measured using a plate reader.

Results of this experiment indicate that the binding of the KB072 protein complex shows similar activity to a positive control (FIG. 18B). The anti-PD-1 VHH remains functional even when located between the “Fc-linker” component and the “linker-VHH” component. The anti-PD-1 module is therefore functional even if located at the C-terminus of Fc. Binding of the protein complex to cells expressing PD-1 was also observed by FACS assay (data not shown). Protein complexes comprising an anti-PD-1 VHH also increases PBMC-mediated cytotoxicity (data not shown).

Accumulation of radiolabeled protein complexes in tumor tissues was observed in DRD2-positive NCI-H69 and NCI-H82 xenograft models of human small cell lung cancer (data not shown).

### Example 5: Heterodimers

### Heterodimer Design

In order to generate heterodimers, the Applicant introduced a number of mutations in the CH3 domain of the Fc portion so as to remove electrostatic interactions or to introduce repulsive charges.

Briefly, DNA constructs containing mutations at positions 356, 370 and 399 (in accordance with EU numbering system) were generated. In some of these constructs (Chain A), the glutamic acid (E) at position 356 was changed for glutamine (Q), the lysine (K) at position 370 was changed for glutamic acid (E) and the aspartic acid (D) at position 399 was changed for asparagine (N). These constructs contain a His tag that helps in the detection of the proteins which is not necessary to its function. Other DNA constructs (Chain B) containing mutations at positions 357, 399 and 439 (in accordance with EU numbering system) were generated. More particularly, in some of these constructs, the glutamic acid (E) at position 357 was changed for glutamine (Q), the aspartic acid (D) at position 399 was changed for asparagine (N) and the lysine (K) at position 439 was modified for glutamic acid (E).

Polypeptides comprising Chain A or Chain B mutations may assemble into homodimers when expressed in cells in the absence of the other chain.

TABLE 3
Exemplary POP polypeptides containing mutated dimerization domain

| | | Module 1 | Module 2 | Module 3 | Module 4 | Module 5 | Module 6 | Module 7 |
|---|---|---|---|---|---|---|---|---|
| Code Name | Specificity | VHH 1 | Linker 1 | Fc | Linker 2 | VHH 2 | Linker 3 | VHH 3 |
| KB047 | PDL1, | α-PDL1, | SEQ ID | SEQ ID | SEQ ID | α-CD3 | SEQ ID | α-PD1 |
| (Chain B) | CD3, PD1 | SEQ ID | NO: 1 | NO: 19 | NO: 2 | SEQ ID | NO: 4 | SEQ ID |
| | | NO: 24 | | | | NO: 20 | | NO: 21 |
| KB068 | PDL1, | α-PDL1, | SEQ ID | SEQ ID | SEQ ID | α-CD3 | SEQ ID | α-PD1 |
| (Chain B) | CD3, PD1 | SEQ ID | NO: 1 | NO: 19 | NO: 9 | SEQ ID | NO: 4 | SEQ ID |
| | | NO: 24 | | | | NO: 20 | | NO: 21 |
| KB048 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 19 | NO: 2 | NO: 22 | NO: 4 | NO: 22 |
| | HEWL | | | | | | | |
| KB050 | 4HEM, | SEQ ID | SEQ ID | SEQ ID | SEQ ID | SEQ ID | SEQ ID | SEQ ID |
| (Chain A) | 4HEM, | NO: 23 | NO: 1 | NO: 18 | NO: 5 | NO: 23 | NO: 4 | NO: 23 |
| | 4HEM | | | | | | | |
| KB049 | 4HEM, | SEQ ID | SEQ ID | SEQ ID | SEQ ID | SEQ ID | — | — |
| (Chain B) | 4HEM | NO: 23 | NO: 1 | NO: 19 | NO: 5 | NO: 23 | | |

TABLE 4
Exemplary tumor-specific polypeptides containing mutated dimerization domain

| | | Module 1 | Module 2 | Module 3 | Module 4 | Module 5 | Module 6 | Module 7 |
|---|---|---|---|---|---|---|---|---|
| Code Name | Specificity | VHH 1 | Linker 1 | Fc | Linker 2 | VHH 2 | Linker 3 | VHH 3 |
| KB002 | DRD2, | α- | SEQ ID | SEQ ID | SEQ ID | α-PD1 | SEQ ID | α-CD3 |
| (Chain B) | PD1, CD3 | DRD2.1 | NO: 1 | NO: 19 | NO: 9 | SEQ ID | NO: 4 | SEQ ID |
| | | | | | | NO: 21 | | NO: 20 |
| KB069 | DRD2, | α- | SEQ ID | SEQ ID | SEQ ID | α-CD3 | SEQ ID | α-PD1 |
| (Chain B) | CD3, PD1 | DRD2.1 | NO: 1 | NO: 19 | NO: 9 | SEQ ID | NO: 4 | SEQ ID |
| | | | | | | NO: 20 | | NO: 21 |
| KB051 | DRD1, | α- | SEQ ID | SEQ ID | SEQ ID | α-CD3 | — | — |
| (Chain A) | CD3 | DRD1.4 | NO: 1 | NO: 18 | NO: 14 | SEQ ID | | |
| | | | | | | NO: 20 | | |
| KB051 | DRD1, | α- | SEQ ID | SEQ ID | SEQ ID | α-CD3 | SEQ ID | α-PD1 |
| (Chain B) | CD3, PD1 | DRD1.3 | NO: 1 | NO: 19 | NO: 2 | SEQ ID | NO: 4 | SEQ ID |
| | | | | | | NO: 20 | | NO: 21 |
| KB052 | DRD1, | α- | SEQ ID | SEQ ID | SEQ ID | α-CD3 | — | — |
| (Chain A) | CD3 | DRD1.3 | NO: 1 | NO: 18 | NO: 14 | SEQ ID | | |
| | | | | | | NO: 20 | | |
| KB052 | DRD1, | α- | SEQ ID | SEQ ID | SEQ ID | α-CD3 | SEQ ID | α-PD1 |
| (Chain B) | CD3, PD1 | DRD1.1 | NO: 1 | NO: 19 | NO: 2 | SEQ ID | NO: 4 | SEQ ID |
| | | | | | | NO: 20 | | NO: 21 |
| KB053 | DRD1, | α- | SEQ ID | SEQ ID | SEQ ID | α-CD3 | — | — |
| (Chain A) | CD3 | DRD1.1 | NO: 1 | NO: 18 | NO: 14 | SEQ ID | | |
| | | | | | | NO: 20 | | |
| KB053 | DRD1, | α- | SEQ ID | SEQ ID | SEQ ID | α-CD3 | SEQ ID | α-PD1 |
| (Chain B) | CD3, PD1 | DRD1.3 | NO: 1 | NO19 | NO: 2 | SEQ ID | NO: 4 | SEQ ID |
| | | | | | | NO: 20 | | NO: 21 |
| KB054 | DRD2, | α- | SEQ ID | SEQ ID | SEQ ID | α-CD3 | — | — |
| (Chain A) | CD3 | DRD2.1 | NO: 1 | NO: 18 | NO: 14 | SEQ ID | | |
| | | | | | | NO: 20 | | |
| KB054 | DRD2, | α- | SEQ ID | SEQ ID | SEQ ID | α-CD3 | SEQ ID | α-PD1 |
| (Chain B) | CD3, PD1 | DRD2.4 | NO: 1 | NO19 | NO: 2 | SEQ ID | NO: 4 | SEQ ID |
| | | | | | | NO: 20 | | NO: 21 |
| KB055 | DRD2, | α- | SEQ ID | SEQ ID | SEQ ID | SEQ ID | SEQ ID | SEQ ID |
| (Chain B) | HEWL, | DRD2.1 | NO: 1 | NO19 | NO: 2 | NO: 22 | NO: 4 | NO: 22 |
| | HEWL | | | | | | | |
| KB056 | DRD2, | α- | SEQ ID | SEQ ID | SEQ ID | SEQ ID | SEQ ID | α-CD3 |
| (Chain B) | HEWL, | DRD2.1 | NO: 1 | NO19 | NO: 9 | NO: 22 | NO: 4 | SEQ ID |
| | CD3 | | | | | | | NO: 20 |

In order to test if CH3 mutations affect the cytotoxicity of the molecule, a variant of the KB015 polypeptide containing mutations at positions 357, 399 and 439 (i.e., the KB047 polypeptide) was made, transfected into cells and the protein dimers thus generated were tested for their cytotoxicity in an in vitro assay as described above.

Briefly, target OCI-AML3 tumor cells were pre-labelled with Cell Trace Violet then treated with of human PBMC effector cells (effector to target ratio 5:1) in the presence of protein dimers made from the KB015 or KB047 polypeptide, or with the negative control dimers made from the KB018 or KB048 polypeptide at final concentration of 0 nM, 0.007 nM, 0.07 nM, 0.7 nM, and 7 nM. After incubation, the dead cells were stained with 7-AAD, and the cytotoxicity was calculated as the percentage of dead cells was compared to the number target OCI-AML3 tumor cells.

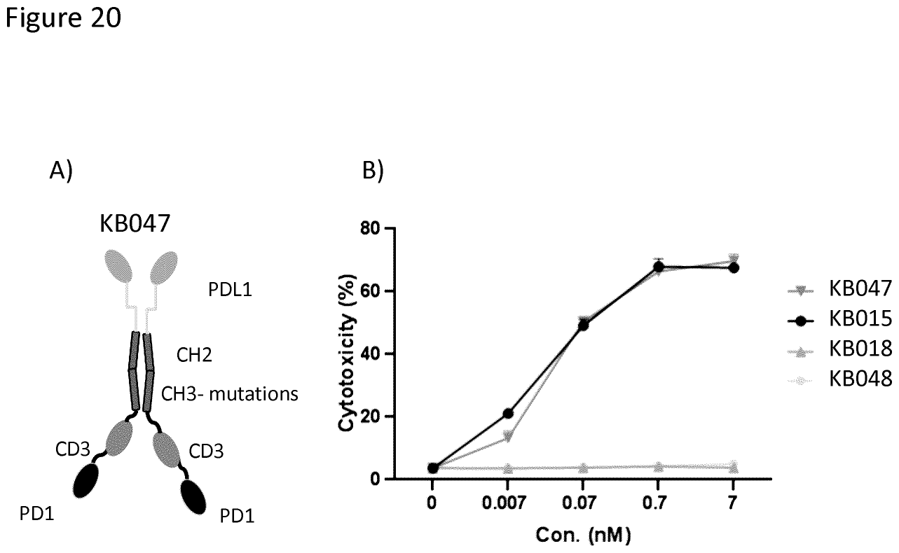

Results of this experiment are presented in FIG. 20B and show that the cytotoxic effect of dimers made from the KB047 polypeptide is similar to that of the dimers made from the KB015 polypeptide. As such, these mutations do not appear to negatively affect the cytotoxicity of the molecule.

### Formation of Heterodimers

A DNA construct comprising three anti-4HEM VHHs and a Fc region containing mutations D399N, K439E, E357Q (in accordance with EU numbering system) was generated (the KB049 polypeptide).

Another DNA construct containing two anti-4HEM VHHs and a Fc region containing mutations D399N, K370E and E356Q (in accordance with EU numbering system) was also generated (the KB050 polypeptide).

Polypeptides were expressed using the ExpiCHO™ Expression System (Thermo Fisher, Cat. no. A29133) as described above.

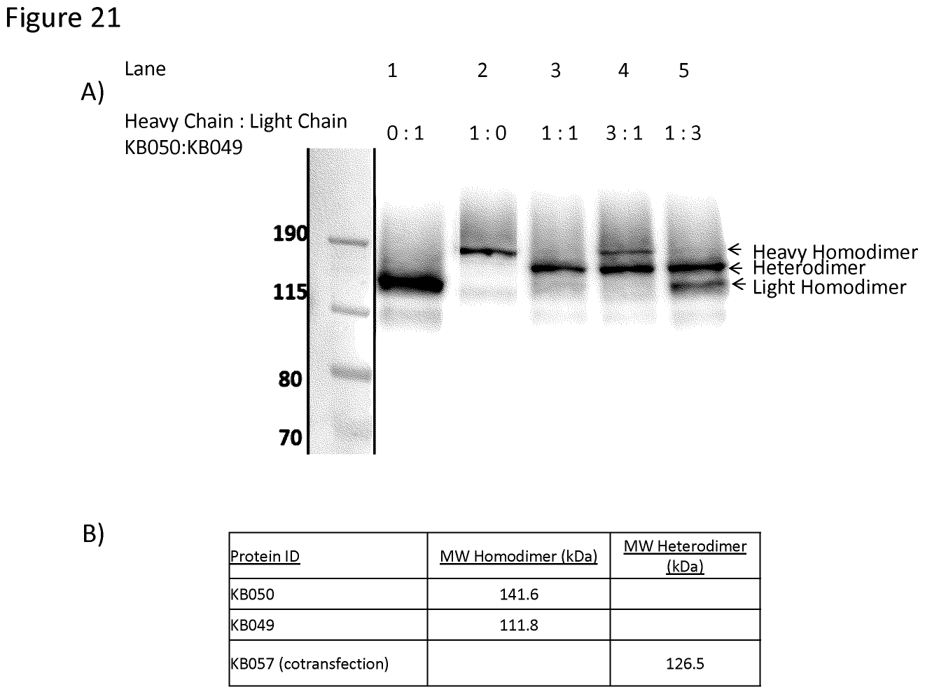

Briefly, cells were transfected with either a plasmid encoding the lighter chain (the KB049 polypeptide), heavier chain (the KB050 polypeptide) or co-transfected with both plasmids (identified as KB057 in FIG. 21B) at ratios of 1:1, 3:1, and 1:3. Eight days after transfection supernatants were clarified, filter sterilized and stored at 4° C. or frozen for later analysis. The production of heterodimers was analyzed by Western blot and detected using the Penta·His antibody.

Expression of only the heavier chain KB050 polypeptide resulted in the formation of heavy homodimers, while expression of only the lighter chain KB049 resulted in only light homodimer production. Results of this experiment are presented in FIG. 21 (non-reducing conditions) and show that heterodimers are efficiently formed, with optimal heterodimer formation occurring when DNA ratio is at 1 to 1. Co-expression of both heavier and lighter chains (KB057) resulted in the successful production of heterodimers (FIG. 21).

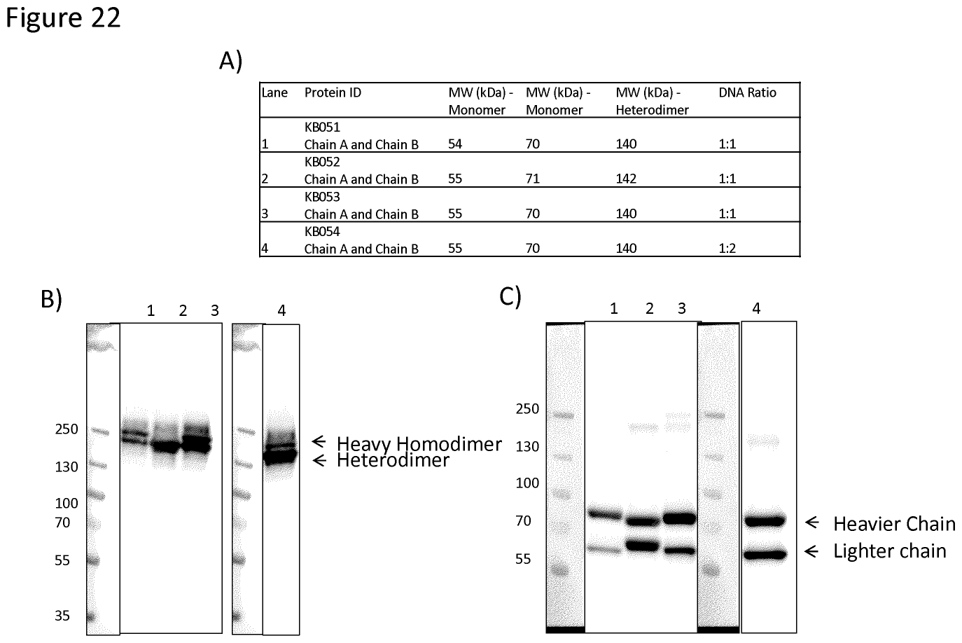

Similar experiments were conducted to examine the effect of changing the VHH domains where several variants were tested that have alternate antigen binding domains by co-expressing Chain A and Chain B of KB051, KB052, KB053 at DNA ratio of 1:1 or KB054 at DNA ratio of 1:2 (FIG. 22A). Results of these experiments presented in FIG. 22 show that heterodimers can be efficiently formed from these constructs which encode proteins with a variety of VHH domains. (FIG. 22B—non reduced conditions, FIG. 22C—reduced conditions).

Additional mutated CH3 domains were generated and mutant polypeptides were tested for their ability to assemble into heterodimers. Exemplary polypeptide chains are presented in Table 5.

| CH3 | | Module 1 | Module 2 | Module 3 | Module 4 | Module 5 | Module 6 | Module 7 |
|---|---|---|---|---|---|---|---|---|
| Domain ID | Specificity | VHH1 | Linker 1 | Fc | Linker 2 | VHH2 | Linker 3 | VHH3 |
| KB079 | HEWL, | SEQ ID | SEQ ID | SEQ ID | SEQ ID | SEQ ID | | |
| (Chain A) | HEWL, | NO: 22 | NO: 1 | NO: 18 | NO: 14 | NO: 22 | | |
| KB080 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 19 | NO: 9 | NO: 22 | NO: 4 | NO: 22 |
| | HEWL | | | | | | | |
| KB081 | HEWL, | SEQ ID | SEQ ID | SEQ ID | SEQ ID | SEQ ID | | |
| (Chain A) | HEWL | NO: 22 | NO: 1 | NO: 53 | NO: 9 | NO: 22 | | |
| KB082 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 54 | NO: 9 | NO: 22 | NO: 4 | NO: 22 |
| | HEWL | | | | | | | |
| KB083 | DRD2, | α- | SEQ ID | SEQ ID | SEQ ID | SEQ ID | | |
| (Chain A) | PD1 | DRD2.1 | NO: 1 | NO: 55 | NO: 9 | NO: 21 | | |
| KB084 | DRD2, | α- | SEQ ID | SEQ ID | SEQ ID | SEQ ID | SEQ ID | SEQ ID |
| (Chain B) | PD1, | DRD2.1 | NO: 1 | NO: 56 | NO: 9 | NO: 21 | NO: 4 | NO: 20 |
| | CD3 | | | | | | | |
| KB085 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 57 | NO: 9 | NO: 22 | NO: 4 | NO: 22 |
| | HEWL | | | | | | | |
| KB086 | HEWL, | SEQ ID | SEQ ID | SEQ ID | SEQ ID | SEQ ID | | |
| (Chain A) | HEWL | NO: 22 | NO: 1 | NO: 58 | NO: 9 | NO: 22 | | |
| KB087 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 59 | NO: 9 | NO: 22 | NO: 4 | NO: 22 |
| | HEWL | | | | | | | |
| KB088 | HEWL, | SEQ ID | SEQ ID | SEQ ID | SEQ ID | SEQ ID | | |
| (Chain A) | HEWL | NO: 22 | NO: 1 | NO: 60 | NO: 9 | NO: 22 | | |
| KB089 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 61 | NO: 9 | NO: 22 | NO: 4 | NO: 22 |
| | HEWL | | | | | | | |
| KB090 | HEWL, | SEQ ID | SEQ ID | SEQ ID | SEQ ID | SEQ ID | | |
| (Chain A) | HEWL | NO: 22 | NO: 1 | NO: 62 | NO: 9 | NO: 22 | | |
| KB091 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 63 | NO: 9 | NO: 22 | NO: 4 | NO: 22 |
| | HEWL | | | | | | | |
| KB092 | HEWL, | SEQ ID | SEQ ID | SEQ ID | SEQ ID | SEQ ID | | |
| (Chain A) | HEWL | NO: 22 | NO: 1 | NO: 64 | NO: 9 | NO: 22 | | |
| KB093 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 65 | NO: 9 | NO: 22 | NO: 4 | NO: 22 |
| | HEWL | | | | | | | |
| KB094 | HEWL, | SEQ ID | SEQ ID | SEQ ID | SEQ ID | SEQ ID | | |
| (Chain A) | HEWL | NO: 22 | NO: 1 | NO: 66 | NO: 9 | NO: 22 | | |
| KB095 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 67 | NO: 9 | NO: 22 | NO: 4 | NO: 22 |
| | HEWL | | | | | | | |
| KB096 | HEWL, | SEQ ID | SEQ ID | SEQ ID | SEQ ID | SEQ ID | | |
| (Chain A) | HEWL | NO: 22 | NO: 1 | NO: 68 | NO: 9 | NO: 22 | | |
| KB097 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 69 | NO: 9 | NO: 22 | NO: 4 | NO: 22 |
| | HEWL | | | | | | | |
| KB098 | HEWL, | SEQ ID | SEQ ID | SEQ ID | SEQ ID | SEQ ID | | |
| (Chain A) | HEWL | NO: 22 | NO: 1 | NO: 70 | NO: 9 | NO: 22 | | |
| KB099 | HEWL, | SEQ ID | SEQ ID | SEQ ID | SEQ ID | SEQ ID | SEQ ID | SEQ ID |
| (Chain B) | HEWL, | NO: 22 | NO: 1 | NO: 71 | NO: 9 | NO: 22 | NO: 4 | NO: 22 |
| | HEWL | | | | | | | |
| KB100 | HEWL, | SEQ ID | SEQ ID | SEQ ID | SEQ ID | SEQ ID | | |
| (Chain A) | HEWL | NO: 22 | NO: 1 | NO: 72 | NO: 9 | NO: 22 | | |
| KB101 | DRD2, | α- | SEQ ID | SEQ ID | SEQ ID | α-PD1 | | |
| (Chain A) | PD1 | DRD2.1 | NO: 1 | NO: 73 | NO: 9 | | | |
| KB102 | DRD2, | α- | SEQ ID | SEQ ID | SEQ ID | α-PD1 | | |
| (Chain A) | PD1 | DRD2.1 | NO: 1 | NO: 74 | NO: 9 | | | |
| KB103 | DRD2, | α- | SEQ ID | SEQ ID | SEQ ID | α-PD1 | | |
| (Chain A) | PD1 | DRD2.1 | NO: 1 | NO: 75 | NO: 9 | | | |
| KB104 | DRD2, | α- | SEQ ID | SEQ ID | SEQ ID | α-PD1 | | |
| (Chain A) | PD1 | DRD2.1 | NO: 1 | NO: 76 | NO: 9 | | | |
| KB105 | DRD2, | α- | SEQ ID | SEQ ID | SEQ ID | α-PD1 | | |
| (Chain A) | PD1 | DRD2.1 | NO: 1 | NO: 77 | NO: 9 | | | |
| KB106 | DRD2, | α- | SEQ ID | SEQ ID | SEQ ID | α-PD1 | | |
| (Chain A) | PD1 | DRD2.1 | NO: 1 | NO: 78 | NO: 9 | | | |
| KB107 | DRD2, | α- | SEQ ID | SEQ ID | SEQ ID | α-PD1 | | |
| (Chain A) | PD1 | DRD2.1 | NO: 1 | NO: 79 | NO: 9 | | | |
| KB108 | DRD2, | α- | SEQ ID | SEQ ID | SEQ ID | α-PD1 | | |
| (Chain A) | PD1 | DRD2.1 | NO: 1 | NO: 80 | NO: 9 | | | |
| KB109 | DRD2, | α- | SEQ ID | SEQ ID | SEQ ID | α-PD1 | | |
| (Chain A) | PD1 | DRD2.1 | NO: 1 | NO: 81 | NO: 9 | | | |
| KB110 | DRD2, | α- | SEQ ID | SEQ ID | SEQ ID | α-PD1 | | |
| (Chain A) | PD1 | DRD2.1 | NO: 1 | NO: 82 | NO: 9 | | | |
| KB111 | DRD2, | α- | SEQ ID | SEQ ID | SEQ ID | α-PD1 | | |
| (Chain A) | PD1 | DRD2.1 | NO: 1 | NO: 83 | NO: 9 | | | |
| KB112 | DRD2, | α- | SEQ ID | SEQ ID | SEQ ID | α-PD1 | | |
| (Chain A) | PD1 | DRD2.1 | NO: 1 | NO: 84 | NO: 9 | | | |
| KB113 | DRD2, | α- | SEQ ID | SEQ ID | SEQ ID | α-PD1 | SEQ ID | SEQ ID |
| (Chain B) | PD1, | DRD2.1 | NO: 1 | NO: 85 | NO: 9 | | NO: 4 | NO: 20 |
| | CD3 | | | | | | | |
| KB114 | DRD2, | α- | SEQ ID | SEQ ID | SEQ ID | α-PD1 | SEQ ID | SEQ ID |
| (Chain B) | PD1, | DRD2.1 | NO: 1 | NO: 86 | NO: 9 | | NO: 4 | NO: 20 |
| | CD3 | | | | | | | |
| KB115 | DRD2, | α- | SEQ ID | SEQ ID | SEQ ID | α-PD1 | SEQ ID | SEQ ID |
| (Chain B) | PD1, | DRD2.1 | NO: 1 | NO: 87 | NO: 9 | | NO: 4 | NO: 20 |
| | CD3 | | | | | | | |
| KB116 | DRD2, | α- | SEQ ID | SEQ ID | SEQ ID | α-PD1 | SEQ ID | SEQ ID |
| (Chain B) | PD1, | DRD2.1 | NO: 1 | NO: 88 | NO: 9 | | NO: 4 | NO: 20 |
| | CD3 | | | | | | | |
| KB117 | DRD2, | α- | SEQ ID | SEQ ID | SEQ ID | α-PD1 | SEQ ID | SEQ ID |
| (Chain B) | PD1, | DRD2.1 | NO: 1 | NO: 89 | NO: 9 | | NO: 4 | NO: 20 |
| | CD3 | | | | | | | |
| KB118 | DRD2, | α- | SEQ ID | SEQ ID | SEQ ID | α-PD1 | SEQ ID | SEQ ID |
| (Chain B) | PD1, | DRD2.1 | NO: 1 | NO: 90 | NO: 9 | | NO: 4 | NO: 20 |
| | CD3 | | | | | | | |
| KB119 | DRD2, | α- | SEQ ID | SEQ ID | SEQ ID | α-PD1 | SEQ ID | SEQ ID |
| (Chain B) | PD1, | DRD2.1 | NO: 1 | NO: 91 | NO: 9 | | NO: 4 | NO: 20 |
| | CD3 | | | | | | | |

Vectors expressing Chain A and Chain B pairs selected from Table 5 below were co-expressed at different ratio, and the formation of heterodimers was assessed as described above. Mutations that disfavor homodimers formation and/or favor heterodimers formation are selected for generating multivalent and/or multispecific protein complexes.

The following pairs of vectors were particularly tested.
A vector expressing a polypeptide comprising the mutated CH3 domain set forth in SEQ ID NO:53 was co-transfected with a vector expressing a polypeptide comprising the mutated CH3 domain set forth in SEQ ID NO: 54.
A vector expressing a polypeptide comprising the mutated CH3 domain set forth in SEQ ID NO:55 was co-transfected with a vector expressing a polypeptide comprising the mutated CH3 domain set forth in SEQ ID NO: 56.
A vector expressing a polypeptide comprising the mutated CH3 domain set forth in SEQ ID NO:57 was co-transfected with a vector expressing a polypeptide comprising the mutated CH3 domain set forth in SEQ ID NO: 58.
A vector expressing a polypeptide comprising the mutated CH3 domain set forth in SEQ ID NO:59 was co-transfected with a vector expressing a polypeptide comprising the mutated CH3 domain set forth in SEQ ID NO: 60.
A vector expressing a polypeptide comprising the mutated CH3 domain set forth in SEQ ID NO:61 was co-transfected with a vector expressing a polypeptide comprising the mutated CH3 domain set forth in SEQ ID NO: 62.
A vector expressing a polypeptide comprising the mutated CH3 domain set forth in SEQ ID NO:63 was co-transfected with a vector expressing a polypeptide comprising the mutated CH3 domain set forth in SEQ ID NO: 64.
A vector expressing a polypeptide comprising the mutated CH3 domain set forth in SEQ ID NO:65 was co-transfected with a vector expressing a polypeptide comprising the mutated CH3 domain set forth in SEQ ID NO: 66.
A vector expressing a polypeptide comprising the mutated CH3 domain set forth in SEQ ID NO:67 was co-transfected with a vector expressing a polypeptide comprising the mutated CH3 domain set forth in SEQ ID NO: 68.
A vector expressing a polypeptide comprising the mutated CH3 domain set forth in SEQ ID NO:69 was co-transfected with a vector expressing a polypeptide comprising the mutated CH3 domain set forth in SEQ ID NO: 70.
A vector expressing a polypeptide comprising the mutated CH3 domain set forth in SEQ ID NO:73 was co-transfected with a vector expressing a polypeptide comprising the mutated CH3 domain set forth in SEQ ID NO: 85.
A vector expressing a polypeptide comprising the mutated CH3 domain set forth in SEQ ID NO:73 was co-transfected with a vector expressing a polypeptide comprising the mutated CH3 domain set forth in SEQ ID NO: 86.
A vector expressing a polypeptide comprising the mutated CH3 domain set forth in SEQ ID NO:73 was co-transfected with a vector expressing a polypeptide comprising the mutated CH3 domain set forth in SEQ ID NO: 87.
A vector expressing a polypeptide comprising the mutated CH3 domain set forth in SEQ ID NO:74 was co-transfected with a vector expressing a polypeptide comprising the mutated CH3 domain set forth in SEQ ID NO: 88.
A vector expressing a polypeptide comprising the mutated CH3 domain set forth in SEQ ID NO:75 was co-transfected with a vector expressing a polypeptide comprising the mutated CH3 domain set forth in SEQ ID NO: 88.
A vector expressing a polypeptide comprising the mutated CH3 domain set forth in SEQ ID NO:76 was co-transfected with a vector expressing a polypeptide comprising the mutated CH3 domain set forth in SEQ ID NO: 88.
A vector expressing a polypeptide comprising the mutated CH3 domain set forth in SEQ ID NO:77 was co-transfected with a vector expressing a polypeptide comprising the mutated CH3 domain set forth in SEQ ID NO: 88.
A vector expressing a polypeptide comprising the mutated CH3 domain set forth in SEQ ID NO:77 was co-transfected with a vector expressing a polypeptide comprising the mutated CH3 domain set forth in SEQ ID NO: 89.
A vector expressing a polypeptide comprising the mutated CH3 domain set forth in SEQ ID NO:77 was co-transfected with a vector expressing a polypeptide comprising the mutated CH3 domain set forth in SEQ ID NO: 90.
A vector expressing a polypeptide comprising the mutated CH3 domain set forth in SEQ ID NO:78 was co-transfected with a vector expressing a polypeptide comprising the mutated CH3 domain set forth in SEQ ID NO: 88.
A vector expressing a polypeptide comprising the mutated CH3 domain set forth in SEQ ID NO:79 was co-transfected with a vector expressing a polypeptide comprising the mutated CH3 domain set forth in SEQ ID NO: 88.
A vector expressing a polypeptide comprising the mutated CH3 domain set forth in SEQ ID NO:80 was co-transfected with a vector expressing a polypeptide comprising the mutated CH3 domain set forth in SEQ ID NO: 90.
A vector expressing a polypeptide comprising the mutated CH3 domain set forth in SEQ ID NO:81 was co-transfected with a vector expressing a polypeptide comprising the mutated CH3 domain set forth in SEQ ID NO: 88.
A vector expressing a polypeptide comprising the mutated CH3 domain set forth in SEQ ID NO:81 was co-transfected with a vector expressing a polypeptide comprising the mutated CH3 domain set forth in SEQ ID NO: 89.
   A vector expressing a polypeptide comprising the mutated CH3 domain set forth in SEQ ID NO:82 was co-transfected with a vector expressing a polypeptide comprising the mutated CH3 domain set forth in SEQ ID NO: 90.
A vector expressing a polypeptide comprising the mutated CH3 domain set forth in SEQ ID NO:83 was co-transfected with a vector expressing a polypeptide comprising the mutated CH3 domain set forth in SEQ ID NO: 88.
A vector expressing a polypeptide comprising the mutated CH3 domain set forth in SEQ ID NO:84 was co-transfected with a vector expressing a polypeptide comprising the mutated CH3 domain set forth in SEQ ID NO: 90.
A vector expressing a polypeptide comprising the mutated CH3 domain set forth in SEQ ID NO:74 was co-transfected with a vector expressing a polypeptide comprising the mutated CH3 domain set forth in SEQ ID NO: 19.
A vector expressing a polypeptide comprising the mutated CH3 domain set forth in SEQ ID NO:55 was co-transfected with a vector expressing a polypeptide comprising the mutated CH3 domain set forth in SEQ ID NO: 19.
A vector expressing a polypeptide comprising the mutated CH3 domain set forth in SEQ ID NO:55 was co-transfected with a vector expressing a polypeptide comprising the mutated CH3 domain set forth in SEQ ID NO: 90.

Results of these experiments indicate that heterodimers are predominantly formed upon co-expression of Chain A and Chain B pairs selected from those comprising mutated CH3 domains set forth in SEQ ID NO:19 and SEQ ID NO:20, set forth in SEQ ID NO:92 and SEQ ID NO:93, in SEQ ID NO:94 and SEQ ID NO:95, set forth in SEQ ID NO:96 and SEQ ID NO:97, set forth in SEQ ID NO:98 and SEQ ID NO:99, set forth in SEQ ID NO: 100 and SEQ ID NO:101, set forth in SEQ ID NO: 102 and SEQ ID NO:95 or set forth in SEQ ID NO:103 and SEQ ID NO:95.

Results of these experiments also show an increased propensity of heterodimers formation when the polypeptide pairs comprise the mutated CH3 domain set forth in SEQ ID NO: 92 and SEQ ID NO:93 or the mutated CH3 domain set forth in SEQ ID NO:94 and SEQ ID NO:95.

The embodiments and examples described herein are illustrative and are not meant to limit the scope of the claims. Variations of the foregoing embodiments, including alternatives, modifications and equivalents, are intended by the inventors to be encompassed by the claims. Citations listed in the present application are incorporated herein by reference.

### REFERENCES

All patents, patent applications and publications referred to throughout the application are incorporated herein by reference.
Angal, S. et al., A Single Amino Acid Substitution Abolishes the Heterogeneity of Chimeric Mouse/human IgG4 Antibody, Mol Immunol 30, 105-108, 1993
Chen X et al., Fusion Protein Linkers: Properties, Design and Functionality, Adv Drug Deliv Rev. 2013; 65(10):1357-1369.
Conrath, K. E. et al., Camel Single-Domain Antibodies as Modular Building Units in Bispecific and Bivalent Antibody Constructs. J. Biol. Chem. 2001, 276, 7346-7350.
Coppieters, K. et al., Formatted Anti-Tumor Necrosis Factor alpha VHH Proteins Derived from Camelids Show Superior Potency and Targeting to Inflamed Joints in a Murine Model of Collagen-induced Arthritis. Arthritis Rheum. 2006, 54, 1856-1866.
Detallea L, et al., Generation and Characterisation of ALX-0171, a Potent Novel Therapeutic Nanobody for the Treatment of Respiratory Syncytial Virus infection. Antimicrobial Agents and Chemotherapy 60(1) 2015
De Vlieger et al., Single-domain Antibodies and their Formatting to Combat Viral Infections, Antibodies 8(1), 1-22, 2019
Duggan S. Caplacizumab: First Global Approval. Drugs (2018) 78:1639-1642
Fleischmann R, et al., A Novel Individualized Treatment Approach in Open-Label Extension Study of Ozoralizumab (ATN-103) in Subjects with Rheumatoid Arthritis on a Background of Methotrexate. Abstract 1311, ACR/ARHP Annual Meeting (2012).
Fridy P C, et al., A Robust Pipeline for Rapid Production of Versatile Nanobody Repertoires. Nat Methods. 2014 December; 11(12): 1253-1260.
Godar M et al. Therapeutic Bispecific Antibody Formats: a Patent Applications Review (1994-2017) Expert Opinion on Therapeutic patents, 2018; 28(3):251-276
Ha, J-H et al. Immunoglobulin Fc Heterodimer Platform Technology: From Design to Applications in Therapeutic Antibodies and Proteins, Front Immunol, 2016; 7:394.
Hamers-Casterman C, et al., Naturally-occurring Antibodies Devoid of Light-chains. Nature 1993, 363:446-448.
Hmila, I. et al., VHH, Bivalent Domains and Chimeric Heavy Chain-only Antibodies with High Neutralizing Efficacy for Scorpion Toxin Aahl′. Mol. Immunol. 2008, 45, 3847-3856.
Hultberg, A et al., Llama-derived Single Domain Antibodies to Build Multivalent, Super Potent and Broadened Neutralizing Anti-viral Molecules. PLoS One 2011, doi: 10.1371/journal.pone.0017665.
Jähnichen S, et al, CXCR4 Nanobodies (VHH-based single variable domains) Potently Inhibit Chemotaxis and HIV-1 Replication and Mobilize Stem Cells. Proc Natl Acad Sci USA. (2010)
Kovalenko O V et al. Atypical Antigen Recognition Mode of a Shark Immunoglobulin New Antigen Receptor (IgNAR) Variable Domain Characterized by Humanization and Structural Analysis, J Biol Chem. 2013, 288:17408-17419
Muyldermans, S and Smider, 2016. Distinct Antibody Species: Structural Differences Creating Therapeutic Opportunities. Current Opinion in Immunology 2016, 40:7-13
Muyldermans, S et al., 1994. Sequence and Structure of VH Domain From Naturally Occurring Camel Heavy Chain Immunoglobulins Lacking Light Chains. Protein Eng. 7: 1129-1135.
Saunders K. O., Conceptual Approach to Modulating Antibody Effector Functions and Circulation Half-Life, Front. Immunol. 10:1296, 2019.
Schoen P, et al., Anti-RANKL Nanobody® ALX-0141 Shows Sustained Biomarker Inhibition in a Phase I Study in Healthy Postmenopausal Women. DOI: 10.1530/boneabs.1.PP135 (2013)
Simmons, D. P et al., Dimerisation Strategies for Shark IgNAR Single Domain Antibody Fragments. J. Immunol. Methods 2006, 315, 171-184.
Svecova D, et al., A Randomized, Double-Blind, Placebo-Controlled Phase 1 Study of Multiple Ascending Doses of Subcutaneous M1095, an Anti-Interleukin-17A/F Nanobody®, in Moderate-to-Severe Psoriasis. J Am Acad Dermatol. 2019 Mar. 26.
Tatusova, T. A, et al. (1999), BLAST 2 Sequences, a New Tool for Comparing Protein and Nucleotide Sequences, FEMS Microbiol Lett. 174:247-250)
Van Roy M, et al., The Preclinical Pharmacology of the High Affinity Anti-IL-6R Nanobody® ALX-0061 Supports its Clinical Development in Rheumatoid Arthritis. Arthritis Research & Therapy (2015) 17:135
Vincke C. et al. General Strategy to Humanize a Camelid Single-Domain Antibody and Identification of a Universal Humanized Nanobody Scaffold, J. Biol Chem. 2009, 284(5):3273-3284
Vu, K. B., et al., 1997. Comparison of Llama VH Sequences from Conventional and Heavy Chain Antibodies. Mol. Immunol. 34: 1121-1131.

SEQUENCE LISTING
(Human IgG1 hinge)
SEQ ID NO: 1
EPKSCDKTHTCPPCP
(Linker-HL1)
SEQ ID NO: 2
EPKIPQPQPKPQPQPQPGGSGSAEAAAKAPKAP
(flexible linker-FL2)
SEQ ID NO: 3
GGGGSGGGGS
(flexible linker-FL 18)
SEQ ID NO: 4
GGGGSGGGGSGGGGS
(flexible linker-FL4)
SEQ ID NO: 5
GGGGSGGGGSGGGGSGGGGSGGGGS
(flexible linker- FL5)
SEQ ID NO: 6
GGGGSGGGGSGGGGSGGGGSGGGGSGGGGS
(flexible linker)
SEQ ID NO: 7
(GGGGS)ₙ
wherein n is an integer selected
from 1 to 10
(rigid linker-RL5)
SEQ ID NO: 8
PAPAPKA
(rigid linker-RL7)
SEQ ID NO: 9
APAPAPAPAPKA
(rigid linker-RL12)
SEQ ID NO: 10
APAPAPAPAP APAPAPAPAPKA
(rigid linker)
SEQ ID NO: 11
(X(PAPAP))ₙKA
wherein n is an integer
selected from 1 to 10, wherein X is
present or absent and is A
(helical linker-RL1)
SEQ ID NO: 12
AEAAAKEAAAKA
(helical linker-RL2)
SEQ ID NO: 13
AEAAAKEAAAKEAAAKA
(helical linker-RL4)
SEQ ID NO: 14
AEAAAKEAAAKEAAAKEAAAKEAAAKA
(helical linker
SEQ ID NO: 15
X(EAAAK)ₙY
wherein n is an integer selected from 1 to 10,
more preferably 2-5 wherein X and
Y are independently present or absent
and is preferably A
Human IgG1 constant region
SEQ ID NO: 16
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREEMTKNQVSLTCLVKGFYPSDIAVEWESNGQPENN
YKTTPPVLDSDGSFFLYSKLTVDKSRWQQGNVFSCSVMHE
ALHNHYTQKSLSLSPGK
Alternative Human IgG1 constant region
SEQ ID NO: 17
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSRDELTKNQVSLTCLVKGFYPSDIAVEWESNGQPENN
YKTTPPVLDSDGSFFLYSKLTVDKSRWQQGNVFSCSVMHE
ALHNHYTQKSLSLSPGK
Mutated Human IgG1 constant region
(Chain A- mutations D399N; K370E;
E356Q)
SEQ ID NO: 18
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Human IgG1 constant region
(Chain B- mutations D399N; K439E;
E357Q)
SEQ ID NO: 19
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Anti-CD3 VHH
SEQ ID NO: 20
EVQLVESGGGLVQPGGSLRLSCAASGDIYKSFDMGWYRQA
PGKQRDLVAVIGSRGNNRGRTNYADSVKGRFTISRDGTGN
TVYLLMNKLRPEDTAIYYCNTAPLVAGRPWGRGTLVTVSS
Anti-PD1 VHH
SEQ ID NO: 21
XVQLVESGGGLVQAGKSLRLSCAASGSIFSIHAMGWFRQA
PGKEREFVAAITWSGGITYYEDSVKGRFTISRDNAKNTVY
LQMNSLKPEDTAIYYCAADRAESSWYDYWGQGTQVTVSS
Wherein X is E or Q
Anti-HEWL VHH
SEQ ID NO: 22
XVQLVESGGGSVQAGGSLRLSCAASGSTDSIEYMTWFRQA
PGKAREGVAALYTHTGNTYYTDSVKGRFTISQDKAKNMAY
LRMDSVKSEDTAIYTCGATRKYVPVRFALDQSSYDYWGQG
TQVTVSS
Wherein X is E or Q
Anti-4HEM VHH
SEQ ID NO: 23
XVQLVESGGGLVQAGGSLRLSCAASESTFSNYAMGWFRQA
PGPEREFVATISQTGSHTYYRNSVKGRFTISRDNAKNTVY
LQMNNMKPEDTAVYYCAAGDNYYYTRTYEYDYWGQGTQVT
VSS
Wherein X is E or Q
Anti-PDL1 VHH
SEQ ID NO: 24
EVQLVESGGGLVQPGGSLRLSCAASGFTLDYYAKCWFRQA
PGKEREWVSCISSSDGSTYYADSVKGRFTISRDNAKNTVY
LQMNSLKPEDTAVYFCAARHGGPLTVEYFFDYWGQGTQVT
VSS
(CH2/CH3 IgG4-3)
SEQ ID NO: 25
APEFLGGPSVFLFPPKPKDTLMISRTPEVTCVVVDVSQED
PEVQFNWYVDGVEVHNAKTKPREEQFNSTYRVVSVLTVLH
QDWLNGKEYKCKVSNKGLPSSIEKTISKAKGQPREPQVYT
LPPSQEEMTKNQVSLTCLVKGFYPSDIAVEWESNGQPENN
YKTTPPVLDSDGSFFLYSKLTVDKSRWQEGNVFSCSVMHE
ALHNHYTQKSLSLSLGK
(Fc Region IgG4-1)
SEQ ID NO: 26
APEFLGGPSVFLFPPKPKDTLMISRTPEVTCVVVDVSQED
PEVQFNWYVDGVEVHNAKTKPREEQFNSTYRVVSVLTVLH
QDWLNGKEYKCKVSNKGLPSSIEKTISKAKGQPREPQVYT
LPPSQEEMTKNQVSLTCLVKGFYPSDIAVEWESNGQPENN
YKTTPPVLDSDGSFFLYSRLTVDKSRWQEGNVFSCSVMHE
ALHNHYTQKSLSLSLGK
natural human CH3
SEQ ID NO: 27
GQPREPQVYTLPPSREEMTKNQVSLTCLVKGFYPSDIAVE
WESNGQPENNYKTTPPVLDSDGSFFLYSKLTVDKSRWQQG
NVFSCSVMHEALHNHYTQKSLSLSPGK
Alternative natural human CH3
SEQ ID NO: 28
GQPREPQVYTLPPSRDELTKNQVSLTCLVKGFYPSDIAVE
WESNGQPENNYKTTPPVLDSDGSFFLYSKLTVDKSRWQQG
NVFSCSVMHEALHNHYTQKSLSLSPGK
natural human CH2
SEQ ID NO: 29
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAK
Mutated CH3 domain
SEQ ID NO: 30
(Chain A-mutations D399N; K370E; E356Q)
GQPREPQVYTLPPSRQEMTKNQVSLTCLVEGFYPSDIAVE
WESNGQPENNYKTTPPVLNSDGSFFLYSKLTVDKSRWQQG
NVFSCSVMHEALHNHYTQKSLSLSPGK
Mutated CH3 domain
(Chain B- mutations D399N; K439E; E357Q)
SEQ ID NO: 31
GQPREPQVYTLPPSREQMTKNQVSLTCLVKGFYPSDIAVE
WESNGQPENNYKTTPPVLNSDGSFFLYSKLTVDKSRWQQG
NVFSCSVMHEALHNHYTQESLSLSPGK
human IgG1 hinge variant
SEQ ID NO: 32
EPKSSDKTHTCPPCP
human IgG1 hinge variant
SEQ ID NO: 33
EPKSSDKTHTSPPSP
human IgG1 hinge variant
SEQ ID NO: 34
DKTHTCPPC
SEQ ID NO: 35
human IgG2 hinge
ERKCCVECPPCP
human IgG2 hinge variant
SEQ ID NO: 36
ERKSSVECPPCP
human IgG2 hinge variant
SEQ ID NO: 37
ERKSSVESPPCP
human IgG2 hinge variant
SEQ ID NO: 38
ERKSSVESPPSP
human IgG3 hinge
SEQ ID NO: 39
ELKTPLGDTTHTCPRCPEPKSCDTPPPCPRCPEPKSCDTP
PPCPRCPEPKSCDTPPPCPRCP
human IgG3 hinge variant
SEQ ID NO: 40
EPKSSDTPPPCPRCP
human IgG3 hinge variant
SEQ ID NO: 41
EPKSSDTPPPSPRCP
human IgG3 hinge variant
SEQ ID NO: 42
EPKSSDTPPPSPRSP
human IgG4 hinge
SEQ ID NO: 43
ESKYGPPCPSCP
human IgG4 hinge variant
SEQ ID NO: 44
ESKYGPPCPPCP
human IgG4 hinge variant
SEQ ID NO: 45
ESKYGPPSPSCP
human IgG4 hinge variant
SEQ ID NO: 46
ESKYGPPSPSSP
human IgG1 Fc region variant
SEQ ID NO: 47
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSRDELTKNQVSLTCLVKGFYPSDIAVEWESNGQPENN
YKTTPPVLDSDGSFFLYSKLTVDKSRWQQGNVFSCSVMHE
ALHNHYTQKSLSLSPG
human IgG2 Fc region
SEQ ID NO: 48
APPVAGPSVFLFPPKPKDTLMISRTPEVTCVVVDVSHEDP
EVQFNWYVDGVEVHNAKTKPREEQFNSTFRVVSVLTVVHQ
DWLNGKEYKCKVSNKGLPAPIEKTISKTKGQPREPQVYTL
PPSREEMTKNQVSLTCLVKGFYPSDISVEWESNGQPENNY
KTTPPMLDSDGSFFLYSKLTVDKSRWQQGNVFSCSVMHEA
LHNHYTQKSLSLSPGK
human IgG2 variant
SEQ ID NO: 49
APPVAGPSVFLFPPKPKDTLMISRTPEVTCVVVDVSHEDP
EVQFNWYVDGVEVHNAKTKPREEQFNSTFRVVSVLTVVHQ
DWLNGKEYKCKVSNKGLPAPIEKTISKTKGQPREPQVYTL
PPSREEMTKNQVSLTCLVKGFYPSDIAVEWESNGQPENNY
KTTPPMLDSDGSFFLYSKLTVDKSRWQQGNVFSCSVMHEA
LHNHYTQKSLSLSPGK
human IgG3 Fc region
SEQ ID NO: 50
APELLGGPSVFLFPPKPKDTLMISRTPEVTCVVVDVSHED
PEVQFKWYVDGVEVHNAKTKPREEQYNSTFRVVSVLTVLH
QDWLNGKEYKCKVSNKALPAPIEKTISKTKGQPREPQVYT
LPPSREEMTKNQVSLTCLVKGFYPSDIAVEWESSGQPENN
YNTTPPMLDSDGSFFLYSKLTVDKSRWQQGNIFSCSVMHE
ALHNRFTQKSLSLSPGK
Signal Peptide
SEQ ID NO: 51
MEWSWVFLFFLSVTTGVHS
Epitope Tag
SEQ ID NO: 52
HHHHHH
Mutated Fc (KB081 Fc)
SEQ ID NO: 53
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSRQEMTKNQVSLTCLVEGFYPSDIAVEWESNGQPENN
YKTTPPVLQSDGSFFLYSKLTVDKSRWQQGNVFSCSVMHE
ALHNHYTQKSLSLSPGK
Mutated Fc (KB082 Fc)
SEQ ID NO: 54
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREQMTKNQVSLTCLVKGFYPSDIAVEWESNGQPENN
YKTTPPVLQSDGSFFLYSKLTVDKSRWQQGNVFSCSVMHE
ALHNHYTQESLSLSPGK
Mutated Fc (KB083 Fc)
SEQ ID NO: 55
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVKT
LPPKRQEMTKNQVSLTCLVEGFYPSDIAVEWESNGQPENN
YKTTPPVLQSDGSFFLYSKLTVDKSRWQQGNVFSCSVMHE
ALHNHYTQKSLSLSPGK
Mutated Fc (KB084 Fc)
SEQ ID NO: 56
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVDT
LPPDREQMTKNQVSLTCLVKGFYPSDIAVEWESNGQPENN
YKTTPPVLQSDGSFFLYSKLTVDKSRWQQGNVFSCSVMHE
ALHNHYTQESLSLSPGK
Mutated Fc (KB085 Fc)
SEQ ID NO: 57
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
YPPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB086 Fc)
SEQ ID NO: 58
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPKREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB087 Fc)
SEQ ID NO: 59
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
HPPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB088 Fc)
SEQ ID NO: 60
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPWREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB089 Fc)
SEQ ID NO: 61
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPMRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB090 Fc)
SEQ ID NO: 62
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
YPPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB091 Fc)
SEQ ID NO: 63
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSKQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB092 Fc)
SEQ ID NO: 64
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVRT
LPPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB093 Fc)
SEQ ID NO: 65
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPKRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (CKB094 Fc)
SEQ ID NO: 66
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYL
LPPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB095 Fc)
SEQ ID NO: 67
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYI
LPPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB096 Fc)
SEQ ID NO: 68
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYI
LPPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB097 Fc)
SEQ ID NO: 69
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYL
LPPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB098 Fc)
SEQ ID NO: 70
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSWEQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB099 Fc)
SEQ ID NO: 71
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYV
LPPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB100 Fc)
SEQ ID NO: 72
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYV
LPPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB101 Fc)
SEQ ID NO: 73
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSRQEMTKNQVSLTCLVEGFYPSDIAVEWESNGQPENN
YKTNPPVLNSDGSFFLYSKLTVDKSRWQQGNVFSCSVMHE
ALHNHYTQKSLSLSPGK
Mutated Fc (KB102 Fc)
SEQ ID NO: 74
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LYPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB103 Fc)
SEQ ID NO: 75
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LVPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB104 Fc)
SEQ ID NO: 76
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LTPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB105 Fc)
SEQ ID NO: 77
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LRPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB106 Fc)
SEQ ID NO: 78
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LLPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB107 Fc)
SEQ ID NO: 79
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LGPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB108 Fc)
SEQ ID NO: 80
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LEPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB109 Fc)
SEQ ID NO: 81
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LCPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB110 Fc)
SEQ ID NO: 82
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
WPPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB111 Fc)
SEQ ID NO: 83
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
TPPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB112 Fc)
SEQ ID NO: 84
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
APPSRQEMTKNQVSLTCLVEGFYPSDIAVEWESNGQPENN
YKTTPPVLNSDGSFFLYSKLTVDKSRWQQGNVFSCSVMHE
ALHNHYTQKSLSLSPGK
Mutated Fc (KB113 Fc)
SEQ ID NO: 85
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREQMTKNQVSLTCLVKGFYPSDIAVEWESNGQPENN
YKTTIPVLNSDGSFFLYSKLTVDKSRWQQGNVFSCSVMHE
ALHNHYTQESLSLSPGK
Mutated Fc (KB114 Fc)
SEQ ID NO: 86
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREQMTKNQVSLTCLVKGFYPSDIAVEWESNGQPENN
YKTTGPVLNSDGSFFLYSKLTVDKSRWQQGNVFSCSVMHE
ALHNHYTQESLSLSPGK
Mutated Fc (KB115 Fc)
SEQ ID NO: 87
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSREQMTKNQVSLTCLVKGFYPSDIAVEWESNGQPENN
YKTTEPVLNSDGSFFLYSKLTVDKSRWQQGNVFSCSVMHE
ALHNHYTQESLSLSPGK
Mutated Fc (KB116 Fc)
SEQ ID NO: 88
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LKPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB117 Fc)
SEQ ID NO: 89
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LDPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB118 Fc)
SEQ ID NO: 90
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
RPPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated Fc (KB119 Fc)
SEQ ID NO: 91
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
DPPSREQMTKNQVSLTCLVKGFYPSDIAVEWESNGQPENN
YKTTPPVLNSDGSFFLYSKLTVDKSRWQQGNVFSCSVMHE
ALHNHYTQESLSLSPGK
Mutated CH3 domain of KB083
Chain A (mutations D399Q; K370E; E356Q,
Y349K, S354K)
SEQ ID NO: 92
GQPREPQVKTLPPKRQEMTKNQVSLTCLVEGFYPSDIAVE
WESNGQPENNYKTTPPVLQSDGSFFLYSKLTVDKSRWQQG
NVFSCSVMHEALHNHYTQKSLSLSPGK
)Mutated CH3 domain of KB084 Chain B
(mutations D399Q; K439E; E357Q,
Y349D, S354D)
SEQ ID NO: 93
GQPREPQVDTLPPDREQMTKNQVSLTCLVKGFYPSDIAVE
WESNGQPENNYKTTPPVLQSDGSFFLYSKLTVDKSRWQQG
NVFSCSVMHEALHNHYTQESLSLSPGK
(Mutated CH3 domain of KB110 Chain A
(mutations D399N; K370E; E356Q;
L351W)
SEQ ID NO: 94
GQPREPQVYTWPPSRQEMTKNQVSLTCLVEGFYPSDIAVE
WESNGQPENNYKTTPPVLNSDGSFFLYSKLTVDKSRWQQG
NVFSCSVMHEALHNHYTQKSLSLSPGK
Mutated CH3 domain of KB118 Chain B
(mutations D399N; K439E; E357Q;
L351R)
SEQ ID NO: 95
GQPREPQVYTRPPSREQMTKNQVSLTCLVKGFYPSDIAVE
WESNGQPENNYKTTPPVLNSDGSFFLYSKLTVDKSRWQQG
NVFSCSVMHEALHNHYTQESLSLSPGK
Mutated CH3 domain of KB089 Chain A
(mutations D399N, K370E, E356Q,
S354M)
SEQ ID NO: 96
GQPREPQVYTLPPMRQEMTKNQVSLTCLVEGFYPSDIAVE
WESNGQPENNYKTTPPVLNSDGSFFLYSKLTVDKSRWQQG
NVFSCSVMHEALHNHYTQKSLSLSPGK
Mutated CH3 domain of KB090 Chain B
(mutations D399N, K439E, E357Q,
L351Y)
SEQ ID NO: 97
GQPREPQVYTYPPSREQMTKNQVSLTCLVKGFYPSDIAVE
WESNGQPENNYKTTPPVLNSDGSFFLYSKLTVDKSRWQQG
NVFSCSVMHEALHNHYTQESLSLSPGK
Mutated CH3 domain of KB095 Chain A
(mutations D399N, K370E, E356Q,
T350I)
SEQ ID NO: 98
GQPREPQVYILPPSRQEMTKNQVSLTCLVEGFYPSDIAVE
WESNGQPENNYKTTPPVLNSDGSFFLYSKLTVDKSRWQQG
NVFSCSVMHEALHNHYTQKSLSLSPGK
(Mutated CH3 domain of KB096 Chain B
(mutations D399N, K439E, E357Q,
T350I)
SEQ ID NO: 99
GQPREPQVYILPPSREQMTKNQVSLTCLVKGFYPSDIAVE
WESNGQPENNYKTTPPVLNSDGSFFLYSKLTVDKSRWQQG
NVFSCSVMHEALHNHYTQESLSLSPGK
(Mutated CH3 domain of KB099 Chain A
(mutations D399N, K370E, E356Q,
T350V)
SEQ ID NO: 100
GQPREPQVYVLPPSRQEMTKNQVSLTCLVEGFYPSDIAVE
WESNGQPENNYKTTPPVLNSDGSFFLYSKLTVDKSRWQQG
NVFSCSVMHEALHNHYTQKSLSLSPGK
Mutated CH3 domain of KB100 Chain B
(mutations D399N, K439E, E357Q,
T350V)
SEQ ID NO: 101
GQPREPQVYVLPPSREQMTKNQVSLTCLVKGFYPSDIAVE
WESNGQPENNYKTTPPVLNSDGSFFLYSKLTVDKSRWQQG
NVFSCSVMHEALHNHYTQESLSLSPGK
Mutated CH3 domain of KB105 Chain A
(mutations D399N, K370E, E356Q,
P352R)
SEQ ID NO: 102
GQPREPQVYTLRPSRQEMTKNQVSLTCLVEGFYPSDIAVE
WESNGQPENNYKTTPPVLNSDGSFFLYSKLTVDKSRWQQG
NVFSCSVMHEALHNHYTQKSLSLSPGK
Mutated CH3 domain of KB108 Chain A
(mutations D399N, K370E, E356Q,
P352E)
SEQ ID NO: 103
GQPREPQVYTLEPSRQEMTKNQVSLTCLVEGFYPSDIAVE
WESNGQPENNYKTTPPVLNSDGSFFLYSKLTVDKSRWQQG
NVFSCSVMHEALHNHYTQKSLSLSPGK

1. A polypeptide comprising in a N- to C-terminal fashion an amino acid sequence of formula Ib:
X-[(Abₐ)-(L_{b})]ₘ-(DD)-[(L_{c})-(Ab_{d})]ₙ-Y
wherein m is 0, 1 or an integer greater than 1;
wherein n is 2 or an integer greater than 2;
wherein Abₐ, Ab_{d}, each independently comprise an antigen binding domain comprising one or more complementarity determining regions (CDRs) of an antibody;
wherein X or Y are independently present or absent and comprises an amino acid sequence;
wherein L_{b}, L_{c}, each independently comprises one or more linkers;
wherein L_{c} does not comprise a cleavable linker; and
wherein DD comprises a dimerization domain.

2. The polypeptide of claim 1, wherein the dimerization domain comprises a CH2-CH3 domain of a natural antibody or CH3 mutations that favor heterodimer formation.

3.-21. (canceled)

22. A polypeptide comprising in a N- to C-terminal fashion an amino acid sequence of formula Ic:
X-[(Abₐ)-(L_{b})]ₘ-(DD)-[(L_{c})-(Ab_{d})]ₙ-Y
wherein m is 0, 1 or an integer greater than 1;
wherein n is 0, 1 or an integer greater than 1, provided that m and n are not 0 simultaneously;
wherein Abₐ, Ab_{d}, each independently comprise an antigen binding domain comprising one or more complementarity determining regions (CDRs) of an antibody;
wherein X or Y are independently present or absent and comprises an amino acid sequence;
wherein L_{b}, L_{c}, each independently comprises one or more linkers;
wherein DD comprises a dimerization domain comprising
a CH3 domain comprising one or more mutations at positions corresponding to D399, D/E356 and/or K370 in accordance with EU numbering;
or
a CH3 domain comprising one or more mutations at positions corresponding to D399, E357 and/or K439 in accordance with EU numbering.

23. The polypeptide of claim 22, wherein the CH3 domain comprises:
a. mutations D399N, E356Q and K370E;
b. mutations D399N, E357Q, K439E;
c. mutations D399Q, D/E356Q, K370E, Y349K and S354K;
d. mutations D399N, D/E356Q, K370E and L351W;
e. mutations D399N, D/E356Q, K370E and S354M;
f. mutations D399N, D/E356Q, K370E and T350I;
g. mutations D399N, D/E356Q, K370E and T350V;
h. mutations D399N, D/E356Q, K370E and P352R;
i. mutations D399N, D/E356Q, K370E and P352E;
j. mutations D399Q, D/E356Q and K370E;
k. mutations D399N, D/E356Q, K370E and L351Y;
l. mutations D399N, D/E356Q, K370E, and L351H;
m. mutations D399N, D/E356Q, K370E, and R355K;
n. mutations D399N, D/E356Q, K370E, and Q355K;
o. mutations D399N, D/E356Q, K370E and S354K;
p. mutations D399N, D/E356Q, K370E and T350L;
q. mutations D399N, D/E356Q, K370E and T394N;
r. mutations D399N, D/E356Q, K370E and P352Y;
s. mutations D399N, D/E356Q, K370E and P352V;
t. mutations D399N, D/E356Q, K370E and P352T;
u. mutations D399N, D/E356Q, K370E and P352L;
v. mutations D399N, D/E356Q, K370E and P352G;
w. mutations D399N, D/E356Q, K370E and P352C;
x. mutations D399N, D/E356Q, K370E and L351T;
y. mutations D399N, D/E356Q, K370E and L351A;
z. mutations D399Q, E357Q, K439E, Y349D and S354D;
aa. mutations D399N, E357Q, K439E and L351R;
bb. mutations D399N, E357Q, K439E and L351Y;
cc. mutations D399N, E357Q, K439E and T350I;
dd. mutations D399N, E357Q, K439E and T350V;
ee. mutations D399Q, K439E, E357Q;
ff. mutations D399N, K439E, E357Q, S354K;
gg. mutations D399N, K439E, E357Q, S354W;
hh. mutations D399N, K439E, E357Q, Y349R;
ii. mutations D399N, K439E, E357Q, T350L;
jj. mutations D399N, K439E, E357Q, R355W;
kk. mutations D399N, K439E, E357Q, Q355W;
ll. mutations D399N, K439E, E357Q, P395I;
mm. mutations D399N, K439E, E357Q, P395G;
nn. mutations D399N, K439E, E357Q, P395E;
oo. mutations D399N, K439E, E357Q, P352K;
pp. mutations D399N, K439E, E357Q, P352D; or
qq. mutations D399N, K439E, E357Q, L351D.

24.-56. (canceled)

57. The polypeptide of claim 1, wherein the dimerization domain comprises an amino acid sequence at least 80%, 85%, 90%, 95%, 96%, 97%, 98%, or 99% identical to SEQ ID NO:27 and optionally further comprises an amino acid sequence at least 80%, 85%, 90%, 95%, 96%, 97%, 98%, or 99% identical to SEQ ID NO: 29.

58.-60. (canceled)

61. The polypeptide of claim 1, wherein the polypeptide is selected from the group consisting of the following:
X-(Abₐ₁)-(L_{b1})-(DD)-(L_{c1})-(Ab_{d1})-Y
(formu1a II);
X-(Abₐ₁)-(L_{b1})-(DD)-(L_{c1})-(Ab_{d1})-
(L_{c2})-(Ab_{d2})-Y
(formu1a III);
X-(Abₐ₁)-(L_{b2})-(Abₐ₂)-(L_{b1})-(DD)-
(L_{c1})-(Ab_{d1})-Y
(formu1a IV);
X-(Abₐ₁)-(L_{b2})-(Abₐ₂)-(L_{b1})-(DD)-
(L_{c1})-(Ab_{d1})-(L_{c2})-(Ab_{d2})-Y
(formu1a V);
X-(Abₐ₁)-(L_{b2})-(Abₐ₂)-(L_{b1})-(DD)-
(L_{c1})-(Ab_{d1})-(L_{c2})-(Ab_{d2})-(L_{c3})-
(Ab_{d3})-Y
(formu1a VI);
X-(Abₐ₁)-(L_{b3})-(Abₐ₂)-(L_{b2})-(Abₐ₃)-
(L_{b1})-(DD)-(L_{c1})-(Ab_{d1})-(L_{c2})-(Ab_{d2})-Y
(formu1a VII);
X-(Abₐ₁)-(L_{b3})-(Abₐ₂)-(L_{b2})-(Abₐ₃)-
(L_{b1})-(DD)-(L_{c1})-(Ab_{d1})-(L_{c2})-(Ab_{d2})-
(L_{c3})-(Ab_{d3})-Y
(formu1a VIII).

62. (canceled)

63. The polypeptide of claim 1, wherein the antigen binding domain is selected from the group consisting of a single domain antibody (sdAb), a heavy chain variable region (VH or VHH), a light chain variable region (VL or VLL) a single chain variable fragment (ScFv), a V_{NAR} fragment, and combinations thereof.

64.-79. (canceled)

80. The polypeptide of claim 1, wherein the polypeptide comprises an antigen binding domain that binds to a tumor antigen, an antigen binding domain that binds to an immunomodulator and/or an antigen binding domain that binds to and recruits an immune cell.

81.-90. (canceled)

91. A pharmaceutical composition comprising the polypeptide of claim 1 and a pharmaceutically acceptable carrier.

92. A nucleic acid encoding the polypeptide of claim 1.

93. A vector comprising the nucleic acid of claim 92.

94. A cell expressing the polypeptide of claim 1.

95. (canceled)

96. A kit comprising the polypeptide of claim 1.

97. A kit comprising the nucleic acid of claim 92.

98. A protein complex comprising a first polypeptide and a second polypeptide wherein the first and second polypeptide are identical or different and wherein the polypeptide comprises in a N- to C-terminal fashion an amino acid sequence of formula Ib:
X-[(Abₐ)-(L_{b})]ₘ-(DD)-[(L_{c})-(Ab_{d})]ₙ-Y
wherein m is 0, 1 or an integer greater than 1;
wherein n is 2 or an integer greater than 2;
wherein Abₐ, Ab_{d}, each independently comprise an antigen binding domain comprising one or more complementarity determining regions (CDRs) of an antibody;
wherein X or Y are independently present or absent and comprises an amino acid sequence;
wherein L_{b}, L_{c}, each independently comprises one or more linkers;
wherein L_{c} does not comprise a cleavable linker; and
wherein DD comprises a dimerization domain.

99. A protein complex comprising
a first polypeptide comprising one or more antigen binding domains and a first dimerization domain (DD₁) comprising a CH3 domain comprising one or more mutations at positions corresponding to D399, D/E356 and/or K370 in accordance with EU numbering;
and
a second polypeptide comprising one or more antigen binding domains and a second dimerization domain (DD₂) comprising a CH3 domain comprising one or more mutations at positions corresponding to D399, E357 and/or K439 in accordance with EU numbering wherein the first and second polypeptides form a dimer.

100.-109. (canceled)

110. The protein complex of claim 98, wherein each of the one or more linkers is independently a flexible linker, a helical linker, a rigid linker or a hinge region of an antibody or antigen binding fragment thereof.

111. (canceled)

112. The protein complex of claim 98, wherein the first and second polypeptide each is independently a polypeptide having a formula selected from:
X-(Abₐ₁)-(L_{b1})-(DD)-(L_{c1})-(Ab_{d1})-Y
(formu1a II);
X-(Abₐ₁)-(L_{b1})-(DD)-(L_{c1})-(Ab_{d1})-
(L_{c2})-(Ab_{d2})-Y
(formu1a III);
X-(Abₐ₁)-(L_{b2})-(Abₐ₂)-(L_{b1})-(DD)-
(L_{c1})-(Ab_{d1})-Y
(formu1a IV);
X-(Abₐ₁)-(L_{b2})-(Abₐ₂)-(L_{b1})-(DD)-
(L_{c1})-(Ab_{d1})-(L_{c2})-(Ab_{d2})-Y
(formu1a V);
X-(Abₐ₁)-(L_{b2})-(Abₐ₂)-(L_{b1})-(DD)-
(L_{c1})-(Ab_{d1})-(L_{c2})-(Ab_{d2})-(L_{c3})-
(Ab_{d3})-Y
(formu1a VI);
X-(Abₐ₁)-(L_{b3})-(Abₐ₂)-(L_{b2})-(Abₐ₃)-
(L_{b1})-(DD)-(L_{c1})-(Ab_{d1})-(L_{c2})-(Ab_{d2})-Y
(formu1a VII);
X-(Abₐ₁)-(L_{b3})-(Abₐ₂)-(L_{b2})-(Abₐ₃)-
(L_{b1})-(DD)-(L_{c1})-(Ab_{d1})-(L_{c2})-(Ab_{d2})-
(L_{c3})-(Ab_{d3})-Y
(formu1a VIII).

113. (canceled)

114. The protein complex of claim 98, wherein the protein complex is multispecific.

115.-120. (canceled)

121. A composition comprising the protein complex of claim 98.

122. (canceled)

123. The composition of claim 121, wherein greater than 70%, 75%, 80%, 85%, 90%, 95%, 96%, 97%, 98%, or 99% of the first and second polypeptides exist as dimers.

124.-125. (canceled)

126. A method of treating a disorder or disease comprising administering the protein complex of claim 98.

127. The method of claim 126, wherein the disorder or disease is cancer, an infection or an immune disregulation.

128.-153. (canceled)

154. The method of claim 126, wherein the polypeptide comprises an antigen binding domain that binds to a tumor antigen, an antigen binding domain that binds to an immunomodulator and/or an antigen binding domain that binds to and recruits an immune cell.

155. The protein complex of claim 98, wherein the dimerization domain comprises a CH2-CH3 domain of a natural antibody or CH3 mutations that favor heterodimer formation

156. The protein complex of claim 98, wherein the antigen binding domain comprises a single domain antibody (sdAb), a heavy chain variable region (VH or VHH), a light chain variable region (VL or VLL), a single chain variable fragment (ScFv), or a V_{NAR} fragment.

157. The protein complex of claim 98, wherein the polypeptide comprises an antigen binding domain that binds to a tumor antigen, an antigen binding domain that binds to an immunomodulator and/or an antigen binding domain that binds to and recruits an immune cell.

158. The protein complex of claim 98, wherein the first and/or second polypeptide is conjugated to a therapeutic moiety, detectable moiety or to a protein allowing an extended half-life or is attached to nanoparticle.

159. A composition comprising the protein complex of claim 157.

160. A method of treating a disorder or disease comprising administering the protein complex of claim 157.

161. A protein complex comprising a first polypeptide and a second polypeptide wherein the first and second polypeptide are identical or different and comprise in a N- to C-terminal fashion an amino acid sequence of formula Ic:
X-[(Abₐ)-(L_{b})]ₘ-(DD)-[(L_{c})-(Ab_{d})]ₙ-Y
wherein m is 0, 1 or an integer greater than 1;
wherein n is 0, 1 or an integer greater than 1, provided that m and n are not 0 simultaneously;
wherein Abₐ, Ab_{d}, each independently comprise an antigen binding domain comprising one or more complementarity determining regions (CDRs) of an antibody;
wherein X or Y are independently present or absent and comprises an amino acid sequence;
wherein L_{b}, L_{c}, each independently comprises one or more linkers;
wherein DD is the first dimerization domain (DD₁) in the first polypeptide and the second dimerization domain (DD₂) in the second polypeptide and wherein DD₁ comprises a CH3 domain comprising one or more mutations at positions corresponding to D399, D/E356 and/or K370 in accordance with EU numbering and wherein DD₂ comprises a CH3 domain comprising one or more mutations at positions corresponding to D399, E357 and/or K439 in accordance with EU numbering.
